(19) United States
(12) Patent Application Publication
(10) Pub. No.: US 2006/0263864 A1
(43) Pub. Date: Nov. 23, 2006

(54) Methods for improving secondary metabolite production of fungi

(76) Inventors: Robert Busby, Weymouth, MA (US); Brian Cali, Arlington, MA (US); Peter Hecht, Newton, MA (US); Douglas Holtzman, Jamaica Plain, MA (US); Kevin T. Madden, Arlington, MA (US); Mary Maxon, San Francisco, CA (US); G. Todd Milne, Brookline, MA (US); Thea Norman, Belmont, MA (US); John C. Royer, Lexington, MA (US); Jeff Silva, Beverly, MA (US); Eric F. Summers, Brookline, MA (US); Lixin Zhang, Lexington, MA (US); Maria Mayorga, Somerville, MA (US); Toby Feibelman, Lincoln, MA (US); Reed Doten, Framingham, MA (US)

Correspondence Address: FISH & RICHARDSON PC, P.O. BOX 1022, MINNEAPOLIS, MN, 55440-1022, US

(21) Appl. No.: 10/309,407

(22) Filed: Dec. 3, 2002

Related U.S. Application Data
(63) Continuation of application No. 09/691,499, filed on Oct. 18, 2000, now abandoned.
Application No. 09/691,499 is a continuation-in-part of application No. 09/487,558, filed on Jan. 19, 2000, now Pat. No. 6,949,356.
(60) Provisional application No. 60/160,587, filed on Oct. 20, 1999.

(51) Int. Cl. C12P 17/06; C12P 7/62; C12N 15/74; C12N 1/16

(52) U.S. Cl. 435/125; 435/135; 435/484; 435/254.3

(57) ABSTRACT

Methods for improving secondary metabolite production by modulating certain genes involved in secondary metabolite production, including genes encoding CreA, AreA, GanB, Gna3, FadA, Gna1, Gpa1, RfeH, An09, PacC, Lys14, LovU, Ste7, Pde2, Nc1, Vps34 and fungal homologs thereof.

### RELATED APPLICATION INFORMATION

This application is a continuation-in-part of U.S. application Ser. No. 09/691,499, filed Oct. 18, 2000, which is a continuation-in-part of U.S. application Ser. No. 09/487,558, filed Jan. 19, 2000, which claims priority from U.S. provisional application Ser. No. 60/160,587, filed Oct. 20, 1999, all of which are hereby incorporated by reference.

### TECHNICAL FIELD

The invention relates to the production of secondary metabolites by fungi. More particularly, the invention relates to improvement of production of commercially important secondary metabolites by fungi.

### BACKGROUND

Secondary metabolite production by various fungi has been an extremely important source of a variety of therapeutically significant pharmaceuticals. β-lactam antibacterials such as penicillin and cephalosporin are produced by Penicillium chrysogenum and Acremonium chrysogenum, respectively, and these compounds are by far the most frequently used antibacterials (reviewed in Luengo and Penalva, Prog. Ind. Microbiol. 29: 603-38 (1994); Jensen and Demain, Biotechnology 28: 239-68 (1995); Brakhage, Microbiol. Mol. Biol. Rev. 62: 547-85 (1998)). Cyclosporin A, a member of a class of cyclic undecapeptides, is produced by Tolypocladium inflatum. Cyclosporin A dramatically reduces morbidity and increases survival rates in transplant patients (Borel, Prog. Allergy 38: 9-18 (1986)). In addition, several fungal secondary metabolites are cholesterol lowering drugs, including lovastatin that is made by Aspergillus terreus and several other fungi (Alberts et al., Proc. Natl. Acad. Sci. USA 77: 3957-3961 (1980)). These and many other fungal secondary metabolites have contributed greatly to health care throughout the world (see Demain, Ciba Found Symp 171: 3-16 (1992); Bentley, Crit. Rev. Biotechnol. 19: 1-40 (1999)).

Unfortunately, many challenges are encountered between the detection of a secondary metabolite activity to production of significant quantities of pure drug. Thus, efforts have been made to improve the production of secondary metabolites by fungi. Some of these efforts have attempted to improve production by modification of the growth medium or the bioreactor used to carry out the fermentation. Buckland et al., in Topics in Industrial Microbiology: Novel Microbial products for Medicine and Agriculture, pp. 161-169, Elsevier, Amsterdam (1989) discloses improved lovastatin production by modification of carbon source and also teaches the superiority of a hydrofoil axial flow impeller in the bioreactor. Other efforts have involved strain improvements, either through re-isolation or random mutagenesis. Agathos et al., J. Ind. Microbiol. 1: 39-48 (1986), teaches that strain improvement and process development together resulted in a ten-fold increase in cyclosporin A production. While important, studies of these types have still left much room for improvement in the production of secondary metabolites.

More recently, strains have been improved by manipulation of the genes encoding the biosynthetic enzymes that catalyze the reactions required for production of secondary metabolites. Penalva et al., Trends Biotechnol. 16: 483-489 (1998) discloses that production strains of P. chrysogenum have increased copy number of the penicillin synthesis structural genes. Other studies have modulated expression of other biosynthetic enzyme-encoding genes, thereby affecting overall metabolism in the fungus. Mingo et al., J. Biol. Chem. 21: 14545-14550 (1999), demonstrate that disruption of phacA, a gene required for phenylacetate catabolism in A. nidulans, leads to increased penicillin production, probably by allowing increased availability of phenylacetate for secondary metabolism. Similarly, disruption of the gene encoding aminoadipate reductase in P. chrysogenum increased penicillin production, presumably by eliminating competition for the substrate alpha-aminoadipate (Casquiero et al., J. Bacteriol. 181: 1181-1188 (1999)).

Thus, genetic manipulation holds promise for improving production of secondary metabolites. Genetic manipulation to increase the activity of biosynthetic enzymes for secondary metabolite production or to decrease the activity of competing biosynthetic pathways has proven effective for improving production. Maximum benefit might be achieved by combining several strategies of manipulation. For example, modulating the expression of genes that regulate the biosynthetic enzyme-encoding genes might improve production. In addition, genetic manipulation could be used to impact upon the challenges that are encountered in the fermentor run or downstream processing (e.g. energy cost, specific production of desired metabolite, maximal recovery of metabolite, cost of processing waste from fermentations). There is, therefore, a need for methods for improving secondary metabolite production in a fungus, comprising modulating the expression of a gene involved in regulation of secondary metabolite production. Ideally, such methods should be able to provide a means to modulate parameters important in production of secondary metabolites, including, yield, productivity, efflux/excretion, production of side products or non-desired secondary metabolites, strain characteristics such as morphology, conditional lysis, or resistance to the deleterious effects of exposure to a secondary metabolite.

### SUMMARY

The invention provides methods for improving secondary metabolite production in a fungus, comprising modulating the expression of a gene involved in regulation of secondary metabolite production. The methods according to the invention provide increased yield, increased productivity, increased efflux/excretion, decreased production of side products or non-desired secondary metabolites, altered strain characteristics and/or conditional lysis, or increased resistance to the deleterious effects of exposure to a secondary metabolite.

The several aspects of the methods according to the invention are preferably achieved by overexpression of regulatory genes, expression of dominant mutant variants of regulatory genes, use of peptide activators or inhibitors of regulatory gene function, use of small molecule activators or inhibitors of regulatory gene function, and conditional expression of regulatory genes. These factors preferably are or modulate transcription factors, transmembrane transporters, proteins that mediate secretion, kinases, G-proteins, cell surface receptors, GTPase activating proteins, guanine nucleotide exchange factors, phosphatases, proteases, phosphodiesterases, bacterial protein toxins, importins, RNA-binding proteins, SCF complex components, adherins, or biosynthetic pathways.

The methods of the invention can be used to improve production of any secondary metabolite, including, lovastatin, penicillin, geodin, norsolorinic acid, N-Acetylvalyl-N-[2-(1H-indol-3-yl)ethenyl]-N^{É}-methylphenylalaninamide (CAS 124727-69-1; a modified tripeptide), methyl 3,4,5-trimethoxy-2-[[2-[(3pyridinylcarbonylamino]benzoyl]amino]benzoate (CAS 81469-77-1; an alkaloid), and osoic acid 3-methyl ether, 1-methyl ester (CAS 577-64-0; a polyketide).

The invention further provides for achieving the aspects described in the invention by combinatorial manipulation. Combinatorial manipulation is the simultaneous use of multiple methods and/or multiple factors to achieve the aspects of the invention. Methods for achieving the aspects of the invention are preferably by the overexpression of regulatory genes, expression of dominant mutant variants of regulatory genes, use of peptide activators or inhibitors, use of small molecule activators or inhibitors, and conditional expression of regulatory genes. The preferred factors are as described above.

The invention further provides genetically modified fungi, wherein the genetically modified fungi have an ability to produce secondary metabolites and the ability of the genetically modified fungus to produce secondary metabolites has been improved by any of the methods according to the invention.

The invention also provides a method for making a secondary metabolite, the method comprising culturing a genetically modified fungus according to the invention under conditions suitable for the production of secondary metabolites.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 shows the ability of PUMP1 (AAD34558) from Aspergillus terreus to confer lovastatin resistance to a yeast strain.

FIG. 2 shows the impact of yeast genetics and genomics on fungal genetics. Arrows indicate which genes or gene products act on other genes or gene products.

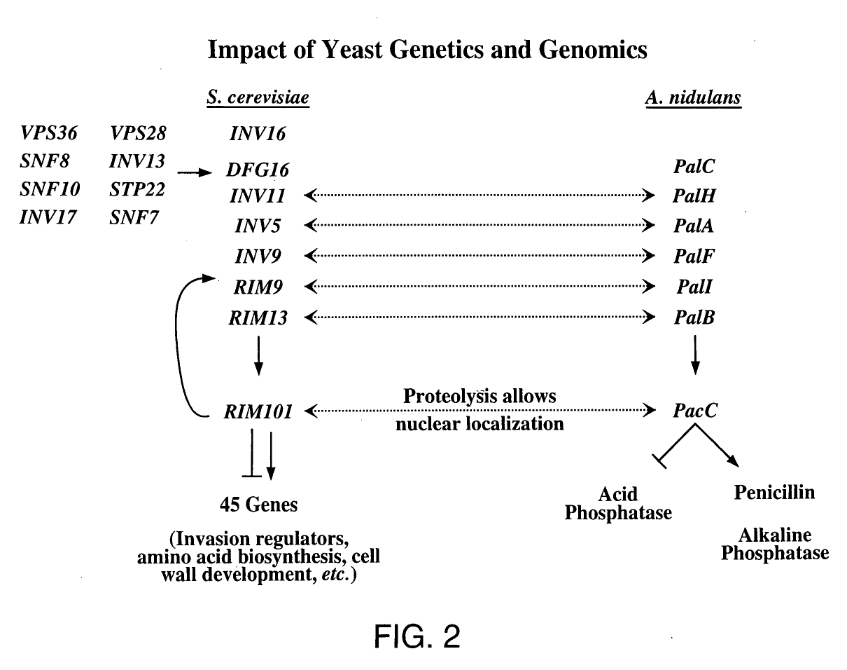

FIG. 3 shows representative box plots presentations of lovastatin production data from shake flask experiments. Data from strains that express a particular regulator (e.g., pacCVP16) are displayed with appropriate negative (EMPTY or GUS) and positive (lovE) controls from the same shake flask experiment.

FIG. 4 is a pair of graphs depicting the effect of CreA expression on lovastatin production in A. terreus.

FIG. 5 is a pair of graphs depicting the effect of AreA expression on lovastatin production in A. terreus.

FIG. 6 is a pair of graphs depicting the effect of pacC on lovastatin production in A. terreus.

FIG. 7 is a set of graphs depicting the effect of ganB, ganB^{G45R}, gna3, and gna3^{G44R} expression on lovastatin production in A. terreus.

FIG. 8 is a set of graphs depicting the effect of fadA^{G42R}, gpa1^{G42R}, gpa1^{Q204L}, and gna1^{G42R} expression on lovastatin production in A. terreus.

FIG. 9 is a set of graphs depicting the effect of inducible expression of gna3^{G44R} on lovastatin production. in A. terreus

FIG. 10 is a set of graphs depicting the effect of rfeH (Pc23) expression on lovastatin production in A. terreus.

FIG. 11 is a set of graphs depicting the effect of VP16-rfeC (An09) and rfeC expression on lovastatin production in A. terreus.

FIG. 12 is a set of graphs depicting the effect various regulators on norsolorinic acid production in A. terreus.

FIG. 13 is a set of graphs depicting the effect of various regulators on lovastatin production in A. terreus.

FIG. 14 is set of graphs depicting the effect of various regulators on penicillin production in P. chrysogenum.

FIG. 15 is set of graphs depicting the effect of Ustilago maydis ste7 expression on lovastatin production in A. terreus.

FIG. 16 is set of graphs depicting the effect of S. cerevisiae VPS34 expression on lovastatin production in A. terreus.

FIG. 17 is set of graphs depicting the effect of N. crassa nc1 expression on lovastatin production in A. terreus.

FIG. 18 is set of graphs depicting the effect of S. cerevisiae PDE2 expression on lovastatin production in A. terreus.

### DETAILED DESCRIPTION

The invention relates to the production of secondary metabolites by fungi. More particularly, the invention relates to improvement of production of commercially important secondary metabolites by fungi. The references cited herein evidence the level of knowledge in the field and are therefore incorporated by reference in their entirety. In the event of a conflict between a cited reference and the present specification, the latter shall prevail.

The invention provides methods for improving secondary metabolite production in a fungus, comprising modulating the expression of a gene involved in regulation of secondary metabolite production. In certain embodiments, the methods comprise modulating the expression of more than one gene involved in regulation of secondary metabolite production.

Methods for Improving Secondary Metabolite Production by Improving Yield of the Metabolite

In a first aspect, the invention provides methods for improving production of a secondary metabolite by a fungus by increasing the yield of the secondary metabolite produced by the fungus. The methods according to this aspect of the invention comprise modulating the expression of a gene involved in regulation of secondary metabolite production in a manner that improves the yield of the secondary metabolite.

Preferably, for this aspect of the invention, when the secondary metabolite is isopenicillin N, then the modulation is not mediated by the transcription factor CPCR1; when the secondary metabolite is sterigmatocystin, then the modulation is not through AflR, FadA, or FluG; when the secondary metabolite is aflatoxin, then the modulation is not through AflR; when the secondary metabolite is penicillin and the fungus is Aspergillus nidulans, then the modulation is not through mutations that result in expression of truncated forms of PacC or constitutively active forms of FadA; when the gene involved in regulation of secondary metabolite production is from Saccharomyces cerevisiae, then the modulation is not through decreased activity or expression of Bem2, Hog1, Ira1, Rim15, Sfl1, Srb11, Ssd1, Swi4, Tpk3 or though increased activity or expression of Afl1, Cdc25, Dhh1, Hap4, Inv11, Inv13, Inv5, Inv7, Inv9, Mcm1, Mep2, Mga1, Msn1, Msn5, Mss11, Pet9, Pho23, Ptc1, Rim101, Rim13, Rim9, Snf8, Stp22, Tpk2, Vps28, Vps36, or Ypr1; and when the secondary metabolite is lovastatin and the fungus is A. terreus, the modulation is not mediated by lovE.

As used herein, the phrase “modulate production of a secondary metabolite” refers to a positive or negative or desirable change in one or more of the variables or values that affect the process or results of production of the secondary metabolite in a liquid or solid state fungal fermentation. These positive or negative or desirable changes include, without limitation, an increase or decrease in the amount of a secondary metabolite being produced (in absolute terms or in quantity per unit volume of fermentation broth or per unit mass of solid substrate); a decrease in the volume of the broth or the mass/quantity of substrate required for the production of sufficient quantities; a decrease in the cost of raw materials and energy, the time of fermentor or culture run, or the amount of waste that must be processed after a fermentor run; an increase or decrease in the specific production of the desired metabolite (both in total amounts and as a fraction of all metabolites and side products made by the fungus); an increase or decrease in the percent of the produced secondary metabolite that can be recovered from the fermentation broth or culture; and an increase in the resistance of an organism producing a secondary metabolite to possible deleterious effects of contact with the secondary metabolite.

A “secondary metabolite” is a compound, derived from primary metabolites, that is produced by an organism, is not a primary metabolite, is not ethanol or a fusel alcohol, and is not required for growth under standard conditions. Secondary metabolites are derived from intermediates of many pathways of primary metabolism. These pathways include, without limitation, pathways for biosynthesis of amino acids, the shikimic acid pathway for biosynthesis of aromatic amino acids, the polyketide biosynthetic pathway from acetyl coenzyme A (CoA), the mevalonic acid pathway from acetyl CoA, and pathways for biosynthesis of polysaccharides and peptidopolysaccharides. Collectively, secondary metabolism involves all primary pathways of carbon metabolism (Fungal Physiology, Chapter 9 pp 246-274 ed DH Griffin (1994)). “Secondary metabolites” also include intermediate compounds in the biosynthetic pathway for a secondary metabolite that are dedicated to the pathway for synthesis of the secondary metabolite. “Dedicated to the pathway for synthesis of the secondary metabolite” means that once the intermediate is synthesized by the cell, the cell will not convert the intermediate to a primary metabolite. “Intermediate compounds” also include secondary metabolite intermediate compounds which can be converted to useful compounds by subsequent chemical conversion or subsequent biotransformation. As such, providing improved availability of such intermediate compounds would still lead to improved production of the ultimate useful compound, which itself may be referred to herein as a secondary metabolite. The yeast Saccharomyces cerevisiae is not known to produce secondary metabolites. The term “primary metabolite” means a natural product that has an obvious role in the functioning of almost all organisms. Primary metabolites include, without limitation, compounds involved in the biosynthesis of lipids, carbohydrates, proteins, and nucleic acids. The term “increasing the yield of the secondary metabolite” means increasing the quantity of the secondary metabolite present in the total fermentation broth per unit volume of fermentation broth.

A “gene involved in regulation of secondary metabolite production” is a gene, other than a gene encoding a biosynthetic enzyme for the secondary metabolite to be produced, which modulates secondary metabolite production involving yield, productivity, efflux/excretion, production of side products or non-desired secondary metabolites, strain characteristics and/or conditional lysis, or resistance to the deleterious effects of exposure to a secondary metabolite. A “biosynthetic enzyme for the secondary metabolite to be produced” is a molecule that catalyzes the conversion of a substrate to a product, including an intermediate product, in the biosynthetic pathway for the secondary metabolite for which production is being improved. An alternative term, “biosynthetic enzyme”, as used herein refers to a molecule that catalyzes the conversion of a substrate to a product, including an intermediate product, in a biosynthetic pathway other than the biosynthetic pathway for the secondary metabolite for which production is being improved.

As used for all aspects of the invention, the term “modulating the expression of a gene” means affecting the function of a gene's product, preferably by increasing or decreasing protein activity through mutation, creating a new protein activity through mutation; increasing or decreasing transcription, increasing or decreasing translation, increasing or decreasing post-translational modification, altering intracellular localization, increasing or decreasing translocation from one cellular location to another, increasing or decreasing protein activity by interaction of the protein with another molecule, or creating a new protein activity by interaction of the protein with another molecule. In some cases, such modulation is achieved simply by allowing or causing the expression of an exogenously supplied nucleic acid or gene. In some cases other exogenously supplied molecules may mediate the modulation. The modulation is not achieved, however, by simply randomly mutagenizing the fungus, either spontaneously or by chemical means.

As used for all aspects of the invention, “mutation” means an alteration in DNA sequence, either by site-directed or random mutagenesis. Mutation encompasses point mutations as well as insertions, deletions, or rearrangements. A “mutant” is an organism containing one or more mutations.

In certain embodiments of the methods according to this aspect of the invention, the modulation is overexpression of the gene. “Overexpression of the gene” means transcription and/or translation and/or gene product maturation at a rate that exceeds by at least two-fold, preferably at least five-fold, and more preferably at least ten-fold, the level of such expression that would be present under similar growth conditions in the absence of the modulation of expression of the gene. In instances where heterologous genes are being expressed, any level of expression is, by definition, considered overexpression.

“Similar growth conditions” means similar sources of nutrients such as carbon, nitrogen, and phosphate, as well as similar pH, partial oxygen pressure, temperature, concentration of drugs or other small molecules, and a similar substrate for growth, whether solid, semi-solid, or liquid.

Preferred genes according to this aspect of the invention include, without limitation, AAD34561, AAD34562, abaA, ACE2, ADR1, AFL1, aflR, AFT1, amyR, areA, ASH1, BAP2, BCY1, CAT8, CDC24, CDC25, CDC28, CDC42, CDC55, CLB2, creA, CTS1, CUP9, CYR1, DFG16, DHH1, DPH3, ELM1, facB, FLO1, FLO11, FLO8, FUS3, GCN2, GCN4, GCR1, GCR2, GLN3, GPA1, GPA2, GPR1, GRR1, GTS1, HAP1, HAP4, HIP1, HMS1, HMS2, HOG1, HSL1, HXK2, IME1, IME4, INO2, INV11, INV13, INV16, INV5, INV7, INV9, KSS1, LEU3, lovE, LYS14, MAC1, MCM1, MEP1, MEP2, MET28, MET31, MET4, metR, MGA1, MIG1, MIG2, MSN1, MSN2, MSN4, MSN5, MSS11, MTH1, NPR1, nreB, NRG1, OAF1, pacC, PBS2, PDE2, PET9, PHD1, PHO2, PHO4, PHO85, pkaR, PPR1, PTC1, PUT3, RAS1, RAS2, RGS2, RIM101, RIM13, RIM15, RIM9, ROX1, RRE1, SCH9, sconB, SFL1, SHO1, SHR3, SIN3, SIP4, SKN7, SNF1, SNF2, SNF7, SNF8, SOK2, SRB10, SRB11, SRB8, SRB9, sreA, sreP, SRV2, SSD1, SSN6, SST2, STE11, STE11, STE20, STE50, STE7, STP22, SWI4, SWI6, tamA, TEC1, TPK1, TPK2, TPK3, TUP1, UaY, UGA3, URE2, VPS28, VPS36, WHI3, YMR077c, YNL255c, YPR1, ZAP1, GanB, Gna3, FadA, Gna1, RfeH (PC23), RfeC (An09), lovU, Ste7, Nc1, Vps34, genes encoding bacterial protein toxins, and any fungal homologs of the aforementioned genes. Tables 5 and 6 include nucleic acid sequences for these genes as well as the predicted amino acid sequences of the proteins they encode. Homologs of these genes and proteins from other fungal species are also useful.

In certain embodiments of the methods according to this aspect of the invention, the modulation is expression of a dominant mutation of the gene. A “dominant mutation” is an allele of a gene that encodes a protein capable of changing the phenotype of an organism more than a non-mutated form of the gene. Dominant mutations include, without limitation, mutations that encode a protein capable of changing the phenotype of an organism even when a non-mutant form of this gene (or its homologs) is resident in the organism. Preferred dominant mutations include dominant negative mutations, dominant positive mutations, and dominant neomorphic mutations. A “dominant negative mutation” is a dominant mutation that achieves its phenotypic effect by interfering with some function of the gene or gene product from which it was derived, or from a homolog thereof. A “dominant positive mutation” is a dominant mutation that achieves its phenotypic effect by activating some function of the gene or gene product from which it was derived, or from a homolog thereof. A “dominant neomorphic mutation” is a dominant mutation that achieves the phenotypic effect of providing a novel function to the gene or gene product from which it was derived, or from a homolog thereof. Preferred dominant mutations according to this aspect of the invention include:

1. Mutations that result in increased or decreased stability of the transcript of a gene.

2. Mutations that result in increased or decreased stability of the product of translation:

For example, specific sequences near the amino terminus of a protein have been shown to cause increased or decreased protein stability. Similarly, sequences elsewhere in the protein, such as those required for ubiquitin-dependent degradation, can be mutated to increase the stability of a protein.

3. Amino acid substitutions that mimic post-translational modifications: For example, phosphorylation has been demonstrated to positively or negatively regulate the activity of a variety of proteins, including transcription factors and kinases. Phosphorylation most commonly occurs on serine, threonine, and tyrosine residues; in some instances residues such as aspartate and histidine can be phosphorylated. Mutations that mimic constitutive dephosphorylation can be produced by mutating the coding sequence of the phosphorylated residue to the coding sequence of an amino acid that cannot be phosphorylated and does not have a negatively charged side chain (e.g. alanine). Alternatively, substitutions that result in changing serine, threonine, or tyrosine residues to charged amino acids such as glutamate or aspartate can result in an allele that mimics constitutive phosphorylation.

Proteolytic cleavage is another post-translational mechanism for regulating the activity of a protein. Mutations that result in truncation of a protein might mimic activation by proteolysis. Mutations that change amino acids required for proteolysis could activate proteins that are negatively regulated by proteolysis.

4. Amino acid substitutions that promote or inhibit the binding of small molecules such as ATP, cAMP, GTP or GDP: For example, ATP is a co-factor for many enzymatic reactions, and the nucleotide-binding domains of these proteins are highly conserved. Lysine to arginine substitutions in the nucleotide-binding domain frequently result in inhibition of enzymatic activity. Enzymatically inactive proteins could be dominant negative molecules, acting by sequestering substrates from functional enzymes.

cAMP is required for the activation of protein kinase A. Protein kinase A consists of regulatory subunits and catalytic subunits. The binding of cAMP to the negative regulatory subunit relieves its inhibition of the catalytic subunit. Therefore, mutations that prevent cAMP binding could result in constitutive inactivation of protein kinase A.

G-proteins are a class of proteins that bind the nucleotides GTP and GDP. The GTP-bound form of these proteins is active, and hydrolysis of GTP to GDP results in the inactivation of the protein. Conserved substitutions can be made to lock G-proteins in either the GTP- or GDP-bound form, thus causing constitutive activation or inactivation.

5. Mutations in portions of genes that encode regulatory domains of proteins: For example, many proteins, including kinases, contain regulatory domains that function as mechanisms to control the timing of activation. Mutations in these domains might result in constitutive activation of the kinase. Mutations that result in increased binding to regulatory proteins might result in constitutive inactivation.

Regulatory domains include short peptide sequences such as those required for nuclear import or export. Mutations in these sequences would result in constitutive cytoplasmic or nuclear localization, respectively, which could either activate or inhibit signaling.

6. Mutations that result in proteins that are capable of binding to an appropriate signaling partner, but the complexes that form are inactive: For example, dimerization of proteins, either homodimers or heterodimers, often is required for signaling; in many instances, short protein sequences are sufficient to promote dimerization. Mutations in functional domains not required for dimerization might result in dominant inhibition; these proteins are capable of binding to and possibly sequestering other signaling molecules into inactive, or partially inactive, complexes.

7. Mutations that decrease or increase the targeting of proteins to the appropriate subcellular destination: Short peptide sequences often facilitate the targeting of proteins to specific subcellular locations. For example, short sequences are sufficient to be recognized and modified by fatty acylation, prenylation, or glycosyl-phosphatidylinositol modification. These modifications result in targeting of proteins to membranes. Membrane spanning peptide sequences also have been identified, as have targeting sequences for secretion. In addition, sequences have been identified that target proteins to subcellular locations such as the endoplasmic reticulum, mitochondria, peroxisome, vacuole, nucleus, and lysosome. Mutations that inhibit proper targeting might result in dominant inhibition; these proteins might be capable of binding to and possibly sequestering other signaling molecules from the appropriate subcellular location.

Mutations that create a new protein function. For example, a mutation in a protein kinase could result in altered substrate specificity, such that the mutated kinase can modulate the activity of pathways that it does not usually regulate.

8. In certain embodiments of the methods according to this aspect of the invention, the modulation is mediated by a peptide modulator of gene expression. The term “peptide” means a molecule comprised of a linear array of amino acid residues connected to each other in the linear array by peptide bonds. Such peptides according to the invention may include from about three to about 500 amino acids, and may further include secondary, tertiary or quaternary structures, as well as intermolecular associations with other peptides or other non-peptide molecules. Such intermolecular associations may be through, without limitation, covalent bonding (e.g., through disulfide linkages), chelation, electrostatic interactions, hydrophobic interactions, hydrogen bonding, ion-dipole interactions, dipole-dipole interactions, or any combination of the above. Peptides may be expressed in the cell or supplied exogenously. Preferably, they are provided on a scaffold to increase intracellular stability and to provide conformational constraint. A “scaffold” is a molecule, most frequently a small protein, from which a peptide is displayed; scaffolds are employed to optimize presentation, rigidity, conformational constraint, and potentially intracellular/extracellular localization. Preferred scaffolds include a catalytically inactive version of staphylococcal nuclease. Preferred peptides according to this aspect of the invention include, without limitation, those peptides disclosed in Norman et al., Science 285: 591-595 (1999).

In certain embodiments of the methods according to this aspect of the invention, the peptide modulator is an activator of gene expression. An “activator of gene expression” is a molecule that causes transcription and/or translation and/or gene product maturation to exceed by at least two-fold, preferably at least five fold, and more preferably at least ten-fold, the level of such expression that would be present under similar growth conditions in the absence of the activator of expression of the gene.

In certain embodiments of the methods according to this aspect of the invention, the peptide modulator is an inhibitor of gene expression. An “inhibitor of gene expression” is a molecule that causes transcription and/or translation and/or gene product maturation to be reduced by at least two-fold, preferably at least five fold, and more preferably at least ten-fold, the level of such expression that would be present under similar growth conditions in the absence of the inhibitor of expression of the gene.

In certain embodiments of the methods according to this aspect of the invention, the modulation is mediated by a small molecule modulator of gene expression. In certain embodiments of the methods according to this aspect of the invention, the small molecule modulator is an activator of gene expression. In certain embodiments of the methods according to this aspect of the invention, the small molecule (i.e., compound with a preferable molecular weight below 1000 daltons) modulator is an inhibitor of gene expression.

In certain embodiments of the methods according to this aspect of the invention, the modulation is conditional expression of the gene. “Conditional expression” of a gene means expression under certain growth conditions, but not under others. Such growth conditions that may be varied include, without limitation, carbon source, nitrogen source, phosphate source, pH, temperature, partial oxygen pressure, the presence or absence of small molecules such as drugs, and the presence or absence of a solid substrate.

In certain embodiments of the methods according to this aspect of the invention, the gene either encodes a transcription factor or the product that it encodes acts on a transcription factor. The term “the gene acts on” means that the gene or its transcriptional, translational, or post-translationally modified product affects the function of its target, preferably by increasing or decreasing transcription, increasing or decreasing translation, increasing or decreasing post-translational modification, increasing or decreasing protein stability, increasing or decreasing protein translocation, or increasing or decreasing protein function by interaction of the protein with another molecule. A “transcription factor” is a molecule that activates or inhibits transcription. The term “activates transcription” means to cause transcription to exceed by at least two-fold, preferably at least five-fold, and more preferably at least ten-fold, the level of transcription that would be present under similar growth conditions in the absence of the transcription factor. The term “inhibits transcription” means to cause transcription to be reduced by at least two-fold, preferably at least five-fold, and more preferably at least ten-fold, the level of such transcription that would be present under similar growth conditions in the absence of the transcription factor. Preferred transcription factors include, without limitation, transcription factors that modulate the expression of genes involved in the production or response to the small molecule cAMP (preferred examples include, without limitation, Mga1, Msn2, Msn4, Sfl1, and Sok2); transcription factors that function downstream of mitogen-activated protein (MAP) kinase signaling pathways that regulate the yeast invasion response (preferred examples include, without limitation, Mcm1, Ste12, and Tec1); transcription factors that modulate the expression of genes involved in nitrogen regulation (preferred examples include, without limitation, AreA, Gln3, Hms1, Hms2, NreB, TamA, and Uga3); transcription factors that modulate the expression of genes involved in pH regulation in fungi (preferred examples include, without limitation PacC and Rim101); general transcription factors (preferred examples include, without limitation, Sin3, Snf2, Srb8, Srb9, Srb10, Srb11, Ssn6, and Tup1); transcription factors that modulate the expression of genes involved in carbon metabolism (preferred examples include, without limitation, Adr1, Cat8, CreA, FacB, Gcr1, Gcr2, Hap4, Mig1, Mig2, Mth1, Nrg1, Oaf1, and Sip4); heme-dependent transcription factors (preferred examples include, without limitation, Hap1 and Rox1); transcription factors that modulate the expression of genes involved in the uptake of metals (preferred examples include, without limitation, Aft1, Cup9, Mac1, SreP, SreA, and Zap 1); transcription factors that modulate the expression of genes involved in cell-cycle regulation (preferred examples include, without limitation, Skn7, Swi4, and Swi6); transcription factors that modulate the expression of genes involved in invasion (preferred examples include, without limitation, Ash1, Flo8, Gts1, Inv7, Msn1, Mss11, Phd1, and Rre1); transcription factors that modulate the expression of genes involved in amino acid biosynthesis or transport (preferred examples include, without limitation, Gcn4, Leu3, Lys14, Met4, Met28, Met31, MetR, Put3, SconB, and Uga3); transcription factors that modulate the expression of genes involved in phosphate metabolism or transport (preferred examples include, without limitation, Pho2 and Pho4); transcription factors that modulate the expression of genes involved in nucleotide metabolism or transport (preferred examples include, without limitation, Ppr1 and UaY); transcription factors that modulate the expression of genes involved in cell wall processes (preferred examples include, without limitation, Ace2, Swi4, and Swi6); transcription factors that modulate the expression of genes involved in sporulation (preferred examples include, without limitation, Ime1 and Ime4); transcription factors that modulate the expression of genes involved in phospholipid synthesis (preferred examples include, without limitation, Ino2); transcription factors that modulate the expression of genes involved in aflatoxin biosynthesis (preferred examples include, without limitation, AflR); transcription factors that modulate the expression of genes involved in lovastatin biosynthesis (preferred examples include, without limitation, AAD34561 and LovE); and transcription factors that modulate the expression of genes involved in filamentous fungal development (preferred examples include, without limitation, AbaA). The term “general transcription factors” means components involved in the formation of preinitiation complexes at promoters that are regulated by RNA polymerase II. The term “invasion” means a process by which a fungus penetrates, digs, adheres to, or attaches to a substrate.

In certain embodiments of the methods according to this aspect of the invention, the gene either encodes a transmembrane transporter or the product that it encodes acts on a transmembrane transporter. A “transmembrane transporter” is a molecule or complex of molecules that facilitates passage of another type of molecule from one side of a cellular membrane to the other side in an energy-dependent or energy-independent manner. “Facilitates passage” means that the number of molecules traversing the membrane is greater than it would have been in the absence of the transmembrane transporter, preferably at least two-fold greater, more preferably at least ten-fold greater, even more preferably at least one hundred-fold greater, and most preferably at least one thousand-fold greater. Preferred classes of transmembrane transporters include, without limitation, proteins of the ATP-binding cassette superfamily, members of the Major Facilitator Superfamily (MFS) that include, without limitation Pump1 and Pump2, P-type ATPases, members of the mitochondrial carrier family (MCF) that include, without limitation, Pet9 and AAD34562, ion channels, permeases that include, without limitation, Bap2, Hip 1, Mep1, and Mep2; and components that transport sugars, ions, or metals.

In certain embodiments of the methods according to this aspect of the invention, the gene either encodes a kinase or the product that it encodes acts on a kinase. A “kinase” is a molecule that phosphorylates a protein, a lipid, a nucleic acid, a carbohydrate, or any other substrate that is capable of being phosphorylated. Preferred kinases include, without limitation, Cdc28, Elm1, Fus3, Gcn2, Hog1, Hsl1, Hxk2, Kss1, Pbs2, Pho85, Rim15, Ste7, Sch9, Snf1, Ste11, Ste20, Tpk1, Tpk2, and Tpk3.

In certain embodiments of the methods according to this aspect of the invention, the gene either encodes a “G-protein” (i.e., guanyl-nucleotide binding protein or the product that it encodes acts on a G-protein. Preferred G-proteins include, without limitation Cdc42, FadA, Gpa1, Gpa2, Ras1, and Ras2.

In certain embodiments of the methods according to this aspect of the invention, the gene either encodes a cell surface receptor or the product that it encodes acts on a cell surface receptor. A “cell surface receptor” is a molecule that resides at the plasma membrane, binds an extracellular signaling molecule, and transduces this signal to propagate a cellular response. Preferred cell surface receptors include, without limitation, G-protein coupled receptors. Preferred G-protein coupled receptors include, without limitation, Gpr1.

In certain embodiments of the methods according to this aspect of the invention, the gene either encodes a GTPase activating protein or the product that it encodes acts on a GTPase activating protein. A “GTPase activating protein” is a molecule that promotes the hydrolysis of GTP bound to a G-protein. GTP-activating proteins often negatively regulate the activity of G-proteins. Preferred GTPase activating proteins include, without limitation, RGS family members. “RGS family members” are regulators of G-protein signaling that act upon G-protein coupled receptors. Preferred RGS family members include, without limitation, FlbA, Rgs2, and Sst2.

In certain embodiments of the methods according to this aspect of the invention, the gene either encodes a guanine nucleotide exchange factor or the product that it encodes acts on a guanine nucleotide exchange factor. A “guanine nucleotide exchange factor” is a molecule that catalyzes the dissociation of GDP from the inactive GTP-binding proteins; following dissociation, GTP can then bind and induce structural changes that activate G-protein signaling. Preferred guanine nucleotide exchange factors include, without limitation, Cdc24 and Cdc25.

In certain embodiments of the methods according to this aspect of the invention, the gene either encodes a phosphatase or the product that it encodes acts on a phosphatase. A “phosphatase” is a molecule that dephosphorylates a protein, a lipid, a nucleic acid, a carbohydrate, or any other substrate that is capable of being dephosphorylated. Preferred phosphatases include, without limitation, Cdc55 and Ptc1.

In certain embodiments of the methods according to this aspect of the invention, the gene either encodes a protease or the product that it encodes acts on a protease. A “protease” is a molecule that cleaves one or more amide bonds in a peptide or protein. “Preferred proteases include, without limitation, Rim13 and LF.

In certain embodiments of the methods according to this aspect of the invention, the gene either encodes a cyclic nucleotide phosphodiesterase or the product that it encodes acts on a cyclic nucleotide phosphodiesterase. A “cyclic nucleotide phosphodiesterase” is a molecule that catalyzes the hydrolysis of the 3′ phosphate bond of a 3′, 5′ cyclic nucleotide to yield free 5′ nucleotide. Preferred examples of cyclic nucleotide phosphodiesterases include, without limitation, Pde2.

In certain embodiments of the methods according to this aspect of the invention, the gene either encodes a bacterial protein toxin or the product that it encodes acts on a bacterial protein toxin. A “bacterial protein toxin” is protein produced by a bacterium, as part of the pathogenesis of the bacterial organism, to kill or impair the biological function of the host organism. Bacterial protein toxins exhibit a wide-variety of biochemical and enzymatic activities including those of adenylate cyclases, ADP-ribosyltransferases, phospholipases, and proteases. Expression of bacterial protein toxins in fungi could result in increased production of secondary metabolites. Preferred bacterial protein toxins include, without limitation, Anthrax toxin edema factor (EF; Bacillus anthracis), Anthrax toxin lethal factor (LF; Bacillus anthracis), adenylate cyclase toxin (Bordetella pertussis), Cholera enterotoxin (Vibrio cholerae), LT toxin (Escherichia coli), ST toxin (E. coli), Shiga toxin (Shigella dysenteriae), Perfringens enterotoxin (Clostridium perfringens), Botulinum toxin (Clostridium botulinum), Tetanus toxin (Clostridium tetani), Diphtheria toxin (Corynebacterium diphtheriae), Exotoxin A (Pseudomonas aeruginosa), Exoenzyme S (P. aeruginosa), Pertussis toxin (Bordetella pertussis), alpha and epsilon toxins (C. perfringens), lethal toxin (LT; Clostridium sordellii), toxins A and B (Clostridium dificile), and phospholipase C (Clostridium bifermentans).

In certain embodiments of the methods according to this aspect of the invention, the gene either encodes an importin protein or the product that it encodes acts on an importin protein. An “importin” protein is a molecule that functions in the translocation of proteins from the nucleus to the cytosol or from the cytosol to the nucleus. Preferred examples of importin proteins include, without limitation, Msn5.

In certain embodiments of the methods according to this aspect of the invention, the gene either encodes a RNA-binding protein or the product that it encodes acts on a RNA-binding protein. Preferred examples of RNA-binding proteins include, without limitation, Dhh1 and Whi3.

In certain embodiments of the methods according to this aspect of the invention, the gene either encodes a component of a SCF complex or the product that it encodes acts on a component of a SCF complex. A “component of a SCF complex” is a molecule in a multi-protein aggregate that targets various substrates involved in the G1 to S phase cell cycle transition for ubiquitin-dependent degradation. Preferred examples of components of a SCF complex include, without limitation, Grr1.

In certain embodiments of the methods according to this aspect of the invention, the gene either encodes or the gene product acts on a biosynthetic enzyme. In certain embodiments of the methods according to this aspect of the invention, the gene acts on biosynthetic enzyme for the secondary metabolite to be produced. RIM9, ROX1, RRE1, SCH9, sconB, SFL1, SHO1, SHR3, SIN3, SIP4, SKN7, SNF1, SNF2, SNF7, SNF8, SOK2, SRB10, SRB11, SRB8, SRB9, sreA, sreP, SRV2, SSD1, SSN6, SST2, STE11, STE12, STE20, STE50, STE7, STP22, SWI4, SWI6, tamA, TEC1, TPK1, TPK2, TPK3, TUP1, UaY, UGA3, URE2, VPS28, VPS36, WHI3, YMR077c, YNL255c, YPR1, ZAP1, genes encoding bacterial protein toxins, and any fungal homologs of the aforementioned genes.

In certain embodiments of the methods according to this aspect of the invention, the methods further comprise purifying the secondary metabolite from a culture of the fungus.

Improving Production of a Secondary Metabolite in a Fungus by Altering the Characteristics of the Fungus in a Manner that is Beneficial to the Production of the Secondary Metabolite

In a fifth aspect, the invention provides methods for improving production of a secondary metabolite in a fungus by altering the characteristics of the fungus in a manner that is beneficial to the production of the secondary metabolite, the method comprising modulating the expression of a gene involved in regulation of secondary metabolite production in a manner that alters the characteristics of the fungus. “Altering the characteristics” means changing the morphology or growth traits of the fungus. Preferred alterations include, without limitation, those alterations that result in transition of the fungus from the hyphal to yeast form, those alterations that result in transition of the fungus from the yeast to hyphal form, alterations that lead to more or less hyphal branching, alterations that increase or decrease flocculence, adherence, cell buoyancy, surface area of the fungus, cell wall integrity and/or stability, pellet size, ability to grow at higher or lower temperatures, and alterations that increase the saturating growth density of a culture or rate of pellet formation.

In certain embodiments of the methods according to this aspect of the invention, the modulation is overexpression of the gene. Preferred genes according to this aspect of the invention include, without limitation, AAD34561, abaA, ACE2, ADR1, AFL1, aflR, AFT1, AGA1, AGA2, amyR, area, ASH1, BAP2, BCY1, BEM1, BEM2, BEM3, BNI1, BUD2, BUD5, CAT8, CDC24, CDC25, CDC28, CDC42, CDC55, CLB2, creA, CTS1, CUP9, CYR1, DFG16, DHH1, DPH3, ELM1, facB, FLO1, FLO10, FLO11, FLO5, FLO8, FLO9, FUS3, GCN2, GCN4, GCR1, GCR2, GIC1, GIC2, GLN3, GPA1, GPA2, GPR1, GRR1, GTS1, HAP1, HAP4, HIP1, HMS1, HMS2, HOG1, HSL1, HXK2, IME1, IME4, INO2, INV11,

In certain embodiments of the methods according to this aspect of the invention, the gene is not AFL1, BEM2, CDC25, DHH1, HOG1, INV11, INV13, INV5, INV7, INV9, IRA1, MCM1, MEP2, MGA1, MSN1, MSN5, MSS1, PET9, PHO23, PTC1, RIM101, RIM13, RIM15, RIM9, SFL1, SNF8, SRB11, SSD1, STP22, SWI4, TPK2, TPK3, VPS28, VPS36, or YPR1. Each of these genes is as described in PCT Publication No. WO99/25865A1

In certain embodiments of the methods according to this aspect of the invention, the gene is selected from the group consisting of AAD34561, AAD34562, abaA, ACE2, ADR1, AFL1, aflR, AFT1, amyR, areA, ASH1, BAP2, BCY1, CAT8, CDC24, CDC25, CDC28, CDC42, CDC55, CLB2, creA, CTS1, CUP9, CYR1, DFG16, DHH1, DPH3, ELM1, facB, FLO1, FLO11, FLO8, FUS3, GCN2, GCN4, GCR1, GCR2, GLN3, GPA1, GPA2, GPR1, GRR1, GTS1, HAP1, HAP4, HIP1, HMS1, HMS2, HOG1, HSL1, HXK2, IME1, IME4, INO2, INV11, INV13, INV16, INV5, INV7, INV9, KSS1, LEU3, lovE, LYS14, MAC1, MCM1, MEP1, MEP2, MET28, MET31, MET4, metR, MGA1, MIG1, MIG2, MSN1, MSN2, MSN4, MSN5, MSS11, MTH1, NPR1, nreB, NRG1, OAF1, pacC, PBS2, PDE2, PET9, PHD1, PHO2, PHO4, PHO85, pkaR, PPR1, PTC1, PUT3, RAS1, RAS2, RGS2, RIM101, RIM13, RIM15, RIM9, ROX1, RRE1, SCH9, sconB, SFL1, SHO1, SHR3, SIN3, SIP4, SKN7, SNF1, SNF2, SNF7, SNF8, SOK2, SRB10, SRB11, SRB8, SRB9, sreA, sreP, SRV2, SSD1, SSN6, SST2, STE11, STE12, STE20, STE50, STE7, STP22, SWI4, SWI6, tamA, TEC1, TPK1, TPK2, TPK3, TUP1, UaY, UGA3, URE2, VPS28, VPS36, WHI3, YMR077c, YNL255c, YPR1, ZAP1, GanB, Gna3, FadA, Gna1, RfeH (PC23), RfeC (An09), lovU, Ste7, Nc1, Vps34, genes encoding bacterial protein toxins, and any fungal homologs of the aforementioned genes. Tables 5 and 6 include nucleic acid sequences for these genes as well as the predicted amino acid sequences of the proteins they encode. Homologs of these genes and proteins from other fungal species are also useful.

A “fungal homolog” of a gene is a gene encoding a gene product that is capable of performing at least a portion of the function of the product encoded by the reference gene, and is substantially identical to the reference gene and/or the encoded product. “Substantially identical” means a polypeptide or nucleic acid exhibiting at least 25%, preferably 50%, more preferably 80%, and most preferably 90%, or even 95% identity to a reference amino acid sequence or nucleic acid sequence. For polypeptides, the length of comparison sequences will generally be at least 16 amino acids, preferably at least 20 amino acids, more preferably at least 25 amino acids, and most preferably 35 amino acids or greater. For nucleic acids, the length of comparison sequences will generally be at least 50 nucleotides, preferably at least 60 nucleotides, more preferably at least 75 nucleotides, and most preferably 110 nucleotides or greater.

Sequence identity is typically measured using sequence analysis software (for example, Sequence Analysis Software Package of the Genetics Computer Group, University of Wisconsin Biotechnology Center, 1710 University Avenue, Madison Wis. 53705, BLAST, BEAUTY, or PILEUP/PRETTYBOX programs). Most preferably, BLAST is used. Such software matches identical or similar sequences by assigning degrees of homology to various substitutions, deletions, and/or other modifications. Conservative substitutions typically include substitutions within the following group: glycine, alanine, valine, isoleucine, leucine; aspartic acid, glutamic acid, asparagine, glutamine; serine, threonine; lysine, arginine; and phenylalanine, tyrosine.

In certain embodiments of the methods according to this aspect of the invention, the methods further comprise purifying the secondary metabolite from a culture of the fungus. “Purifying” means obtaining the secondary metabolite in substantially pure form. “Substantially pure” means comprising at least 90%, more preferably at least 95%, and most preferably at least 99%, of the purified composition on a weight basis.

Improving Production of a Secondary Metabolite by Increasing Productivity of the Secondary Metabolite in the Fungus

In a second aspect, the invention provides methods for improving production of a secondary metabolite by a fungus by increasing productivity of the secondary metabolite in the fungus, the methods comprising modulating the expression of a gene involved in regulation of secondary metabolite production in a manner that improves the productivity of the secondary metabolite. “Increasing productivity” means to increase the quotient for the equation: concentration of secondary metabolite divided by the product of time of fermentor run, fermentation volume, and grams of dry cell weight of biomass (Productivity=concentration metabolite/(time*volume*gDCW)). Significant advantages that might result from increasing productivity include, without limitation, a decrease in fermentor run time, a decrease in the size of fermentor required for production of equivalent amounts of secondary metabolite, or a decrease in the biomass required for production. Collectively, improvements examples include, without limitation, Mcm1, Ste12, and Tec1); transcription factors that modulate the expression of genes involved in nitrogen regulation (preferred examples include, without limitation, AreA, Gln3, Hms1, Hms2, NreB, TamA, and Uga3); transcription factors that modulate the expression of genes involved in pH regulation in fungi (preferred examples include, without limitation PacC and Rim101); general transcription factors (preferred examples include, without limitation, Sin3, Snf2, Srb8, Srb9, Srb10, Srb11, Ssn6, and Tup1); transcription factors that modulate the expression of genes involved in carbon metabolism (preferred examples include, without limitation, Adr1, Cat8, CreA, FacB, Gcr1, Gcr2, Hap4, Mig1, Mig2, Mth1, Nrg1, Oaf1, and Sip4); heme-dependent transcription factors (preferred examples include, without limitation, Hap1 and Rox1); transcription factors that modulate the expression of genes involved in the uptake of metals (preferred examples include, without limitation, Aft1, Cup9, Mac1, SreP, SreA, and Zap1); transcription factors that modulate the expression of genes involved in cell-cycle regulation (preferred examples include, without limitation, Skn7, Swi4, and Swi6); transcription factors that modulate the expression of genes involved in invasion (preferred examples include, without limitation, Ash1, Flo8, Gts1, Inv7, Msn1, Mss11, Phd1, and Rre1); transcription factors that modulate the expression of genes involved in amino acid biosynthesis or transport (preferred examples include, without limitation, Gcn4, Leu3, Lys14, Met4, Met28, Met31, MetR, Put3, SconB, and Uga3); transcription factors that modulate the expression of genes involved in phosphate metabolism or transport (preferred examples include, without limitation, Pho2 and Pho4); transcription factors that modulate the expression of genes involved in nucleotide metabolism or transport (preferred examples include, without limitation, Ppr1 and UaY); transcription factors that modulate the expression of genes involved in cell wall processes (preferred examples include, without limitation, Ace2, Swi4, and Swi6); transcription factors that modulate the expression of genes involved in sporulation (preferred examples include, without limitation, Ime1 and Ime4); transcription factors that modulate the expression of genes involved in phospholipid synthesis (preferred examples include, without limitation, Ino2); transcription factors that modulate the expression of genes involved in aflatoxin biosynthesis (preferred examples include, without limitation, AflR); transcription factors that modulate the expression of genes involved in lovastatin biosynthesis (preferred examples include, without limitation, AAD34561 and LovE); and transcription in productivity can reduce both fixed costs (capital equipment expenses such as fermentor and production facility size, for example) and variable costs (including, but not limited to, decreased waste stream during downstream processing, decreased energy and labor costs, and decreased cost of bulk ingredients). Preferably, such increased productivity is by at least ten percent, more preferably at least 50 percent, and most preferably at least two-fold.

Preferably, for this aspect of the invention, when the secondary metabolite is isopenicillin N, then the modulation is not mediated by the transcription factor CPCR1; when the secondary metabolite is sterigmatocystin, then the modulation is not through AflR, FadA, or FluG; when the secondary metabolite is aflatoxin, then the modulation is not through AflR; when the secondary metabolite is penicillin and the fungus is Aspergillus, then the modulation is not through mutations that result in expression of truncated forms of PacC or constitutively active forms of FadA; when the gene involved in regulation of secondary metabolite production is from Saccharomyces cerevisiae, then the modulation is not through decreased activity or expression of Bem2, Hog1, Ira1, Rim15, Sfl1, Srb11, Ssd1, Swi4, Tpk3 or though increased activity or expression of Afl1, Cdc25, Dhh1, Hap4, Inv11, Inv13, Inv5, Inv7, Inv9, Mcm1, Mep2, Mga1, Msn1, Msn5, Mss11, Pet9, Pho23, Ptc1, Rim101, Rim13, Rim9, Snf8, Stp22, Tpk2, Vps28, Vps36, or Ypr1.

In certain embodiments of the methods according to this aspect of the invention, the modulation is overexpression of the gene. Preferred genes according to this aspect of the invention include, without limitation, AAD34561, AAD34562, abaA, ACE2, ADR1, AFL1, aflR, AFT1, amyR, areA, ASH1, BAP2, BCY1, CAT8, CDC24, CDC25, CDC28, CDC42, CDC55, CLB2, creA, CTS1, CUP9, CYR1, DFG16, DHH1, DPH3, ELM1, facB, FLO1, FLO11, FLO8, FUS3, GCN2, GCN4, GCR1, GCR2, GLN3, GPA1, GPA2, GPR1, GRR1, GTS1, HAP1, HAP4, HIP1, HMS1, HMS2, HOG1, HSL1, HXK2, IME1, IME4, INO2, INV11, INV13, INV16, INV5, INV7, INV9, KSS1, LEU3, lovE, LYS14, MAC1, MCM1, MEP1, MEP2, MET28, MET31, MET4, metR, MGA1, MIG1, MIG2, MSN1, MSN2, MSN4, MSN5, MSS11, MTH1, NPR1, nreB, NRG1, OAF1, pacC, PBS2, PDE2, PET9, PHD1, PHO2, PHO4, PHO85, pkaR, PPR1, PTC1, PUT3, RAS1, RAS2, RGS2, RIM101, RIM13, RIM15, RIM9, ROX1, RRE1, SCH9, sconB, SFL1, SHO1, SHR3, SIN3, SIP4, SKN7, SNF1, SNF2, SNF7, SNF8, SOK2, SRB10, SRB11, SRB8, SRB9, sreA, sreP, SRV2, SSD1, SSN6, SST2, STE11, STE12, STE20, STE50, STE7, STP22, SWI4, SWI6, tamA, TEC1, TPK1, TPK2, factors that modulate the expression of genes involved in filamentous fungal development (preferred examples include, without limitation, AbaA).

In certain embodiments of the methods according to this aspect of the invention, the gene either encodes a transmembrane transporter or the product that it encodes acts on a transmembrane transporter. Preferred classes of transmembrane transporters include, without limitation, proteins of the ATP-binding cassette superfamily, members of the Major Facilitator Superfamily (MFS) that include, without limitation Pump1 and Pump2, P-type ATPases, members of the mitochondrial carrier family (MCF) that include, without limitation, Pet9 and AAD34562, ion channels, permeases that include, without limitation, Bap2, Hip 1, Mep1, and Mep2; and components that transport sugars, ions, or metals.

In certain embodiments of the methods according to this aspect of the invention, the gene either encodes a kinase or the product that it encodes acts on a kinase. Preferred kinases include, without limitation, Cdc28, Elm1, Fus3, Gcn2, Hog1, Hsl1, Hxk2, Kss1, Pbs2, Pho85, Rim15, Ste7, Sch9, Snf1, Ste11, Ste20, Tpk1, Tpk2, and Tpk3.

In certain embodiments of the methods according to this aspect of the invention, the gene either encodes a G-protein or the product that it encodes acts on a G-protein. Preferred G-proteins include, without limitation Cdc42, FadA, Gpa1, Gpa2, Ras1, and Ras2.

In certain embodiments of the methods according to this aspect of the invention, the gene either encodes a cell surface receptor or the product that it encodes acts on a cell surface receptor. Preferred cell surface receptors include, without limitation, G-protein coupled receptors. Preferred G-protein coupled receptors include, without limitation, Gpr1.

In certain embodiments of the methods according to this aspect of the invention, the gene either encodes a GTPase activating protein or the product that it encodes acts on a GTPase activating protein. Preferred GTPase activating proteins include, without limitation, RGS family members. “RGS family members” are regulators of G-protein signaling that act upon G-protein coupled receptors. Preferred RGS family members include, without limitation, FlbA, Rgs2, and Sst2.

In certain embodiments of the methods according to this aspect of the invention, the gene either encodes a guanine nucleotide exchange factor or the product that it encodes acts on a guanine nucleotide exchange factor. Preferred guanine nucleotide exchange factors include, without limitation, Cdc24 and Cdc25. TPK3, TUP1, UaY, UGA3, URE2, VPS28, VPS36, WHI3, YMR077c, YNL255c, YPR1, ZAP1, GanB, Gna3, FadA, Gna1, RfeH (PC23), RfeC (An09), lovU, Ste7, Nc1, Vps34, genes encoding bacterial protein toxins, and any fungal homologs of the aforementioned genes. Tables 5 and 6 include nucleic acid sequences for these genes as well as the predicted amino acid sequences of the proteins they encode. Homologs of these genes and proteins from other fungal species are also useful.

In certain embodiments of the methods according to this aspect of the invention, the modulation is expression of a dominant mutation of the gene.

In certain embodiments of the methods according to this aspect of the invention, the modulation is mediated by a peptide modulator of gene expression. Peptides may be expressed in the cell or supplied exogenously. Preferably, they are provided on a scaffold to increase intracellular stability and to provide conformational constraint.

In certain embodiments of the methods according to this aspect of the invention, the peptide modulator is an activator of gene expression.

In certain embodiments of the methods according to this aspect of the invention, the peptide modulator is an inhibitor of gene expression.

In certain embodiments of the methods according to this aspect of the invention, the modulation is mediated by a small molecule modulator of gene expression. In certain embodiments of the methods according to this aspect of the invention, the small molecule modulator is an activator of gene expression. In certain embodiments of the methods according to this aspect of the invention, the small molecule modulator is an inhibitor of gene expression.

In certain embodiments of the methods according to this aspect of the invention, the modulation is conditional expression of the gene.

In certain embodiments of the methods according to this aspect of the invention, the gene either encodes a transcription factor or the product that it encodes acts on a transcription factor. Preferred transcription factors include, without limitation, transcription factors that modulate the expression of genes involved in the production or response to the small molecule cAMP (preferred examples include, without limitation, Mga1, Msn2, Msn4, Sfl1, and Sok2); transcription factors that function downstream of mitogen-activated protein (MAP) kinase signaling pathways that regulate the yeast invasion response (preferred

In certain embodiments of the methods according to this aspect of the invention, the gene either encodes a phosphatase or the product that it encodes acts on a phosphatase. Preferred phosphatases include, without limitation, Cdc55 and Ptc1.

In certain embodiments of the methods according to this aspect of the invention, the gene either encodes a protease or the product that it encodes acts on a protease. Preferred proteases include, without limitation, Rim13 and LF.

In certain embodiments of the methods according to this aspect of the invention, the gene either encodes a cyclic nucleotide phosphodiesterase or the product that it encodes acts on a cyclic nucleotide phosphodiesterase. Preferred examples of cyclic nucleotide phosphodiesterases include, without limitation, Pde2.

In certain embodiments of the methods according to this aspect of the invention, the gene either encodes a bacterial protein toxin or the product that it encodes acts on a bacterial protein toxin. Preferred bacterial protein toxins include, without limitation, Anthrax toxin edema factor (EF; Bacillus anthracis), Anthrax toxin lethal factor (LF; Bacillus anthracis), adenylate cyclase toxin (Bordetella pertussis), Cholera enterotoxin (Vibrio cholerae), LT toxin (Escherichia coli), ST toxin (E. coli), Shiga toxin (Shigella dysenteriae), Perfringens enterotoxin (Clostridium perfringens), Botulinum toxin (Clostridium botulinum), Tetanus toxin (Clostridium tetani), Diphtheria toxin (Corynebacterium diphtheriae), Exotoxin A (Pseudomonas aeruginosa), Exoenzyme S (P. aeruginosa), Pertussis toxin (Bordetella pertussis), alpha and epsilon toxins (C. perfringens), lethal toxin (LT; Clostridium sordellii), toxins A and B (Clostridium dificile), and phospholipase C (Clostridium bifermentans).

In certain embodiments of the methods according to this aspect of the invention, the gene either encodes an importin protein or the product that it encodes acts on an importin protein. Preferred examples of importin proteins include, without limitation, Msn5.

In certain embodiments of the methods according to this aspect of the invention, the gene either encodes a RNA-binding protein or the product that it encodes acts on a RNA-binding protein. Preferred examples of RNA-binding proteins include, without limitation, Dhh1 and Whi3.

In certain embodiments of the methods according to this aspect of the invention, the gene either encodes a component of a SCF complex or the product that it encodes acts on a component of a SCF complex. Preferred examples of components of a SCF complex include, without limitation, Grr1.

In certain embodiments of the methods according to this aspect of the invention, the gene either encodes or the gene product acts on a biosynthetic enzyme. In certain embodiments of the methods according to this aspect of the invention, the gene acts on biosynthetic enzyme for the secondary metabolite to be produced.

In certain embodiments of the methods according to this aspect of the invention, the gene is selected from the group consisting of AAD34561, AAD34562, abaA, ACE2, ADR1, AFL1, aflR, AFT1, amyR, areA, ASH1, BAP2, BCY1, CAT8, CDC24, CDC25, CDC28, CDC42, CDC55, CLB2, creA, CTS1, CUP9, CYR1, DFG16, DHH1, DPH3, ELM1, facB, FLO1, FLO11, FLO8, FUS3, GCN2, GCN4, GCR1, GCR2, GLN3, GPA1, GPA2, GPR1, GRR1, GTS1, HAP1, HAP4, HIP1, HMS1, HMS2, HOG1, HSL1, HXK2, IME1, IME4, INO2, INV11, INV13, INV16, INV5, INV7, INV9, KSS1, LEU3, lovE, LYS14, MAC1, MCM1, MEP1, MEP2, MET28, MET31, MET4, metR, MGA1, MIG1, MIG2, MSN1, MSN2, MSN4, MSN5, MSS11, MTH1, NPR1, nreB, NRG1, OAF1, pacC, PBS2, PDE2, PET9, PHD1, PHO2, PHO4, PHO85, pkaR, PPR1, PTC1, PUT3, RAS1, RAS2, RGS2, RIM101, RIM13, RIM15, RIM9, ROX1, RRE1, SCH9, SFL1, SHO1, SHR3, SIN3, SIP4, SKN7, SNF1, SNF2, SNF7, SNF8, sconB, SOK2, SRB10, SRB11, SRB8, SRB9, sreA, sreP, SRV2, SSD1, SSN6, SST2, STE11, STE12, STE20, STE50, STE7, STP22, SWI4, SWI6, tamA, TEC1, TPK1, TPK2, TPK3, TUP1, UaY, UGA3, URE2, VPS28, VPS36, WHI3, YMR077c, YNL255c, YPR1, ZAP1, GanB, Gna3, FadA, Gna1, RfeH (PC23), RfeC (An09), lovU, Ste7, Nc1, Vps34, genes encoding bacterial protein toxins, and any fungal homologs of the aforementioned genes. Tables 5 and 6 include nucleic acid sequences for these genes as well as the predicted amino acid sequences of the proteins they encode. Homologs of these genes and proteins from other fungal species are also useful.

In certain embodiments of the methods according to this aspect of the invention, the methods further comprise purifying the secondary metabolite from a culture of the fungus.

Increasing Secondary Metabolite Production by Increasing Efflux or Excretion of the

metabolite

In a third aspect, the invention provides methods for improving production of a secondary metabolite in a fungus by increasing efflux or excretion of the secondary metabolite, the method comprising modulating the expression of a gene involved in regulation of secondary metabolite production in a manner that increases efflux or excretion of the secondary metabolite. “Increasing efflux or excretion of the secondary metabolite” means that a greater quantity of the secondary metabolite passes from the inside of the fungal cells to the outside of the fungal cell per unit time in the absence of lysis of the fungal cells. “Outside of the fungal cell” is defined as being no longer contained wholly within the lipid bilayer of the cell and/or extractable from the cell with methods that do not release a majority of intracellular contents. Increasing efflux of a metabolite could have beneficial impacts on the economics of a fermentation that include, but are not limited to, increasing the amount of metabolite available for isolation in the absence of cell lysis (thus reducing downstream processing costs) and elimination of negative autoregulation by the metabolite to allow increased synthesis.

In certain embodiments of the methods according to this aspect of the invention, the modulation is overexpression of the gene. Preferred genes according to this aspect of the invention include, without limitation, AAD34558, AAD34561, AAD34564, ATR1, ERG6, FCR1, GCN4, lovE, MDR1, PDR1, PDR3, PDR5, PDR10, PDR13, SNQ2, TRI12, and YAP1.

In certain embodiments of the methods according to this aspect of the invention, the modulation is expression of a dominant mutation of the gene.

In certain embodiments of the methods according to this aspect of the invention, the modulation is mediated by a peptide modulator of gene expression. Peptides may be expressed in the cell or supplied exogenously. Preferably, they are provided on a scaffold to increase intracellular stability and to provide conformational constraint. Preferred peptides according to this aspect of the invention include those discussed earlier.

In certain embodiments of the methods according to this aspect of the invention, the peptide modulator is an activator of gene expression.

In certain embodiments of the methods according to this aspect of the invention, the peptide modulator is an inhibitor of gene expression.

In certain embodiments of the methods according to this aspect of the invention, the modulation is mediated by a small molecule modulator of gene expression. In certain embodiments of the methods according to this aspect of the invention, the small molecule modulator is an activator of gene expression. In certain embodiments of the methods according to this aspect of the invention, the small molecule modulator is an inhibitor of gene expression.

In certain embodiments of the methods according to this aspect of the invention, the modulation is conditional expression of the gene. In certain embodiments of the methods according to this aspect of the invention, the gene either encodes a transcription factor or the product that it encodes acts on a transcription factor. Preferred transcription factors include, without limitation, AAD34561, Fcr1, Gcn4, LovE, Pdr1, Pdr3, and Yap1.

In certain embodiments of the methods according to this aspect of the invention, the gene either encodes a transmembrane transporter or the product that it encodes acts on a transmembrane transporter. Preferred transmembrane transporters include, without limitation, AAD34558, AAD34564, Atr1, Mdr1, Pdr5, Pdr10, Snq2, and Tri12.

In certain embodiments of the methods according to this aspect of the invention, the gene either encodes a kinase or the product that it encodes acts on a kinase.

In certain embodiments of the methods according to this aspect of the invention, the gene either encodes a G-protein or the product that it encodes acts on a G-protein.

In certain embodiments of the methods according to this aspect of the invention, the gene either encodes a cell surface receptor or the product that it encodes acts on a cell surface receptor.

In certain embodiments of the methods according to this aspect of the invention, the gene either encodes a GTPase activating protein or the product that it encodes acts on a GTPase activating protein.

In certain embodiments of the methods according to this aspect of the invention, the gene either encodes a guanine nucleotide exchange factor or the product that it encodes acts on a guanine nucleotide exchange factor.

In certain embodiments of the methods according to this aspect of the invention, the gene either encodes a phosphatase or the product that it encodes acts on a phosphatase.

In certain embodiments of the methods according to this aspect of the invention, the gene either encodes a protease or the product that it encodes acts on a protease.

In certain embodiments of the methods according to this aspect of the invention, the gene either encodes a cyclic nucleotide phosphodiesterase or the product that it encodes acts on a cyclic nucleotide phosphodiesterase.

In certain embodiments of the methods according to this aspect of the invention, the gene either encodes a bacterial protein toxin or the product that it encodes acts on a bacterial protein toxin.

In certain embodiments of the methods according to this aspect of the invention, the gene either encodes an importin protein or the product that it encodes acts on an importin protein.

In certain embodiments of the methods according to this aspect of the invention, the gene either encodes an RNA-binding protein or the product that it encodes acts on a RNA-binding protein.

In certain embodiments of the methods according to this aspect of the invention, the gene either encodes a component of a SCF complex or the product that it encodes acts on a component of a SCF complex.

In certain embodiments of the methods according to this aspect of the invention, the gene either encodes or the gene product acts on a biosynthetic enzyme. In certain embodiments of the methods according to this aspect of the invention, the gene acts on biosynthetic enzyme for the secondary metabolite to be produced.

In certain embodiments of the methods according to this aspect of the invention, the gene is selected from the group consisting of AAD34558, AAD34561, AAD34564, ATR1, ERG6, FCR1, GCN4, lovE, MDR1, PDR1, PDR3, PDR5, PDR10, PDR13, SNQ2, TRI12, YAP1, and any fungal homologs of the aforementioned genes.

In certain embodiments of the methods according to this aspect of the invention, the methods further comprise purifying the secondary metabolite from a culture of the fungus.

Improving Production of a Secondary Metabolite in a Fungus by Decreasing Production of Side Products or Non-Desired Secondary Metabolites

In a fourth aspect, the invention provides methods for improving production of a secondary metabolite in a fungus by decreasing production of side products or non-desired secondary metabolites, the method comprising modulating the expression of a gene involved in regulation of secondary metabolite production in a manner that decreases production of side products or non-desired secondary metabolites.

In certain embodiments of the methods according to this aspect of the invention, the modulation is overexpression of the gene.

Preferred genes according to this aspect of the invention include, without limitation, AAD34561, AAD34562, abaA, ACE2, ADR1, AFL1, aflR, AFT1, amyR, areA, ASH1, BAP2, BCY1, CAT8, CDC24, CDC25, CDC28, CDC42, CDC55, CLB2, creA, CTS1, CUP9, CYR1, DFG16, DHH1, DPH3, ELM1, facB, FLO1, FLO11, FLO8, FUS3, GCN2, GCN4, GCR1, GCR2, GLN3, GPA1, GPA2, GPR1, GRR1, GTS1, HAP1, HAP4, HIP1, HMS1, HMS2, HOG1, HSL1, HXK2, IME1, IME4, INO2, INV11, INV13, INV16, INV5, INV7, INV9, KSS1, LEU3, lovE, LYS14, MAC1, MCM1, MEP1, MEP2, MET28, MET31, MET4, metR, MGA1, MIG1, MIG2, MSN1, MSN2, MSN4, MSN5, MSS11, MTH1, NPR1, nreB, NRG1, OAF1, pacC, PBS2, PDE2, PET9, PHD1, PHO2, PHO4, PHO85, pkaR, PPR1, PTC1, PUT3, RAS1, RAS2, RGS2, RIM101, RIM13, RIM15, RIM9, ROX1, RRE1, SCH9, sconB, SFL1, SHO1, SHR3, SIN3, SIP4, SKN7, SNF1, SNF2, SNF7, SNF8, SOK2, SRB10, SRB11, SRB8, SRB9, sreA, sreP, SRV2, SSD1, SSN6, SST2, STE11, STE12, STE20, STE50, STE7, STP22, SWI4, SWI6, tamA, TEC1, TPK1, TPK2, TPK3, TUP1, UaY, UGA3, URE2, VPS28, VPS36, WHI3, YMR077c, YNL255c, YPR1, ZAP1, GanB, Gna3, FadA, Gna1, RfeH (PC23), RfeC (An09), lovU, Ste7, Nc1, Vps34, genes encoding bacterial protein toxins, and any fungal homologs of the aforementioned genes. Tables 5 and 6 include nucleic acid sequences for these genes as well as the predicted amino acid sequences of the proteins they encode. Homologs of these genes and proteins from other fungal species are also useful.

In certain embodiments of the methods according to this aspect of the invention, the modulation is expression of a dominant mutation of the gene.

In certain embodiments of the methods according to this aspect of the invention, the modulation is mediated by a peptide modulator of gene expression.

Peptides may be expressed in the cell or supplied exogenously. Preferably, they are provided on a scaffold to increase intracellular stability and to provide conformational constraint. Preferred peptides according to this aspect of the invention include those discussed earlier.

In certain embodiments of the methods according to this aspect of the invention, the peptide modulator is an activator of gene expression.

In certain embodiments of the methods according to this aspect of the invention, the peptide modulator is an inhibitor of gene expression.

In certain embodiments of the methods according to this aspect of the invention, the modulation is mediated by a small molecule modulator of gene expression. In certain embodiments of the methods according to this aspect of the invention, the small molecule modulator is an activator of gene expression. In certain embodiments of the methods according to this aspect of the invention, the small molecule modulator is an inhibitor of gene expression.

In certain embodiments of the methods according to this aspect of the invention, the modulation is conditional expression of the gene.

In certain embodiments of the methods according to this aspect of the invention, the gene either encodes a transcription factor or the product that it encodes acts on a transcription factor. Preferred transcription factors include, without limitation, transcription factors that modulate the expression of genes involved in the production or response to the small molecule cAMP (preferred examples include, without limitation, Mga1, Msn2, Msn4, Sfl1, and Sok2); transcription factors that function downstream of mitogen-activated protein (MAP) kinase signaling pathways that regulate the yeast invasion response (preferred examples include, without limitation, Mcm1, Ste12, and Tec1); transcription factors that modulate the expression of genes involved in nitrogen regulation (preferred examples include, without limitation, AreA, Gln3, Hms1, Hms2, NreB, TamA, and Uga3); transcription factors that modulate the expression of genes involved in pH regulation in fungi (preferred examples include, without limitation PacC and Rim101); general transcription factors (preferred examples include, without limitation, Sin3, Snf2, Srb8, Srb9, Srb10, Srb11, Ssn6, and Tup1); transcription factors that modulate the expression of genes involved in carbon metabolism (preferred examples include, without limitation, Adr1, Cat8, CreA, FacB, Gcr1, Gcr2, Hap4, Mig1, Mig2, Mth1, Nrg1, Oaf1, and Sip4); heme-dependent transcription factors (preferred examples include, without limitation, Hap1 and Rox1); transcription factors that modulate the expression of genes involved in the uptake of metals (preferred examples include, without limitation, Aft1, Cup9, Mac1, SreP, SreA, and Zap1); transcription factors that modulate the expression of genes involved in cell-cycle regulation (preferred examples include, without limitation, Skn7, Swi4, and Swi6); transcription factors that modulate the expression of genes involved in invasion (preferred examples include, without limitation, Ash1, Flo8, Gts1, Inv7, Msn1, Mss11, Phd1, and Rre1); transcription factors that modulate the expression of genes involved in amino acid biosynthesis or transport (preferred examples include, without limitation, Gcn4, Leu3, Lys14, Met4, Met28, Met31, MetR, Put3, SconB, and Uga3); transcription factors that modulate the expression of genes involved in phosphate metabolism or transport (preferred examples include, without limitation, Pho2 and Pho4); transcription factors that modulate the expression of genes involved in nucleotide metabolism or transport (preferred examples include, without limitation, Ppr1 and UaY); transcription factors that modulate the expression of genes involved in cell wall processes (preferred examples include, without limitation, Ace2, Swi4, and Swi6); transcription factors that modulate the expression of genes involved in sporulation (preferred examples include, without limitation, Ime1 and Ime4); transcription factors that modulate the expression of genes involved in phospholipid synthesis (preferred examples include, without limitation, Ino2); transcription factors that modulate the expression of genes involved in aflatoxin biosynthesis (preferred examples include, without limitation, AflR); transcription factors that modulate the expression of genes involved in lovastatin biosynthesis (preferred examples include, without limitation, AAD34561 and LovE); and transcription factors that modulate the expression of genes involved in filamentous fungal development (preferred examples include, without limitation, AbaA).

In certain embodiments of the methods according to this aspect of the invention, the gene either encodes a transmembrane transporter or the product that it encodes acts on a transmembrane transporter. Preferred classes of transmembrane transporters include, without limitation, proteins of the ATP-binding cassette superfamily, members of the Major Facilitator Superfamily (MFS) that include, without limitation Pump1 and Pump2, P-type ATPases, members of the mitochondrial carrier family (MCF) that include, without limitation, Pet9 and AAD34562, ion channels, permeases that include, without limitation, Bap2, Hip1, Mep1, and Mep2; and components that transport sugars, ions, or metals.

In certain embodiments of the methods according to this aspect of the invention, the gene either encodes a kinase or the product that it encodes acts on a kinase. Preferred kinases include, without limitation, Cdc28, Elm1, Fus3, Gcn2, Hog1, Hsl1, Hxk2, Kss1, Pbs2, Pho85, Rim15, Ste7, Sch9, Snf1, Ste11, Ste20, Tpk1, Tpk2, and Tpk3.

In certain embodiments of the methods according to this aspect of the invention, the gene either encodes a G-protein or the product that it encodes acts on a G-protein. Preferred G-proteins include, without limitation Cdc42, FadA, Gpa1, Gpa2, Ras1, and Ras2.

In certain embodiments of the methods according to this aspect of the invention, the gene either encodes a cell surface receptor or the product that it encodes acts on a cell surface receptor. Preferred cell surface receptors include, without limitation, G-protein coupled receptors. Preferred G-protein coupled receptors include, without limitation, Gpr1.

In certain embodiments of the methods according to this aspect of the invention, the gene either encodes a GTPase activating protein or the product that it encodes acts on a GTPase activating protein. Preferred GTPase activating proteins include, without limitation, RGS family members. Preferred RGS family members include, without limitation, FlbA, Rgs2, and Sst2.

In certain embodiments of the methods according to this aspect of the invention, the gene either encodes a guanine nucleotide exchange factor or the product that it encodes acts on a guanine nucleotide exchange factor. Preferred guanine nucleotide exchange factors include, without limitation, Cdc24 and Cdc25.

In certain embodiments of the methods according to this aspect of the invention, the gene either encodes a phosphatase or the product that it encodes acts on a phosphatase.

Preferred phosphatases include, without limitation, Cdc55 and Ptc1.

In certain embodiments of the methods according to this aspect of the invention, the gene either encodes a protease or the product that it encodes acts on a protease. Preferred proteases include, without limitation, Rim 13 and LF.

In certain embodiments of the methods according to this aspect of the invention, the gene either encodes a cyclic nucleotide phosphodiesterase or the product that it encodes acts on a cyclic nucleotide phosphodiesterase. Preferred examples of cyclic nucleotide phosphodiesterases include, without limitation, Pde2.

In certain embodiments of the methods according to this aspect of the invention, the gene either encodes a bacterial protein toxin or the product that it encodes acts on a bacterial protein toxin. Preferred bacterial protein toxins include, without limitation, Anthrax toxin edema factor (EF; Bacillus anthracis), Anthrax toxin lethal factor (LF; Bacillus anthracis), adenylate cyclase toxin (Bordetella pertussis), Cholera enterotoxin (Vibrio cholerae), LT toxin (Escherichia coli), ST toxin (E. coli), Shiga toxin (Shigella dysenteriae), Perfringens enterotoxin (Clostridium perfringens), Botulinum toxin (Clostridium botulinum), Tetanus toxin (Clostridium tetani), Diphtheria toxin (Corynebacterium diphtheriae), Exotoxin A (Pseudomonas aeruginosa), Exoenzyme S (P. aeruginosa), Pertussis toxin (Bordetella pertussis), alpha and epsilon toxins (C. perfringens), lethal toxin (LT; Clostridium sordellii), toxins A and B (Clostridium dificile), and phospholipase C (Clostridium bifermentans).

In certain embodiments of the methods according to this aspect of the invention, the gene either encodes an importin protein or the product that it encodes acts on an importin protein. Preferred examples of importin proteins include, without limitation, Msn5.

In certain embodiments of the methods according to this aspect of the invention, the gene either encodes a RNA-binding protein or the product that it encodes acts on a RNA-binding protein. Preferred examples of RNA-binding proteins include, without limitation, Dhh1 and Whi3.

In certain embodiments of the methods according to this aspect of the invention, the gene either encodes a component of a SCF complex or the product that it encodes acts on a component of a SCF complex. Preferred examples of components of a SCF complex include, without limitation, Grr1.

In certain embodiments of the methods according to this aspect of the invention, the gene either encodes or the gene product acts on a biosynthetic enzyme. In certain embodiments of the methods according to this aspect of the invention, the gene acts on biosynthetic enzyme for the secondary metabolite to be produced.

In certain embodiments of the methods according to this aspect of the invention, the gene is selected from the group consisting of AAD34561, AAD34562, abaA, ACE2, ADR1, AFL1, aflR, AFT1, amyR, area, ASH1, BAP2, BCY1, CAT8, CDC24, CDC25, CDC28, CDC42, CDC55, CLB2, creA, CTS1, CUP9, CYR1, DFG16, DHH1, DPH3, ELM1, facB, FLO1, FLO11, FLO8, FUS3, GCN2, GCN4, GCR1, GCR2, GLN3, GPA1, GPA2, GPR1, GRR1, GTS1, HAP1, HAP4, HIP1, HMS1, HMS2, HOG1, HSL1, HXK2, IME1, IME4, INO2, INV11, INV13, INV16, INV5, INV7, INV9, KSS1, LEU3, lovE, LYS14, MAC1, MCM1, MEP1, MEP2, MET28, MET31, MET4, metR, MGA1, MIG1, MIG2, MSN1, MSN2, MSN4, MSN5, MSS11, MTH1, NPR1, nreB, NRG1, OAF1, pacC, PBS2, PDE2, PET9, PHD1, PHO2, PHO4, PHO85, pkaR, PPR1, PTC1, PUT3, RAS1, RAS2, RGS2, RIM101, RIM13, RIM15, INV13, INV16, INV5, INV7, INV9, KSS1, LEU3, lovE, LYS14, MAC1, MCM1, MEP1, MEP2, MET28, MET31, MET4, metR, MGA1, MIG1, MIG2, MSN1, MSN2, MSN4, MSN5, MSS11, MTH1, NPR1, nreB, NRG1, OAF1, pacC, PBS2, PDE2, PET9, PHD1, PHO2, PHO4, PHO85, pkaR, PPR1, PTC1, PUT3, RAS1, RAS2, RGA1, RGS2, RHO1, RHO2, RHO3, RHO4, RIM101, RIM13, RIM15, RIM9, ROX1, RRE1, RSR1, SCH9, sconB, SFL1, SHO1, SHR3, SIN3, SIP4, SKN7, SNF1, SNF2, SNF7, SNF8, SOK2, SRB10, SRB11, SRB8, SRB9, sreA, sreP, SRV2, SSD1, SSN6, SST2, STE11, STE12, STE20, STE50, STE7, STP22, SWI4, SWI6, tamA, TEC1, TPK1, TPK2, TPK3, TUP1, UaY, UGA3, URE2, VPS28, VPS36, WHI3, YMR077c, YNL255c, YPR1, ZAP1, GanB, Gna3, FadA, Gna1, RfeH (PC23), RfeC (An09), lovU, Ste7, Nc1, Vps34, genes encoding bacterial protein toxins, and any fungal homologs of the aforementioned genes. Tables 5 and 6 include nucleic acid sequences for these genes as well as the predicted amino acid sequences of the proteins they encode. Homologs of these genes and proteins from other fungal species are also useful.

In certain embodiments of the methods according to this aspect of the invention, the modulation is expression of a dominant mutation of the gene. Preferred dominant mutations according to this aspect of the invention are as used before.

In certain embodiments of the methods according to this aspect of the invention, the modulation is mediated by a peptide modulator of gene expression. Peptides may be expressed in the cell or supplied exogenously. Preferably, they are provided on a scaffold to increase intracellular stability and to provide conformational constraint. Preferred peptides according to this aspect of the invention include those discussed earlier.

In certain embodiments of the methods according to this aspect of the invention, the peptide modulator is an activator of gene expression.

In certain embodiments of the methods according to this aspect of the invention, the peptide modulator is an inhibitor of gene expression.

In certain embodiments of the methods according to this aspect of the invention, the modulation is mediated by a small molecule modulator of gene expression. In certain embodiments of the methods according to this aspect of the invention, the small molecule modulator is an activator of gene expression. In certain embodiments of the methods according to this aspect of the invention, the small molecule modulator is an inhibitor of gene expression.

In certain embodiments of the methods according to this aspect of the invention, the modulation is conditional expression of the gene.

In certain embodiments of the methods according to this aspect of the invention, the gene either encodes a transcription factor or the product that it encodes acts on a transcription factor. Preferred transcription factors include, without limitation, transcription factors that modulate the expression of genes involved in the production or response to the small molecule cAMP (preferred examples include, without limitation, Mga1, Msn2, Msn4, Sfl1, and Sok2); transcription factors that function downstream of mitogen-activated protein (MAP) kinase signaling pathways that regulate the yeast invasion response (preferred examples include, without limitation, Mcm1, Ste12, and Tec1); transcription factors that modulate the expression of genes involved in nitrogen regulation (preferred examples include, without limitation, AreA, Gln3, Hms1, Hms2, NreB, TamA, and Uga3); transcription factors that modulate the expression of genes involved in pH regulation in fungi (preferred examples include, without limitation PacC and Rim101); general transcription factors (preferred examples include, without limitation, Sin3, Snf2, Srb8, Srb9, Srb10, Srb11, Ssn6, and Tup1); transcription factors that modulate the expression of genes involved in carbon metabolism (preferred examples include, without limitation, Adr1, Cat8, CreA, FacB, Gcr1, Gcr2, Hap4, Mig1, Mig2, Mth1, Nrg1, Oaf1, and Sip4); heme-dependent transcription factors (preferred examples include, without limitation, Hap1 and Rox1); transcription factors that modulate the expression of genes involved in the uptake of metals (preferred examples include, without limitation, Aft1, Cup9, Mac1, SreP, SreA, and Zap1); transcription factors that modulate the expression of genes involved in cell-cycle regulation (preferred examples include, without limitation, Skn7, Swi4, and Swi6); transcription factors that modulate the expression of genes involved in invasion (preferred examples include, without limitation, Ash1, Flo8, Gts1, Inv7, Msn1, Mss11, Phd1, and Rre1); transcription factors that modulate the expression of genes involved in amino acid biosynthesis or transport (preferred examples include, without limitation, Gcn4, Leu3, Lys14, Met4, Met28, Met31, MetR, Put3, SconB, and Uga3); transcription factors that modulate the expression of genes involved in phosphate metabolism or transport (preferred examples include, without limitation, Pho2 and Pho4); transcription factors that modulate the expression of genes involved in nucleotide metabolism or transport (preferred examples include, without limitation, Ppr1 and UaY); transcription factors that modulate the expression of genes involved in cell wall processes (preferred examples include, without limitation, Ace2, Swi4, and Swi6); transcription factors that modulate the expression of genes involved in sporulation (preferred examples include, without limitation, Ime1 and Ime4); transcription factors that modulate the expression of genes involved in phospholipid synthesis (preferred examples include, without limitation, Ino2); transcription factors that modulate the expression of genes involved in aflatoxin biosynthesis (preferred examples include, without limitation, AflR); transcription factors that modulate the expression of genes involved in lovastatin biosynthesis (preferred examples include, without limitation, AAD34561 and LovE); and transcription factors that modulate the expression of genes involved in filamentous fungal development (preferred examples include, without limitation, AbaA).

In certain embodiments of the methods according to this aspect of the invention, the gene either encodes a transmembrane transporter or the product that it encodes acts on a transmembrane transporter. Preferred classes of transmembrane transporters include, without limitation, proteins of the ATP-binding cassette superfamily, members of the Major Facilitator Superfamily (MFS) that include, without limitation Pump1 and Pump2, P-type ATPases, members of the mitochondrial carrier family (MCF) that include, without limitation, Pet9, ion channels, permeases that include, without limitation, Bap2, Hip1, Mep1, and Mep2; and components that transport sugars, ions, or metals.

In certain embodiments of the methods according to this aspect of the invention, the gene either encodes a kinase or the product that it encodes acts on a kinase. Preferred kinases include, without limitation, Cdc28, Elm1, Fus3, Gcn2, Hog1, Hsl1, Hxk2, Kss1, Pbs2, Pho85, Rim15, Ste7, Sch9, Snf1, Ste11, Ste20, Tpk1, Tpk2, and Tpk3.

In certain embodiments of the methods according to this aspect of the invention, the gene either encodes a G-protein or the product that it encodes acts on a G-protein. The term Preferred G-proteins include, without limitation Cdc42, FadA, Gpa1, Gpa2, Ras1, Ras2, Rho1, Rho2, Rho3, Rho4, and Rsr1.

In certain embodiments of the methods according to this aspect of the invention, the gene either encodes a cell surface receptor or the product that it encodes acts on a cell surface receptor. Preferred cell surface receptors include, without limitation, G-protein coupled receptors. Preferred G-protein coupled receptors include, without limitation, Gpr1.

In certain embodiments of the methods according to this aspect of the invention, the gene either encodes a GTPase activating protein or the product that it encodes acts on a GTPase activating protein. Preferred GTPase activating proteins include, without limitation, RGS family members. Preferred RGS family members include, without limitation, FlbA, Rgs2, and Sst2. Preferred examples of non-RGS family GTPase-activating proteins include, without limitation, Bem2, Bem3, Bud2, Rga1, and Rga2.

In certain embodiments of the methods according to this aspect of the invention, the gene either encodes a guanine nucleotide exchange factor or the product that it encodes acts on a guanine nucleotide exchange factor. Preferred guanine nucleotide exchange factors include, without limitation, Bud5, Cdc24, and Cdc25.

In certain embodiments of the methods according to this aspect of the invention, the gene either encodes a phosphatase or the product that it encodes acts on a phosphatase. Preferred phosphatases include, without limitation, Cdc55 and Ptc1.

In certain embodiments of the methods according to this aspect of the invention, the gene either encodes a protease or the product that it encodes acts on a protease. Preferred proteases include, without limitation, Rim13 and LF.

In certain embodiments of the methods according to this aspect of the invention, the gene either encodes a cyclic nucleotide phosphodiesterase or the product that it encodes acts on a cyclic nucleotide phosphodiesterase. Preferred examples of cyclic nucleotide phosphodiesterases include, without limitation, Pde2.

In certain embodiments of the methods according to this aspect of the invention, the gene either encodes a bacterial protein toxin or the product that it encodes acts on a bacterial protein toxin. Preferred bacterial protein toxins include, without limitation, Anthrax toxin edema factor (EF; Bacillus anthracis), Anthrax toxin lethal factor (LF; Bacillus anthracis), adenylate cyclase toxin (Bordetella pertussis), Cholera enterotoxin (Vibrio cholerae), LT toxin (Escherichia coli), ST toxin (E. coli), Shiga toxin (Shigella dysenteriae), Perfringens enterotoxin (Clostridium perfringens), Botulinum toxin (Clostridium botulinum), Tetanus toxin (Clostridium tetani), Diphtheria toxin (Corynebacterium diphtheriae), Exotoxin A (Pseudomonas aeruginosa), Exoenzyme S (P. aeruginosa), Pertussis toxin (Bordetella pertussis), alpha and epsilon toxins (C. perfringens), lethal toxin (LT; Clostridium sordellii), toxins A and B (Clostridium dificile), and phospholipase C (Clostridium bifermentans).

In certain embodiments of the methods according to this aspect of the invention, the gene either encodes an importin protein or the product that it encodes acts on an importin protein. Preferred examples of importin proteins include, without limitation, Msn5.

In certain embodiments of the methods according to this aspect of the invention, the gene either encodes a RNA-binding protein or the product that it encodes acts on a RNA-binding protein. Preferred examples of RNA-binding proteins include, without limitation, Dhh1 and Whi3.

In certain embodiments of the methods according to this aspect of the invention, the gene either encodes a component of a SCF complex or the product that it encodes acts on a component of a SCF complex. Preferred examples of components of a SCF complex include, without limitation, Grr1.

In certain embodiments of the methods according to this aspect of the invention, the gene either encodes an adherin or the product that it encodes acts on an adherin. The term “adherin” means a molecule that functions to promote the interaction of a cell with another component, including, without limitation, interaction with other cells of the same genotype, interaction with cells of a different genotype, and interaction with growth substrates. Preferred examples of adherins include, without limitation, Aga1, Aga2, Flo1, Flo10, Flo1, Flo5, and Flo9.

In certain embodiments of the methods according to this aspect of the invention, the gene either encodes or the gene product acts on a biosynthetic enzyme. In certain embodiments of the methods according to this aspect of the invention, the gene acts on biosynthetic enzyme for the secondary metabolite to be produced.

In certain embodiments of the methods according to this aspect of the invention, the gene is selected from the group consisting of AAD34561, abaA, ACE2, ADR1, AFL1, aflR, AFT1, AGA1, AGA2, amyR, areA, ASH1, BAP2, BCY1, BEM1, BEM2, BEM3, BNI1, BUD2, BUD5, CAT8, CDC24, CDC25, CDC28, CDC42, CDC55, CLB2, creA, CTS1, CUP9, CYR1, DFG16, DHH1, DPH3, ELM1, facB, FLO1, FLO10, FLO11, FLO5, FLO8, FLO9, FUS3, GCN2, GCN4, GCR1, GCR2, GIC1, GIC2, GLN3, GPA1, GPA2, GPR1, GRR1, GTS1, HAP1, HAP4, HIP1, HMS1, HMS2, HOG1, HSL1, HXK2, IME1, IME4, INO2, INV1, INV13, INV16, INV5, INV7, INV9, KSS1, LEU3, lovE, LYS14, MAC1, MCM1, MEP1, MEP2, MET28, MET31, MET4, metR, MGA1, MIG1, MIG2, MSN1, MSN2, MSN4, MSN5, MSS11, MTH1, NPR1, nreB, NRG1, OAF1, pacC, PBS2, PDE2, PET9, PHD1, PHO2, PHO4, PHO85, pkaR, PPR1, PTC1, PUT3, RAS1, RAS2, RGA1, RGS2, RHO1, RHO2, RHO3, RHO4, RIM101, RIM13, RIM15, RIM9, ROX1, RRE1, RSR1, SCH9, sconB, SFL1, SHO1, SHR3, SIN3, SIP4, SKI7, SNF1, SNF2, SNF7, SNF8, SOK2, SRB10, SRB11, SRB8, SRB9, sreA, sreP, SRV2, SSD1, SSN6, SST2, STE11, STE12, STE20, STE50, STE7, STP22, SWI4, SWI6, tamA, TEC1, TPK1, TPK2, TPK3, TUP1, UaY, UGA3, URE2, VPS28, VPS36, WHI3, YMR077c, YNL255c, YPR1, ZAP1, GanB, Gna3, FadA, Gna1, RfeH (PC23), RfeC (An09), lovU, Ste7, Nc1, Vps34, genes encoding bacterial protein toxins, and any fungal homologs of the aforementioned genes. Tables 5 and 6 include nucleic acid sequences for these genes as well as the predicted amino acid sequences of the proteins they encode. Homologs of these genes and proteins from other fungal species are also useful. In certain embodiments of the methods according to this aspect of the invention, the methods further comprise purifying the secondary metabolite from a culture of the fungus.

Improving Production of a Secondary Metabolite in a Fungus by Altering the Characteristics of the Fungus in a Manner that is Beneficial to the Production of the Secondary Metabolite

In a sixth aspect, the invention provides methods for improving production of a secondary metabolite in a fungus by altering the characteristics of the fungus in a manner that is beneficial to the production of the secondary metabolite, the method comprising modulating the expression of a gene involved in regulation of secondary metabolite production in a manner that causes conditional lysis. “Causing conditional lysis” means causing the fungus to grow without lysis under a first set of growth conditions and to lyse under a second and different set of conditions, which are not lytic to the unmodified fungus. In preferred embodiments, the conditions that can be altered between the first and second growth conditions include, without limitation, the source or amount of nutrients such as carbon, nitrogen, and phosphate; the source or amount of specific enzymes; the source or amount of specific components found in cell walls; the amount of salts or osmolytes; the pH of the medium, the partial oxygen pressure, or temperature; and the amount of specific small molecules.

In certain embodiments of the methods according to this aspect of the invention, the modulation is overexpression of the gene. Preferred genes according to this aspect of the invention include, without limitation, ACE2, BCK1, BGL2, CHS1, CHS2, CHS3, CTS1, FKS1, GSC2, HOG1, ISR1, KRE6, MID2, MKK1, MKK2, PBS2, PKC1, PPH21, PPH22, PPZ1, PPZ2, PTP2, PTP3, RHO1, RLM1, ROM1, ROM2, SHO1, SKN1, SLG1, SLN1, SLT2, SMP1, SSK1, SSK2, SSK22, STE11, STT3, STT4, SWI4, SWI6, VPS45, WSC2, WSC3, WSC4, and YPD1.

In certain embodiments of the methods according to this aspect of the invention, the modulation is expression of a dominant mutation of the gene. Preferred dominant mutations according to this aspect of the invention are as used before.

In certain embodiments of the methods according to this aspect of the invention, the modulation is mediated by a peptide modulator of gene expression. Peptides may be expressed in the cell or supplied exogenously. Preferably, they are provided on a scaffold to increase intracellular stability and to provide conformational constraint. Preferred peptides according to this aspect of the invention include those discussed earlier.

In certain embodiments of the methods according to this aspect of the invention, the peptide modulator is an activator of gene expression.

In certain embodiments of the methods according to this aspect of the invention, the peptide modulator is an inhibitor of gene expression.

In certain embodiments of the methods according to this aspect of the invention, the modulation is mediated by a small molecule modulator of gene expression. In certain embodiments of the methods according to this aspect of the invention, the small molecule modulator is an activator of gene expression. In certain embodiments of the methods according to this aspect of the invention, the small molecule modulator is an inhibitor of gene expression.

In certain embodiments of the methods according to this aspect of the invention, the modulation is conditional expression of the gene. Among the promoters useful for conditional expression of a gene are the P. chrysogenum xylanase promoter and the A. nidulans xylanase promoter.

In certain embodiments of the methods according to this aspect of the invention, the gene either encodes a transcription factor or the product that it encodes acts on a transcription factor. Preferred transcription factors include, without limitation, Ace2, Rlm1, Smp1, Swi4, and Swi6.

In certain embodiments of the methods according to this aspect of the invention, the gene either encodes a transmembrane transporter or the product that it encodes acts on a transmembrane transporter.

In certain embodiments of the methods according to this aspect of the invention, the gene either encodes a kinase or the product that it encodes acts on a kinase. Preferred kinases include, without limitation, Bck1, Hog1, Isr1, Mkk1, Mkk2, Pbs2, Pkc1, Slt2, Ssk2, and Ssk22.

In certain embodiments of the methods according to this aspect of the invention, the gene either encodes a component involved in cell wall biosynthesis or the product that it encodes acts on a component involved in cell wall biosynthesis. Preferred classes of components involved in cell wall biosynthesis include, without limitation, glucan synthases, glucanases, chitin synthase, and chitinases. Preferred examples of components involved in cell wall biosynthesis include, without limitation, Bgl2, Chs1, Chs2, Chs3, Cts1, Fks1, Gsc2, Kre6, and Skn1.

In certain embodiments of the methods according to this aspect of the invention, the gene either encodes a G-protein or the product that it encodes acts on a G-protein. A “G-protein” is a guanyl-nucleotide binding protein. Preferred G-proteins include, without limitation Rho1.

In certain embodiments of the methods according to this aspect of the invention, the gene either encodes a cell surface receptor or the product that it encodes acts on a cell surface receptor. Preferred cell surface receptors include, without limitation, Sho1 and Sln1.

In certain embodiments of the methods according to this aspect of the invention, the gene either encodes a GTPase activating protein or the product that it encodes acts on a GTPase activating protein.

In certain embodiments of the methods according to this aspect of the invention, the gene either encodes a guanine nucleotide exchange factor or the product that it encodes acts on a guanine nucleotide exchange factor. Preferred guanine nucleotide exchange factors include, without limitation, Rom1 and Rom2.

In certain embodiments of the methods according to this aspect of the invention, the gene either encodes a phosphatase or the product that it encodes acts on a phosphatase. Preferred phosphatases include, without limitation, Pph21, Pph22, Ppz1, Ppz2, Ptp2, Ptp3, and Ptc1.

In certain embodiments of the methods according to this aspect of the invention, the gene either encodes a protease or the product that it encodes acts on a protease.

In certain embodiments of the methods according to this aspect of the invention, the gene either encodes a cyclic nucleotide phosphodiesterase or the product that it encodes acts on a cyclic nucleotide phosphodiesterase.

In certain embodiments of the methods according to this aspect of the invention, the gene either encodes a bacterial protein toxin or the product that it encodes acts on a bacterial protein toxin.

In certain embodiments of the methods according to this aspect of the invention, the gene either encodes an importin or the product that it encodes acts on an importin protein.

In certain embodiments of the methods according to this aspect of the invention, the gene either encodes a RNA-binding protein or the product that it encodes acts on a RNA-binding protein.

In certain embodiments of the methods according to this aspect of the invention, the gene either encodes a component of a SCF complex or the product that it encodes acts on a component of a SCF complex.

In certain embodiments of the methods according to this aspect of the invention, the gene either encodes or the gene product acts on a biosynthetic enzyme. In certain embodiments of the methods according to this aspect of the invention, the gene acts on biosynthetic enzyme for the secondary metabolite to be produced.

In certain embodiments of the methods according to this aspect of the invention, the gene is selected from the group consisting of ACE2, BCK1, BGL2, CHS1, CHS2, CHS3, CTS1, FKS1, GSC2, HOG1, ISR1, KRE6, MID2, MKK1, MKK2, PBS2, PKC1, PPH21, PPH22, PPZ1, PPZ2, PTP2, PTP3, RHO1, RLM1, ROM1, ROM2, SHO1, SKN1, SLG1, SLN1, SLT2, SMP1, SSK1, SSK2, SSK22, STE11, STT3, STT4, SWI4, SWI6, VPS45, WSC2, WSC3, WSC4, YPD1, and fungal homologs of the aforementioned genes.

In certain embodiments of the methods according to this aspect of the invention, the methods further comprise purifying the secondary metabolite from a culture of the fungus.

Improving Production of a Secondary Metabolite in a Fungus by Increasing the Resistance of the Fungus to the Deleterious Effects of Exposure to a Secondary Metabolite

In a seventh aspect, the invention provides methods for improving production of a secondary metabolite in a fungus by increasing the resistance of the fungus to the deleterious effects of exposure to a secondary metabolite made by the same organism, the method comprising modulating the expression of a gene involved in regulation of secondary metabolite production in a manner that increases resistance to the deleterious effects of exposure to a secondary metabolite. “Increasing the resistance of the fungus to the deleterious effects of exposure to a secondary metabolite” means to allow the fungus to survive, grow, or produce secondary metabolite in conditions that otherwise would be toxic or prevent production of secondary metabolite.

In certain embodiments of the methods according to this aspect of the invention, the modulation is overexpression of the gene. Preferred genes according to this aspect of the invention include, without limitation, AAD34558, AAD34561, AAD34564, ATR1, ERG6, ERG11, FCR1, GCN4, lovE, MDR1, PDR1, PDR3, PDR5, PDR11, PDR13, SNQ2, TRI12, YAP1, fungal homologs of the aforementioned genes, and genes that encode β-tubulin, calcineurin (including, without limitation, CNA1), chitin synthase, glucan synthase, HMG CoA reductase, N-terminal aminopeptidases, and RNA polymerase II.

In certain embodiments of the methods according to this aspect of the invention, the modulation is expression of a dominant mutation of the gene. Preferred dominant mutations according to this aspect of the invention are as used before.

In certain embodiments of the methods according to this aspect of the invention, the modulation is mediated by a peptide modulator of gene expression. Peptides may be expressed in the cell or supplied exogenously. Preferably, they are provided on a scaffold to increase intracellular stability and to provide conformational constraint. Preferred peptides according to this aspect of the invention include those discussed earlier.

In certain embodiments of the methods according to this aspect of the invention, the peptide modulator is an activator of gene expression.

In certain embodiments of the methods according to this aspect of the invention, the peptide modulator is an inhibitor of gene expression.

In certain embodiments of the methods according to this aspect of the invention, the modulation is mediated by a small molecule modulator of gene expression. In certain embodiments of the methods according to this aspect of the invention, the small molecule modulator is an activator of gene expression. In certain embodiments of the methods according to this aspect of the invention, the small molecule modulator is an inhibitor of gene expression.

In certain embodiments of the methods according to this aspect of the invention, the modulation is conditional expression of the gene. Among the promoters useful for conditional expression of a gene are the P. chrysogenum xylanase promoter and the A. nidulans xylanase promoter.

In certain embodiments of the methods according to this aspect of the invention, the gene either encodes a transcription factor or the product that it encodes acts on a transcription factor. Preferred transcription factors include, without limitation, AAD34561, Fcr1, Gcn4, LovE, Pdr1, Pdr3, and Yap1.

In certain embodiments of the methods according to this aspect of the invention, the gene either encodes a transmembrane transporter or the product that it encodes acts on a transmembrane transporter. Preferred transmembrane transporters include, without limitation, AAD34558, AAD34564, Atr1, Mdr1, Pdr5, Pdr10, Snq2, and Tri12.

In certain embodiments of the methods according to this aspect of the invention, the gene either encodes a kinase or the product that it encodes acts on a kinase.

In certain embodiments of the methods according to this aspect of the invention, the gene either encodes a G-protein or the product that it encodes acts on a G-protein.

In certain embodiments of the methods according to this aspect of the invention, the gene either encodes a cell surface receptor or the product that it encodes acts on a cell surface receptor.

In certain embodiments of the methods according to this aspect of the invention, the gene either encodes a GTPase activating protein or the product that it encodes acts on a GTPase activating protein.

In certain embodiments of the methods according to this aspect of the invention, the gene either encodes a guanine nucleotide exchange factor or the product that it encodes acts on a guanine nucleotide exchange factor.

In certain embodiments of the methods according to this aspect of the invention, the gene either encodes a phosphatase or the product that it encodes acts on a phosphatase.

In certain embodiments of the methods according to this aspect of the invention, the gene either encodes a protease or the product that it encodes acts on a protease.

In certain embodiments of the methods according to this aspect of the invention, the gene either encodes a cyclic nucleotide phosphodiesterase or the product that it encodes acts on a cyclic nucleotide phosphodiesterase.

In certain embodiments of the methods according to this aspect of the invention, the gene either encodes a bacterial protein toxin or the product that it encodes acts on a bacterial protein toxin.

In certain embodiments of the methods according to this aspect of the invention, the gene either encodes an importin protein or the product that it encodes acts on an importin protein.

In certain embodiments of the methods according to this aspect of the invention, the gene either encodes a RNA-binding protein or the product that it encodes acts on a RNA-binding protein.

In certain embodiments of the methods according to this aspect of the invention, the gene either encodes a component of a SCF complex or the product that it encodes acts on a component of a SCF complex.

In certain embodiments of the methods according to this aspect of the invention, the gene either encodes or the gene product acts on a biosynthetic enzyme. In certain embodiments of the methods according to this aspect of the invention, the gene acts on biosynthetic enzyme for the secondary metabolite to be produced.

In certain embodiments of the methods according to this aspect of the invention, the gene is selected from the group consisting of AAD34558, AAD34561, AAD34564, ATR1, ERG6, ERG11, FCR1, GCN4, lovE, MDR1, PDR1, PDR3, PDR5, PDR10, PDR13, SNQ2, TRI12, YAP1, fungal homologs of the aforementioned genes, and genes that encode beta-tubulin, calcineurin (including, without limitation, CNA1), chitin synthase, glucan synthase, HMG CoA reductase, N-terminal aminopeptidases, and RNA polymerase II.

In certain embodiments of the methods according to this aspect of the invention, the methods further comprise purifying the secondary metabolite from a culture of the fungus.

Genetically Modified Fungi

In an eighth aspect, the invention provides genetically modified fungi, wherein the genetically modified fungi have an ability to produce secondary metabolites and the ability of the genetically modified fungus to produce secondary metabolites has been improved by any of the methods according to the invention.

Methods for Making a Secondary Metabolite

In a ninth aspect, the invention provides a method for making a secondary metabolite, the method comprising culturing a fungus that is genetically modified according to the invention under conditions suitable for the production of secondary metabolites. “Conditions suitable for the production of secondary metabolites” means culture conditions under which the fungus does in fact produce one or more secondary metabolite.

Secondary Metabolites

In certain embodiments of the methods of the invention, the secondary metabolite is an anti-bacterial. An “anti-bacterial” is a molecule that has cytocidal or cytostatic activity against some or all bacteria. Preferred anti-bacterials include, without limitation, β-lactams. Prefererred β-lactams include, without limitation, penicillins and cephalosporins and biosynthetic intermediates thereof. Preferred penicillins and biosynthetic intermediates include, without limitation, isopenicillin N, 6-aminopenicillanic acid (6-APA), penicillin G, penicillin N, and penicillin V. Preferred cephalosporins and biosynthetic intermediates include, without limitation, deacetoxycephalosporin V (DAOC V), deacetoxycephalosporin C (DAOC), deacetylcephalosporin C (DAC), 7-aminodeacetoxycephalosporanic acid (7-ADCA), cephalosporin C, 7-B-(5-carboxy-5-oxopentanamido)-cephalosporanic acid (keto-AD-7ACA), 7-B-(4-carboxybutanamido)-cephalosporanic acid (GL-7ACA), and 7-aminocephalosporanic acid (7ACA).

In certain embodiments of the methods of the invention, the secondary metabolite is an anti-hypercholesterolemic or a biosynthetic intermediate thereof. An “anti-hypercholesterolemic” is a drug administered to a patient diagnosed with elevated cholesterol levels, for the purpose of lowering the cholesterol levels. Preferred anti-hypercholesterolemics include, without limitation, lovastatin, mevastatin, simvastatin, and pravastatin.

In certain embodiments of the methods of the invention, the secondary metabolite is an immunosuppressant or a biosynthetic intermediate thereof. An “immunosuppressant” is a molecule that reduces or eliminates an immune response in a host when the host is challenged with an immunogenic molecule, including immunogenic molecules present on transplanted organs, tissues or cells. Preferred immunosuppressants include, without limitation, members of the cyclosporin family and beauverolide L. Preferred cyclosporins include, without limitation, cyclosporin A and cyclosporin C.

In certain embodiments of the methods of the invention, the secondary metabolite is an ergot alkaloid or a biosynthetic intermediate thereof. An “ergot alkaloid” is a member of a large family of alkaloid compounds that are most often produced in the sclerotia of fungi of the genus Claviceps. An “alkaloid” is a small molecule that contains nitrogen and has basic pH characteristics. The classes of ergot alkaloids include clavine alkaloids, lysergic acids, lysergic acid amides, and ergot peptide alkaloids. Preferred ergot alkaloids include, without limitation, ergotamine, ergosine, ergocristine, ergocryptine, ergocornine, ergotaminine, ergosinine, ergocristinine, ergocryptinine, ergocorninine, ergonovine, ergometrinine, and ergoclavine.

In certain embodiments of the methods of the invention, the secondary metabolite is an inhibitor of angiogenesis or a biosynthetic intermediate thereof. An “angiogenesis inhibitor” is a molecule that decreases or prevents the formation of new blood vessels. Angiogenesis inhibitors have proven effective in the treatment of several human diseases including, without limitation, cancer, rheumatoid arthritis, and diabetic retinopathy. Preferred inhibitors of angiogenesis include, without limitation, fumagillin and ovalicin.

In certain embodiments of the methods of the invention, the secondary metabolite is a glucan synthase inhibitor or a biosynthetic intermediate thereof. A “glucan synthase inhibitor” is a molecule that decreases or inhibits the production of 1,3-β-D-glucan, a structural polymer of fungal cell walls. Glucan synthase inhibitors are a class of antifungal agents. Preferred glucan synthase inhibitors include, without limitation, echinocandin B, pneumocandin B, aculeacin A, and papulacandin.

In certain embodiments of the methods of the invention, the secondary metabolite is a member of the gliotoxin family of compounds or a biosynthetic intermediate thereof. The “gliotoxin family of compounds” are related molecules of the epipolythiodioxopiperazine class. Gliotoxins display diverse biological activities, including, without limitation, antimicrobial, antifungal, antiviral, and immunomodulating activities. Preferred members of the “gliotoxin family of compounds” include, without limitation, gliotoxin and aspirochlorine.

In certain embodiments of the methods of the invention, the secondary metabolite is a fungal toxin or a biosynthetic intermediate thereof. A “fungal toxin” is a compound that causes a pathological condition in a host, either plant or animal. Fungal toxins could be mycotoxins present in food products, toxins produced by phytopathogens, toxins from poisonous mushrooms, or toxins produced by zoopathogens. Preferred fungal toxins include, without limitation, aflatoxins, patulin, zearalenone, cytochalasin, griseofulvin, ergochrome, cercosporin, marticin, xanthocillin, coumarins, tricothecenes, fusidanes, sesterpenes, amatoxins, malformin A, phallotoxins, pentoxin, HC toxin, psilocybin, bufotenine, lysergic acid, sporodesmin, pulcheriminic acid, sordarins, fumonisins, ochratoxin A, and fusaric acid.

In certain embodiments of the methods of the invention, the secondary metabolite is a modulator of cell surface receptor signaling or a biosynthetic intermediate thereof. Modulators of cell surface receptor signaling might function by one of several mechanisms including, without limitation, acting as agonists or antagonists, sequestering a molecule that interacts with a receptor such as a ligand, or stabilizing the interaction of a receptor and molecule with which it interacts. Preferred modulators of cell surface signaling include, without limitation, the insulin receptor agonist L-783,281 and the cholecystokinin receptor antagonist asperlicin.

In certain embodiments of the methods of the invention, the secondary metabolite is a plant growth regulator or a biosynthetic intermediate thereof. A “plant growth regulator” is a molecule that controls growth and development of a plant by affecting processes that include, without limitation, division, elongation, and differentiation of cells. Preferred plant growth regulators include, without limitation, cytokinin, auxin, gibberellin, abscisic acid, and ethylene.

In certain embodiments of the methods of the invention, the secondary metabolite is a pigment or a biosynthetic intermediate thereof. A “pigment” is a substance that imparts a characteristic color. Preferred pigments include, without limitation, melanins and carotenoids.

In certain embodiments of the methods of the invention, the secondary metabolite is an insecticide or a biosynthetic intermediate thereof. An “insecticide” is a molecule that is toxic to insects. Preferred insecticides include, without limitation, nodulisporic acid.

In certain embodiments of the methods of the invention, the secondary metabolite is an anti-neoplastic compound or a biosynthetic intermediate thereof. An “anti-neoplastic” compound is a molecule that prevents or reduces tumor formation. Preferred anti-neoplastic compounds include, without limitation, taxol (paclitaxel) and related taxoids.

### EXAMPLE 1

Preparation of Clones Expressing Regulators of Secondary Metabolite Production

To prepare clones that can be used to genetically modulate the expression of genes involved in secondary metabolism, the following experiments were conducted.

The Gateway (Life Technologies, Inc.) Cloning Technology (U.S. Pat. No. 5,888,732) was used o generate constructs for expression of fungal regulators. The polymerase chain reaction (PCR) was used to amplify cDNA or genomic DNA containing coding sequence for fungal regulators; the resultant PCR products contain common sites at both 5′ and 3′ ends in order to facilitate recombination into the Gateway entry vector MB971 (Life Technologies Inc.). The resultant entry clones were then reacted in a Gateway destination cocktail with plasmid MB1419 (or related destination vectors). MB1419 is derived from pLXZ161. pLXZ161 is a gene vector derived from pBC-phleo (P. Silar, Fungal Genetics Newsletter 42: 73 (1995)) that carries a phleomycin resistance cassette for selection of transformants, as well as a polylinker located between the Aspergillus nidulans PGK promoter and the A. nidulans trpC terminator. pLXZ161 was constructed as follows: First, the Aspergillus nidulans trpC terminator was amplified from A. nidulans genomic DNA by PCR using Turbo Pfu Polymerase as described by the manufacturer (Stratagene, 11011 North Torrey Pines Road, La Jolla, Calif. 92037). Primers used in the reaction are TRPC-1 5′-GCGGCCGCGGCGCCCGGCCCATGTCAACAAGAAT-3′) and TRPC-2 5′-CCGCGGCCGAGTGGAGATGTGGAGT-3′. The resultant product was digested with the restriction enzymes SacII and NotI, purified by agarose gel electrophoresis, and cloned into SacII/NotI-digested pBC-phleo DNA, to generate pLXZ116. Second, the A. nidulans promoter was amplified from A. nidulans genomic DNA by PCR using primers PGK1-1 5′CATGGGGCCCCGTGATGTCTACCTGCCCAC-3′ and PGK1-1 5′-CATGATCGATTGTGGGTAGTTAATGGTATG-3′, Turbo Pfu Polymerase, and reaction conditions as described above. The resultant product was digested with ApaI and ClaI and cloned into ApaI/ClaI-digested pLXZ116, to generate pLXZ161. To produce MB1419, the ccdB (death gene) cassette from pEZC7201 (Life Technologies, Gateway cloning manual) was amplified by PCR using oligos MO511 (GGCCATCGATACAAGTTTGTACAAAAAAGCTGAAC) and MO512 (GCGGCCGCACCACTTTGTACAAGAAAGC), digested with ClaI and NotI, and cloned into NotI/ClaI-digested pLXZ161. This generated a destination vector in which the death gene cassette resides between the A. nidulans PGK promoter and the A. nidulans trpC Terminator of pLXZ161. Thus, destination reactions using this vector allow configuration of any gene in an entry clone to be expressed under the control of the A. nidulans PGK promoter. The fungal selectable marker contained on this plasmid is ble, which confers resistance to phleomycin.

### EXAMPLE 2

Transformation of Aspergillus terreus and Penicillium chrysogenum

Destination clones were transformed into either Aspergillus terreus or Penicillium chrysogenum. In order to transform these fungi, spores were first generated by culture of strain ATTCC#20542 (A. terreus), MF1 (NRRL1951, P. chrysogenum), or MF20 (ATCC#11702, P. chrysogenum) on petri plates containing potato dextrose agar (Difco BRL) at 30° C. for 3-6 days. Spores were removed from PDA either by resuspension in sterile water or Tween-80 (0.1%) or by scraping directly from the plate using a sterile spatula. Yeast extract sucrose medium, or YES (2% Yeast Extract, 6% Sucrose), was inoculated to a density of 1-5×10⁶ spores per ml and incubated with shaking in an Erlenmeyer flask at 26-30° C. for 12-16 hr (250 rpm). Mycelia were harvested by centrifugation at 3200 rpm for 10 minutes, and washed in sterile water two times. Mycelia were resuspended in a filter sterilized solution of Novozyme 234 (Sigma) at 2-5 mg/ml in 1 M MgSO₄ and digested at room temperature with shaking (80 rpm) for 1-2 hr. Undigested material was removed by filtration through Miracloth (Calbiochem, 10394 Pacific Center Court, San Diego, Calif. 92121). After adding 1-2 volumes of STC (0.8 M sorbitol, 25 mm Tris, pH 7.5, and 25 mM CaCl₂), the protoplasts were pelleted by centrifugation at 2500 rpm. Protoplasts were washed 2 times in STC by centrifugation. Resulting protoplasts were resuspended to a density of 5×10⁷ per ml in a solution of STC, SPTC (40% polyethylene glycol in STC) and DMSO in a ratio of 9:1:0.1 and frozen at −80° C. For transformations, two aliquots (100 μl each) of protoplasts were mixed with 1-5 ug of either pBCphleo or destination clones for expression of fungal regulators; mixtures were incubated on ice for 30 min. An aliquot of SPTC (15 μl) was added to each tube and the reaction was incubated at room temperature for 15 minuts. An additional aliquot (500 μl) was added with gentle mixing, and the reaction was incubated for an additional 15 minutes at room temperature. The reaction was next resuspended in 25 ml of molten regeneration medium (Potato Dextrose Agar from Sigma, 3050 Spruce Street, St Louis, Mo. 63103) with 0.8 M sucrose, maintained at 50° C., and poured onto a 150 mm petri plate containing 25 ml of solidified regeneration medium plus phleomycin (60-200 μg/ml for A. terreus and 30 μg/ml for P. chrysogenum). Transformants are typically visible after 2-5 days of incubation at 26-30° C.

Phleomycin resistant colonies were colony purified into small 24 well plates and then examined both on plates and in shake flask cultures. Morphological and developmental effects of the transgene were observed under both growth conditions. Due to the heterogeneous nature of transformation in filamentous fungi, at least 10 (and often many more) phleomycin resistant colonies were pursued. Detailed examination of a subset of phleomycin resistant colonies suggests that approximately 80% of the colonies contain a transgene.

### EXAMPLE 3

Determination of Lovastatin Production

Lovastatin assays were performed using broths from shake flask cultures of A. terreus. A. terreus transformants were grown on modified RPM medium (WO/37629) containing 4% glucose, 0.3% corn steep liquor (Sigma), 0.2% KNO₃, 0.3% KH₂PO₄, 0.05% MgSO₄.7H₂O, 0.05% NaCl, 0.05% polyglycol (Dow), 0.1% trace elements (14.3 g/l ZnSO₄.7H₂O, 2.5 g/l CuSO₄.5H₂O, 0.5 g/l NiCl₂.6H₂O, 13.8 g/l FeSO₄.7H₂O, 8.5 g/l MnSO₄.H₂O, 3 g/l citric acid. H₂O (add first), 1 g/l H₃BO₃, 1 g/l Na₂MoO₄, 2.5 μl CoCl₂.6H₂O). The final pH was adjusted to 6.5. Spores for inoculation were generated by culturing on plates containing minimal medium plus phleomycin for 1 week at 27° C. Spores for shake flask inoculation were removed from plates by dragging the tip of a sterile wooden stick approximately 1 inch across the plate surface. The tip of the stick was then dipped into the shake flask medium and swirled gently. Cultures were grown at 27° C., 225 RPM for 5-6 days.

Quantitative assays were performed to assess the levels of lovastatin in broths from shake flask cultures. To assay lovastatin production, (His)₆HMGCoA reductase was first expressed in Saccharomyces cerevisiae and purified with a nickel column. A. terreus samples were fermented as described above and 0.5 mL samples were taken at day 5-6, put in a 1 mL 96-well plate, and centrifuged to remove mycelia before assaying. Samples were transferred to another 1 mL 96-well plate and frozen at −80° C.

Samples were thawed and 10 μL removed and diluted 1:50 in H₂O. 10 μL of this diluted broth was assayed in a reaction (200 μL total) containing 1 mM L-HMGCoA, 1 mM NADPH, 0.005 mM DTT and 5 μL (His)₆HMGCoA reductase. The disappearance of absorbance at 340 nm was observed over time, and this represents the utilization of NADPH, an electron donor required for the reduction of HMGCoA. Lovastatin inhibits HMGCoA reductase, and thus assays containing lovastatin display a decreased rate of disappearance of absorbance at 340 nm. The initial velocities for NADPH disappearance were calculated for broth-containing samples and reactions containing lovastatin standards. Velocities were then adjusted for dilution, and regression analysis was used to determine metabolite concentration.

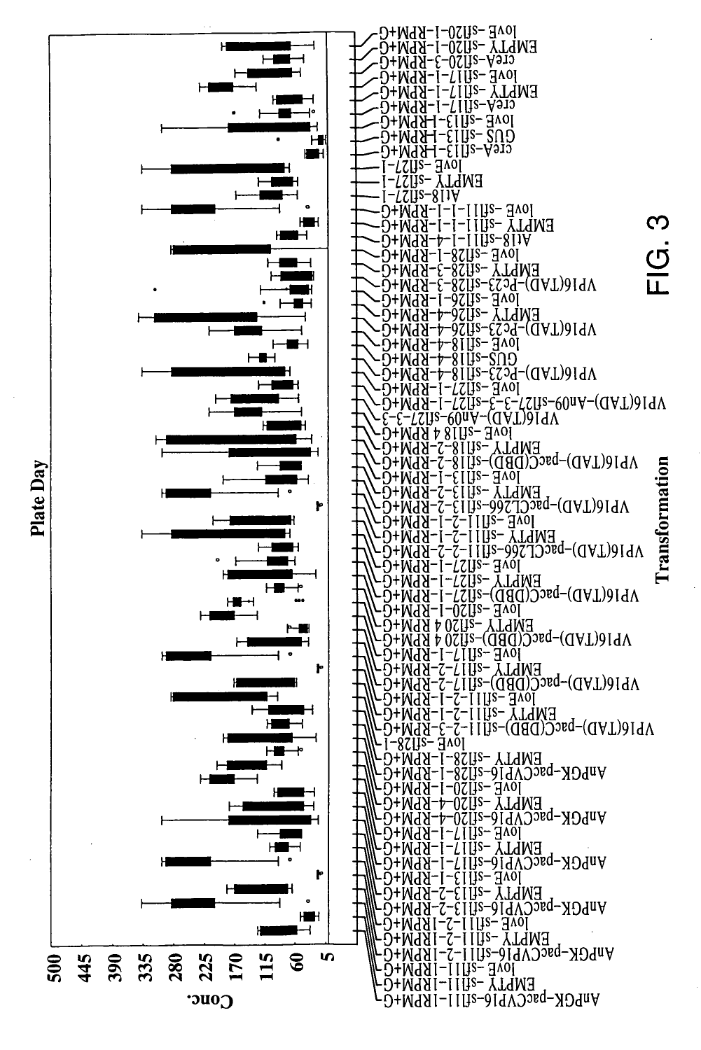

Several fungal regulators were found to improve the overall yield of lovastatin in shake flask cultures. It is possible that these regulators will also increase productivity. Lovastatin production levels from strains containing regulators were compared to either levels from strains containing control vector or a non-transformed strain. Data points were collected for at least 10 phleomycin resistant colonies, and the production levels for each sample set was displayed as a box plot (e.g., FIG. 3). In box plot portrayals of the data, the box represents the central 50% of the data, and the line within the box represents the median value for the entire data set; outlying data points are flagged. Box plot portrayals assist in determining whether a particular sample set is significantly different from a set collected from a control strain.

Hutchinson et al., PCT Publication WO 00/37629, has demonstrated that overexpression of lovE increases lovastatin production in Aspergillus terreus; thus, lovE expressing strains served as positive controls in these experiments. The data in FIG. 3 is organized in sets of three; samples expressing a particular regulator are always compared to control samples (both positive and negative) grown and assayed at the same time. The results in FIG. 3 indicate that several fungal regulators appreciably stimulate production of lovastatin. Table 1 summarizes the results of these studies showing representative fungal regulators that improved the yield of lovastatin in shake flask cultures.

TABLE 1
Representative fungal regulators that increase
lovastatin production in A. terreus

| Plasmid | Regulator |
|---|---|
| MB1423 | pacC (DNA-binding domain (DBD))-VP16 (transcription |
| | activation domain (TAD)) |
| MB1695 | VP16 (TAD)-pacC (DBD) |
| MB1564 | VP16 (TAD)-pacCL266 |
| MB2415 | amdAG229D (TAD)-pacCL266 |
| MB2417 | amdAG229C (TAD)-pacCL266 |
| MB2418 | amdAG229D (TAD)-pacC (DBD) |
| MB2419 | amdAG229D (TAD)-pacC (DBD) |
| MB2203 | VP16 (TAD)-RfeC (An09) |
| MB1316 | lovE |
| MB2244 | VP16 (TAD)-Pc23 |
| MB1970 | At18 |
| MB1310 | creA |

### EXAMPLE 4

Determination of Penicillin Production

Penicillin assays were performed using broths from shake flask cultures of P. chrysogenum. To test levels of penicillin produced in P. chrysogenum transformants, a plug containing spores and mycelia was used as the inoculum. The published P2 production medium (J Lein (1986) in Overproduction of microbial metabolites (Z. Vanek and Z. Hostalek eds.) pp. 105-139), which contains, 30% lactose, 5× pharmamedia cotton seed flour, ammonium sulfate, calcium carbonate, potassium phosphate, potassium sulfate, and phenoxyacetic acid pH 7, was used. Flasks were incubated at 26° C. with shaking at 225 rpm, and sampling was done after 6 days of growth.

To monitor penicillin production, 1-1.5 mls of broth was placed into 96-well plates. The fermentation broth was clarified by centrifugation for 10 min at 4000 g. Supernatants were transferred to a new 96-well plate. Standard samples containing 0, 25, 50, 100, 200, 300, 400, 500 μg/mL phenoxymethylpenicillin (sodium salt) were dissolved in 10 mM potassium phosphate (pH 7.0). For penicillin assays 40 μL of clarified fermentation broth and penicillin standard solutions were transferred to a 96-well UV, collection plate. 200 μL of imidazole reagent was placed in a 96-well filter plate (0.45 micron). The imidazole reagent was prepared by dissolving 8.25 g of imidazole in 60 mL of water, adding 10 mL of 5 M HCl and then adding 10 mL of mercuric chloride solution (0.27 g dissolved in 100 mL of water). The pH of the imidazole reagent was adjusted to 6.80+/−0.05 with 5 M HCl and then diluted to 100 mL with water (see e.g., Bundgaard, H. and K. Ilver, Journal of Pharm Pharmac 24: 790-794 (1972)). The derivatization reaction of penicillin was initiated by vacuum filtration of imidazole reagent into a collection plate containing the aliquoted samples and standards. The collection plate was placed into the 96-well plate reader at 45° C., and an increase at 325 nm was monitored over 20 minutes. A Molecular Devices 96-well UV/is plate reader was used for all spectrophotometric detection.

Several fungal regulators were found to improve the yield of penicillin in shake flask cultures. These experiments were performed in both MFL1951), an early strain in the penicillin development series, and MF20 (ATCC#11702(, a strain of Penicillium chrysogenum that produces approximately ten-fold more penicillin than MF1. As described above for lovastatin, large numbers of phleomycin resistant colonies were used in shake flask experiments, such that analysis could be performed to determine whether the effect of a particular regulator was statistically significant. Strains of MF20 expressing pacCL266 (MB1563), an alkalinity mimicking allege of pacC, displayed increased penicillin production. pacC (DBD)-WP16 (TAD) (e.g., MB1423) stimulated penicillin production in MF1. In addition, both shake flask and small-scale bioreactor studies demonstrate that this regulator can improve the productivity of Penicillium strains; strains expressing pacC (DBD)-WP16 (TAD) initiate production and reach maximum production levels earlier than the parent MF1 strain or a strain transformed with a control vector. Regulators from fungi other than Penicillium chrysogenum also were found to improve penicillin production. Both MF1 and MF20 strains that expressed lovU (MB1317), a gene from Aspergillus terreus, displayed increased yields of penicillin production. Penicillin yields were also improved in MF20 strains that expressed YHR056c, a gene from Saccharomyces cerevisiae.

These results demonstrate that many fungal regulator genes are capable of improving penicillin productions, including genes from unrelated species.

### EXAMPLE 5

Alteration of Fungal Morphology

Fungal morphology can be altered to be favorable to a particular fermentation. Several fungal regulators were found to alter morphological or developmental characteristics of Penicillium strains. Specifically, pacC (DBD)-VP16 (TAD) and VP16 (TAD)-areA (form P. chrysogenum) caused hyphae to aggregate in shake flask cultures. Pellet size is often a crucial factor during growth in bioreactors. Pellet size can impact variables during growth such as the amount of energy needed to drive the impellers within the bioreactor. Aggregating cultures can be beneficial for purification of biomass from culture broth during post-fermentation processing. In addition to these morphological effects, expression of pacC (DBD)-VP16 (TAD), VP16 (TAD)-areA, and VP16-At32 affected the developmental process of sporulation. Strains expressing of pacC (DBD)-VP16 (TAD), VP16(TAD)-areA, At32 (from A. terreus), and VP16-At32 are sporulation defective, whereas strains expressing At32 sporulate in submerged culture. In some instances (e.g., sterigmatocystin biosynthesis in A. nidulans) sporulation and production of secondary metabolites are coordinately regulated. In other examples, such as penicillin production, sporulation defective strains often produce increased levels of metabolite. Therefore, regulators that increase or decrease sporulation may provide a tool to adjust the developmental state of the fungus to the optimal state for production of any particular metabolite

### EXAMPLE 6

Increasing Resistance to the Toxic Effects of a Secondary Metabolite as a Means to Improve Secondary Metabolite Production Including Overexpression of PUMP1 and PUMP2 to Increase Resistance to the Toxic Effects of Lovastatin

Growth of a fungus that produces secondary metabolites can be limited, in part, by the toxic effects of the secondary metabolites themselves. In the absence of resistance mechanisms to protect fungi from toxic effects of these metabolites, decreased yields of the metabolite can be observed. For example, Alexander et al (Mol. Gen. Genet. 261: 977-84 (1999)) have shown that the trichothecene efflux pump of Fusarium sporotrichiodes, encoded by the gene TRI12, is required both for high level production of, and resistance to the toxic effects of, trichothecenes produced by this fungus. Thus, modifications that increase the resistance of a fungus to a toxic secondary metabolite that it produces can increase the saturation density and extend the metabolically active lifetime of the producing fungus. In a bioreactor, such attributes will have the beneficial effect of increasing yield and productivity of a metabolite. Regulators of secondary metabolite production whose expression can be modulated to increase resistance of a fungus to toxic metabolites that it produces can include, without limitation, transporters that promote efflux of the metabolite from cells, enzymes that alter the chemical structure of the metabolite within cells to render it non-toxic, target(s) of the metabolite that mediate its toxicity, and gene products that alter cellular processes to counteract the toxic effects of a metabolite. Additional benefits of increasing efflux of secondary metabolites include increasing the amount of metabolite available for purification from the fermentation broth and mitigation of feedback inhibition of secondary metabolism that may be mediated by the metabolite itself. Indeed, feedback inhibition of a biosynthetic pathway by a product of that pathway is well documented in many microorganisms, and this inhibition can act at the transcriptional, translational, and post-translational levels. Several well-documented examples in yeast include the transcriptional repression of lysine biosynthetic genes by lysine (Feller et al., Eur. J. Biochem. 261: 163-70 (1999)), the decreased stability of both the mRNA encoding the uracil permease Fur4p and the permease itself in the presence of uracil (Seron et al., J. Bacteriol. 181: 1793-800 (1999)), and the inhibition of alpha-isopropyl malate synthase, a key step in leucine biosynthesis, by the presence of leucine (Beltzer et al., J. Biol. Chem. 263: 368-74 (1988)).

Transporters that could mediate resistance to secondary metabolites include members of the major facilitator superfamily (MFS) and the ATP binding cassette (ABC) transporters. For example, overexpression of the class I MFS-type transporter Flr1p in S. cerevisiae has been shown to confer resistance to a variety of toxic compounds such as cycloheximide, fluconazole, 4-nitroquinolone oxide, and cerulenin (Alarco et al., J. Biol. Chem. 272: 19304-13 (1997); Oskouian and Saba, Mol. Gen. Genet. 261: 346-53 (1999)). MFS transporters have been functionally grouped into 23 families in yeast, several of which contain members known or suspected to mediate resistance to toxic compounds by promoting their efflux from the cell (reviewed by Nelissen et al. in FEMS Microbiol. Rev. 21: 113-34 (1997)). Likewise, ABC transporters encoded by genes including PDR5 from S. cerevisiae (Boyum and Guidotti, Biochem. Biophys. Res. Commun. 230: 22-6 (1997)), PMR1 from Penicillium digitatum (Nakuane et al., Appl. Environ. Microbiol. 64: 3983-8 (1998)) and MDR1 from Candida albicans (Sanglard et al., Antimicrob. Agents Chemother. 39: 2378-86 (1995)), amongst others, have been shown to confer resistance to a variety of toxic compounds when their expression is increased. A complete cataloging of ABC transporters in yeast, as well as predicted function based on sequence similarities to transporters of known function, is described in (Decottignies and Goffeau, Nat. Genet. 15: 137-45 (1997)).

Transcription factors that regulate the expression of efflux pumps could also be used to increase efflux of a drug from a fungal cell to increase yields of a metabolite and decrease toxicity of the secondary metabolite in a fermentation. Such transcription factors include, but are not limited to, genes such as YAP1, PDR1, and PDR3 from S. cerevisiae and their homologs. Overexpression of each of these genes has been shown to upregulate expression of transporters and cause increased resistance of S. cerevisiae to toxic compounds (for examples, see Reid et al., J. Biol. Chem. 272: 12091-9 (1997); Katzmann et al., Mol. Cell. Biol. 14: 4653-61 (1994); Wendler et al., J. Biol. Chem. 272: 27091-8 (1997)).

Resistance to the toxic effects of secondary metabolites mediated through modulating expression of target genes will vary with metabolite. For example, amatoxins kill cells by inhibiting the function of the major cellular RNA polymerase, RNA polymerase II, in eucaryotic cells. Mutant forms of RNA polymerase II resistant to the effects of alpha-amanitin have been described (Bartolomei et al., Mol. Cell. Biol. 8: 330-9 (1988); Chen et al., Mol. Cell. Biol. 13: 4214-22 (1993)). Similarly, mutations affecting HMG CoA reductase, the target enzyme for the secondary metabolite lovastatin, have been identified. Increased levels of HMG CoA Reductase can also cause resistance to lovastatin (Ravid et al., J. Biol. Chem. 274: 29341-51 (1999); Lum et al., Yeast 12: 1107-24 (1996)). Taxol (paclitaxel), causes lethality by increasing microtubule stability, thus preventing exit from mitosis. Dominant mutations affecting β-tubulin that confer resistance to taxol have been characterized (for example, see Gonzalez et al., J. Biol. Chem. 274: 23875-82 (1999)) and could prove to be useful to confer resistance of production strains to this toxic metabolite. Such mutatations appear to decrease the stability of microtubules; whether these mutations affect the binding of taxol to microtubules is not known. Similarly, modulating expression of other genes that decrease the stability of microtubules could also confer taxol resistance to a fungus that produces taxol. The pneumocandin and echinocandin families of metabolites are fungal secondary metabolites that inhibit the enzyme 1,3-β-D-glucan synthase. Dominant mutations in the Candida albicans glucan synthase gene, FKS1, have been shown to confer resistance to candins (Douglas et al., Antimicrob. Agents Chemother. 41: 2471-9 (1997)). Glucan synthase mutations such as these could be used to generate fungal production strains with increased resistance to the candin class of antifungals. S. cerevisiae mutants resistant to the growth-inhibitory effects of the fungal secondary metabolite cyclosporin A have also been described (Cardenas et al., EMBO J. 14: 2772-83 (1995)). These mutants were shown to harbor mutations in CNA1, the gene encoding the catalytic subunit of the heterodimeric calcium-calmodulin dependent phosphatase, calcineurin A. Fumagillin, an antiangiogenic agent, binds to and inhibits the N-terminal aminopeptidases in a wide variety of both procaryotes and eucaryotes (Sin et al., Proc. Natl. Acad. Sci. USA 94: 6099-103 (1997), Lowther et al., Proc. Natl. Acad. Sci. USA 95: 12153-7 (1998)). Mutations in this enzyme that block fumagillin binding and/or inhibitory activity could well prove useful in enhancing the resistance of fungal production strains to the growth inhibitory effects of this secondary metabolite.

To demonstrate the feasibility of engineering a fungal strain to be resistant to otherwise toxic amounts of a secondary metabolite, two genes from the lovastatin biosynthetic cluster of A. terreus strain ATCC 20542 were used (Kennedy et al., Science. 284: 1368-72 (1999)). These genes are predicted to encode proteins, denoted by Genbank accession numbers AAD34558 (hereafter referred to as PUMP1) and AAD34564 (hereby referred to as PUMP2), that are members of the MFS class of transporters. As described above, some MFS transporters are known to confer resistance to toxic compounds. PUMP1 and PUMP2 were tested for their ability to confer resistance to otherwise toxic levels of lovastatin when expressed in the fungus S. cerevisiae.

Aspergillus terreus (MF22; ATCC20542) was grown for 45 hours in Production Media at 25° C. (Production Media contains Cerelose, 4.5% (w/v) Peptonized Milk, 2.5% (w/v) Autolyzed yeast, 0.25% (w/v) Polyglycol P2000, 0.25% (w/v) pH to 7.0). Mycelia were harvested in a 50 cc syringe plugged with sterile cotton wool using a vacuum apparatus, washed once with sterile H₂O, and snap frozen in liquid nitrogen. Mycelia were then ground to a powder under liquid nitrogen in a mortar and pestle, and homogenized in RLC buffer (Qiagen RNeasy Kit; Qiagen Inc., 28159 Avenue Stanford, Valencia Calif. 93155) using a GLH rotor-stator homogenizer (Omni International, 6530 Commerce Ct., Suite 100, Warrenton, Va. 20817.) Total RNA was purified using a RNeasy Maxi column according to the instructions of the manufacturer.

The polyA+ fraction of the A. terreus total RNA was isolated using Oligotex beads (Qiagen Inc.). Purified polyA+ RNA (5 μg) was used to generate complementary DNA (cDNA) using Superscript Reverse Transcriptase (Gibco BRL, 9800 Medical Center Drive, PO Box 6482, Rockville, Md. 20849) according to the instructions of the manufacturer. The cDNA was then used to isolate and clone PUMP1 and PUMP2 gene sequences using the polymerase chain reaction (PCR) and Gateway (Life Technologies) Cloning Technology (U.S. Pat. No. 5,888,732). Oligonucleotide sequences used for PCR were 5′-ACAAAAAAGCAGGCTCCACAATGACATCCCACCACGGTGA-3′ (SEQ ID NO: 7) and 5′-ACAAGAAAGCTGGGTTCATTCGCTCCGTCCTTTCT-3′ (SEQ ID NO: 8) for PUMP1. Oligonucleotide sequences used for PUMP2 PCR were 5′-ACAAAAAAGCAGGCTCCACAATGGGCCGCGGTGACACTGA-3′ (SEQ ID NO: 9) and 5′-ACAAGAAAGCTGGGTCTATTGGGTAGGCAGGTTGA-3′ (SEQ ID NO: 10). The resultant plasmids, MB1333 and MB1334, were designed to express PUMP1 and PUMP2, respectively, under control of the S. cerevisiae TEF1 promoter. The plasmids carry a functional URA3 gene to allow for selection of the plasmid on media lacking uracil in a ura3 mutant strain. These plasmids also contained a 2-micron origin for high-copy replication in yeast. Control plasmids were as follows: MB969, the parent vector for MB1333 and MB1334, that does not contain a heterologous gene and is not expected to confer resistance to a yeast strain; MB1344, constructed and described in Donald et al., Appl. Environ. Microbiol. 63: 3341-4 (1997) as pRH127-3, that expresses a soluble form of HMG CoA reductase under control of the yeast GPD1 promoter and is known to confer resistance to increased levels of lovastatin (Donald et al., Appl. Environ. Microbiol. 63: 3341-4 (1997)).

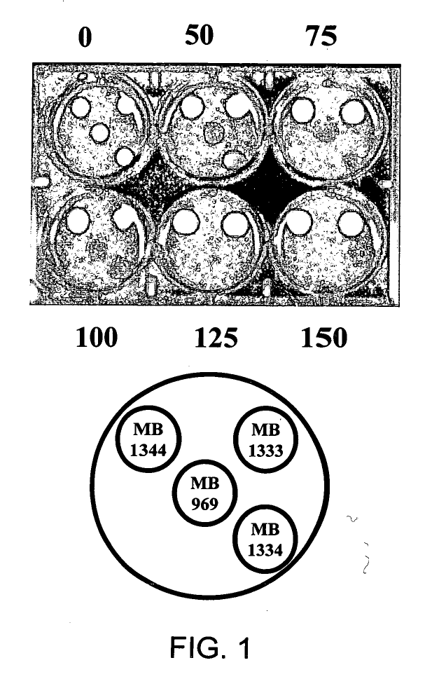

MB1333, MB1334, MB969 and MB1344 were transformed into the yeast strain 22409 (Research Genetics, USA) using standard transformation methods for S. cerevisiae (Biotechniques, 1992, 13(1): 18). Strain 22409 is derived from the S288c strain background of S. cerevisiae, and its complete genotype is as follows: MATa/α, his3Δ1/his3Δ1, leu2Δ0/leu2Δ0, ura3Δ0/ura3Δ0, LYS2/lys2Δ0, MET15/met15Δ0 pdr5::G418/PDR5. Transformants were grown overnight at 30° C. in synthetic complete media lacking uracil (SC-U) to maintain selection for the plasmid. Cultures were diluted 1:10 in sterile water, and 5 μl of each strain was spotted to SC-URA agar containing different concentrations of lovastatin as shown in FIG. 1. Strikingly, the strain harboring MB1333, and thus expressing PUMP1, shows resistance to lovastatin equivalent to the positive control strain in which the soluble fragment of HMG CoA reductase is overexpressed (strain carrying MB1344). These strains show no obvious growth inhibition even at the highest concentrations of lovastatin tested (150 μg/ml). In contrast, the vector-only control and the strain expressing PUMP2 show growth inhibition at the lowest concentration of lovastatin tested (50 μg/mL). Thus, these data indicate that PUMP1 is an excellent candidate for use in engineering lovastatin producing strains to enhance resistance to lovastatin and to promote efflux of this secondary metabolite.

### EXAMPLE 7

Altering Strain Characteristics to Improve Secondary Metabolite Production Including Causing Conditional Lysis

Methods for improving the production of secondary metabolites can involve the construction of strains with desired characteristics for growth or recovery of secondary metabolites. Optimal strain characteristics likely will vary depending upon the fungus being utilized, the particular secondary metabolite being produced, and the specifications of an individual fermentation apparatus. Two traits that might be advantageous for maximal production of secondary metabolites are strains that can be lysed under specific conditions and strains that have morphological characteristics such as increased surface area of active growth and decreased hyphal length. Described below are examples of how both of these traits can be affected by modulating the activity of small GTP-binding proteins (G-proteins).

Fungi must respond to adverse external signals such as osmotic stress. Media for production of secondary metabolites often are hypo-osmotic, whereas fungi that exist on desiccated surfaces must respond to hyper-osmotic stress. One response to hyper-osmotic conditions is to increase the intracellular concentration of osmolytes such as glycerol. During hypo-osmotic stress the integrity of a fungal cell can be maintained both by decreasing intracellular osmolyte concentrations as well as by cell wall modifications. In Saccharomyces cerevisiae the PKC1-SLT2 signaling pathway is required for growth in conditions of low osmolarity (reviewed in Heinisch et al., Mol. Microbiol. 32: 671-680 (1999)). PKC1, which encodes yeast protein kinase C, is activated by components such as the small GTP-binding protein Rho1. Pkc1 then transduces this signal to a MAP kinase signaling cascade that includes the MEK kinase Bck1, the functionally redundant MEKs Mkk1 and Mkk2, and the MAP kinase Slt2. Mutations in genes encoding these signaling components result in varying degrees of cell lysis on media of low osmolarity. Genetic screens have identified many other proteins that function either upstream of PKC1-SLT2 signaling or regulate specific pathway components. These factors include Ppz1, Ppz2, Pph21, Pph22, Ptp2, Ptp3, Isr1, Rom1, Rom2, Mid2, Slg1, Wsc2, Wsc3, Wsc4, Stt3, Stt4, and Vps45; many of these components have homologs in other fungi. In addition, transcription factors, such as Rlm1, Swi4, and Swi6, that can function downstream of PKC1-SLT2 signaling have been identified, and it has been demonstrated that some of these factors are required for the proper expression of genes involved in cell wall biosynthesis. Thus, many components that can modulate the structural integrity of yeast cells have been identified. It is possible that manipulation of these factors could be performed, such that conditional expression of variants of these genes (or the homologs from filamentous fungi) would result in the lysis of fungi and maximal recovery of secondary metabolites.

Conditional lysis of fungi at the conclusion of a fermentor run would be a powerful method for promoting increased recovery of secondary metabolite. Preferably, conditional lysis would require a simple manipulation such as a change in a standard growth parameter (e.g. temperature, dissolved oxygen) or addition of an inexpensive solute. Examples of small molecules that may cause cell lysis include the protein kinase C inhibitor staurosporine, caffeine, dyes that bind the cell wall polymer chitin (e.g. calcofluor white, Congo red), inhibitors of glucan synthase (e.g. candins), and inhibitors of chitin synthase. The cost of using these molecules in a large-scale fermentor likely would be prohibitive. Similarly, addition of enzymes such as glucanases or chitinases would likely be an effective, but costly, method for inducing lysis. An alternative means to induce lysis would be the conditional expression of a dominant negative mutation in a gene encoding a component required for cell wall integrity. Since many components of the PKC1-SLT2 signaling pathway are widely conserved, it is possible that the conditional expression of a dominant inhibitory form of a member of this pathway would facilitate lysis in a variety of fungi, including those fungi that produce secondary metabolites such as lovastatin and cyclosporin A.

The G-protein Rho1 functions to regulate cell wall integrity by at least two independent mechanisms; Rho1 activates Pkc1 signaling as well as 1,3-beta-glucan synthase activity (Nonaka et al., EMBO J. 14: 5931-5938 (1995); Drgonova et al., Science 272: 277-279 (1996); Qadota et al., Science 272: 279-281 (1996)). In addition, dominant inhibitory forms of Rho1 have been identified. Expression of a rho1G22S D125N mutant form in a wild-type Saccharomyces cerevisiae strain results in cell lysis. Therefore, the conditional expression of dominant inhibitory forms of Rho1 under the control of a heat-shock inducible promoter might be an effective method for causing cell lysis in production fungi.

RHO1 coding sequence for construction of dominant mutations can be isolated from Saccharomyces cerevisiae genomic DNA. Primers 5′-cgcGGATCCCGACATATTCGAGGTTGACT-3′ (SEQ ID NO: 11) and 5′-cccAAGCTTGCTAGAAATATGAACCTTCC-3′ (SEQ ID NO: 12) are used to amplify RHO1 coding sequence with 1 kilobase of upstream regulatory sequence and 500 basepairs of downstream regulatory sequence. BamHI and HindIII restriction sites are added to the oligonucleotides to facilitate cloning into the pRS416 centromere-based yeast vector. The Quik Change Site-Directed Mutagenesis Kit (Stratagene, La Jolla Calif.) is used to first create a mutation that encodes the G22S substitution; next, the pRS416rho1G22S plasmid is used as a template to introduce a mutation that encodes the D125N substitution. Primer pair 5′-gtgcctgtAgtaagacatgt-3′/5′-acatgtcttacTacaggcac-3′ is used to anneal to the pRS416RHO1 template for pRS416rho1G22S allele construction. Primer pair 5′-gtaaagtgAatttgagaaac-3′/5′-gtttctcaaatTcactttac-3′ is used to anneal to the pRS416rho1G22S template for pRS416rho1G22S D125N allele construction. pRS416rho1G22S D125N and control plasmids (pRS416RHO1 and pRS416) are then used to transform a wild-type ura3 auxotrophic strain. Transformants are selected and grown at 25° C. in synthetic liquid growth medium lacking uracil and containing the osmolyte sorbitol (1M). Cultures are then transferred to growth in synthetic liquid growth medium lacking uracil without sorbitol, and cells are visually inspected following growth for various periods of time. Expression of the rho1G22S D125N dominant allele causes cell lysis after growth for approximately 120 minutes.

Conditional promoters can be used to express RHO1 dominant mutations in filamentous fungi. The Aspergillus niger tpsB gene is expressed at low levels during growth at ambient temperatures, whereas expression is strongly enhanced upon heat-shock at 40° C.; tpsB regulatory sequence contains multiple copies of the CCCCT stress responsive element (Wolschek et al., J. Biol. Chem. 272: 2729-2735 (1997)). Primers 5′-catgGGGCCCTCTCTCCACCGGCACTAAGATAGC-3′ (SEQ ID NO: 13) and 5′-cgcGGATCCagCATTGGAAAAGGAGGGGGGGGAAG-3′ (SEQ ID NO: 14) are used to amplify 490 basepairs of tpsB upstream regulatory sequence from A. niger genomic DNA. This PCR product contains the tpsB start codon followed by a BamHI cloning site. The tpsB upstream regulatory sequence can be cloned as an ApaI/BamHI fragment into the filamentous fungal vector pLXZ116 (see Example 1). The tpsB promoter is cloned into a multiple cloning site that also contains terminator sequence of the A. nidulans trpC gene. Primers 5′-cgcGGATCCaTCACAACAAGTTGGTAACAGTATC-3′ (SEQ ID NO: 15) and 5′-ggACTAGTTAACAAGACACACTTCTTCTTCTT-3′ (SEQ ID NO: 16) are used to amplify rho1G22SD125N coding sequence, and the product is cloned into the BamHI/SpeI sites of the tpsB containing filamentous fungal vector. This vector can be used to conditionally express (at 40° C.) a dominant negative form of Rho1 that can cause cell lysis.

The filamentous fungal vector containing the tpsB promoter (no RHO1 insert) and a vector containing rho1G22S D125N are used to transform Aspergillus nidulans, Penicillium chrysogenum, and Aspergillus terreus. To assess the impact of conditional expression of a RHO1 dominant negative mutation on cell wall integrity of filamentous fungi, mycelia or spore preps are made from 10 independent transformants, and mycelia or spores are used to inoculate both liquid shake flask cultures and plates containing minimal or rich medium. After growth for 1-2 days the strains are transferred to both 37° C. and 40° C. Strains are examined for morphological defects over the next 24 hours of incubation; potential morphological defects include abnormalities in polarized growth, hyphal wall integrity, and conidiophore development. The optimal time of heat-shock induction required for lysis will be determined. Furthermore, it will be determined whether any abnormalities can be suppressed by growth on medium containing osmotic stabilizers such as sorbitol (1.2 M), sucrose (1 M), or NaCl (1.5 M).

Transformants of Aspergillus terreus that display morphological abnormalities are used to assess whether conditional lysis of strains can be a tool for recovering larger quantities of lovastatin from fermentation broths. Five independent RHO1-containing transformants that display lysis defects will be processed as the A. terreus transformants described in earlier examples. Cultures from each transformant and control strains will be grown for either 8, 9, 10, 11, or 12 days, and cultures will then be incubated at the optimal temperature and for the optimal time required for cell lysis. Following heat shock the cell mass from each culture is separated from the broth by filtration, and the cell mass is lyophilized and weighed. Lovastatin concentration in the broth is calculated as described in earlier examples.

Morphological characteristics such as decreased hyphal length might be advantageous during production of secondary metabolites. For example, strains with shorter filament lengths should display decreased entanglement, floc formation, and shear stress. Such strains would be less susceptible to shear stress damage, these strains might reduce viscosity and facilitate mass transfer, and short filament strains might save energy costs required to power impellers. Increasing the amount of hyphal branching should result in an overall decrease in filament length. The following example describes how expression of a dominant inactive form of the Saccharomyces cerevisiae Rsr1 protein (also known as Bud1) results in increased lateral branch formation.

The yeast Rsr1 protein is required for proper bud site selection; strains lacking Rsr1 bud at random sites on the cell surface. Dominant negative mutations such as rsr1K16N have been identified, and expression of these mutant forms cause random bud site selection without causing obvious growth defects. Expression of rsr1K16N in filamentous fungi may increase branching, decrease filament length, and not have deleterious effects on the growth of the organism.

RSR1 coding sequence for construction of dominant mutations can be isolated from Saccharomyces cerevisiae genomic DNA. Primers 5′-cgcGGATCCTATCTTCACTCAATATACTTCCTA-3′ (SEQ ID NO: 17) and 5′-cccAAGCTTCATCGTTGAAACTTGATAACGCAC-3′ (SEQ ID NO: 18) are used to amplify RHO1 coding sequence with 750 basepairs of upstream regulatory sequence and 500 basepairs of downstream regulatory sequence. BamHI and HindIII restriction sites are added to the oligonucleotides to facilitate cloning into the pRS416 centromere-based yeast vector. The Quik Change Site-Directed Mutagenesis Kit (Stratagene, La Jolla Calif.) is used to create dominant-negative RSR1 substitution mutation K16N. Primer pair 5′-tggtgtcggtaaTtcctgcttaac-3′/5′-gttaagcaggaAttaccgacacca-3′ is used to anneal to the pRS416RSR1 template for allele construction. The pRS416rsr1K16N and control pRS416 plasmids are then used to transform a haploid wild-type ura3 auxotrophic strain. Transformants are selected and grown at 30° C. in YPD liquid growth medium. Log phase cultures are fixed in 3.7% formaldehyde (vol:vol) and stained with the chitin-binding dye Calcofluor white, as described; previous sites of bud formation are marked with a chitin-rich structure called a bud scar. Fluorescent microscopy reveals that cells containing the control plasmid display clustering of bud scars at one pole of the cells, the well-characterized haploid pattern of bud site selection. Cells expressing rsr1K16N display a random pattern of bud site selection; bud scars are scattered across the surface of haploid cells. Cells expressing rsr1K16N do not display other obvious growth or morphological defects.

The Aspergillus nidulans PGK promoter can be used to express RSR1 dominant mutations in filamentous fungi. A filamentous fungal vector containing a multiple cloning site that is flanked by the PGK promoter and terminator sequence of the A. nidulans trpC gene is used. Primers 5′-cgcGGATCCGACTAATGAGAGACTATAAATTAG-3′ (SEQ ID NO: 19) and 5′-ccgCTCGAGCTATAGAATAGTGCAAGTGGAAGC-3′ (SEQ ID NO: 20) are used to amplify rsr1K16N coding sequence, and the product is cloned into the BamHI/XhoI sites of the filamentous fungal vector. This vector can be used to express a dominant negative form of Rsr1 that will affect the process of selecting sites for polarized growth.

The filamentous fungal vector containing rsr1K16N and control vector are used to transform Aspergillus nidulans, Penicillium chrysogenum, and Aspergillus terreus. To assess the impact of expression of RSR1 dominant negative mutations on lateral branch formation and filament length, mycelia and spore preps are made from 10 independent PCR-positive transformants, and mycelia and spores are used to inoculate both liquid shake flask cultures and plates containing minimal or rich medium. Strains are examined at various timepoints over a 48 hour period for morphological alterations, including altered patterns of germ tube emergence, increased lateral branching, decreased filament length, alterations in hyphal width, and changes in chitin staining pattern. Strains displaying desirable morphological changes are then tested in shake flask conditions to determine whether levels of penicillin (A. nidulans, P. chrysogenum) or lovastatin (A. terreus) production have changed significantly.

Aspergillus terreus and Penicillium chrysogenum transformants that display morphological characteristics such as decreased filament length and produce expected or greater levels of lovastatin and penicillin, respectively, are used to assess whether morphological changes can impact upon bioreactor challenges such as shear stress damage, mass transfer, and energy costs. Five independent PCR-positive RSR1-containing transformants that display morphological alterations are grown in a small-scale bioreactor, and examined for improved fermentation characteristics and/or production of secondary metabolite.

### EXAMPLE 8

Genes that Modulate Lovastatin Production

Described below are experiments showing that transcriptional regulators and Gα proteins from diverse fungi can modulate the production of lovastatin in A. terreus. To summarize, transformants expressing CreA and AreA, transcription factors that control carbon and nitrogen metabolism, respectively, showed increased lovastatin production while transformants expressing an activated variant of PacC, a regulator of response to environmental pH, displayed reduced levels of lovastatin. Gα proteins and mutant derivatives thereof that either increased or decreased lovastatin titers also were identified. The results of the experiments described in this Example are summarized in Table 2.

A collection of putative regulators of filamentous fungal secondary metabolite production was generated and screened for specific genes capable of modulating the production of the polyketide lovastatin by A. terreus. Ten transformants were initially analyzed for each expression plasmid to assess effects on lovastatin production. Since the integration site, copy number, and level of transgene expression is expected to vary among transformants, regulators where at least one transformant produced levels of lovastatin that were rarely detected in negative control transformants (α=0.01) were chosen for confirmation and further analysis. These selected expression plasmids were re-transformed, and new isolates were picked and analyzed by HPLC to confirm that the effect on lovastatin production was specific to the transgene. In addition, individual transformants generating increased levels of lovastatin were re-grown and re-assayed to confirm that production levels were reproducible. Statistical significance of the difference between the means of control and transgenic transformants was measured by bootstrap analysis Hall et al., Biometrics 47:757-62 (1991).

Preparation of constructs expressing regulators: Gateway cloning technology (Invitrogen Life Technologies, Inc.) was used to generate all fungal expression constructs using protocols recommended by the manufacturer. Selected genes were amplified in a two-step reaction that resulted in PCR products of the coding region flanked by attB recombination sites. In the first round of PCR, gene specific primers were used with the sequence 5′ ACAAAAAAGCAGGCTCCACA+the first 20 bp of the coding region 3′ and sequence 5′ ACAAGAAAGCTGGT+the complement of the last 20 bp of the coding region (including the stop codon) 3′. For the second round of PCR attB general primers were used: 5′ GGGGACAAGTTTGTACAAAAAAGCAGGCT 3′ and 5′ GGGACCACTTTGTACAAGAAAGCTGGT 3′. The attB site-containing PCR products were then cloned in to the entry clone pDONR206 (pMB971; Invitrogen Life Technologies, Inc.) by a BP reaction and the structure of the resulting “entry clones” was confirmed by sequence analysis. Four “destination vectors” (pMB1419, pMB1473, pMB2957 and pMB3082) were constructed for expression of transgenes. pMB1419 is derived from pBC-phleo (Fungal Genetics Stock Center, Department of Microbiology, University of Kansas Medical Center, Kansas City, Kans.). The A. nidulans pgk promoter was inserted between the ApaI and ClaI sites, and the trpC terminator inserted between NotI and SacII sites. A Gateway cassette containing the appropriate attP sites and the ccdB gene was amplified by PCR using the Gateway vector pEZC7201 as a template (Invitrogen Life Technologies, Inc.) and was ligated into the ClaI and NotI sites. pMB1473 was generated by introducing the sequence encoding the herpes simplex virus VP16 acidic activation domain into the ClaI site of pMB1419 such that introduction of a sequence from an entry clone encoding a transcription factor results in an in-frame N-terminal fusion of the activation domain to the transcription factor of interest. pMB2957 contains the phleomycin resistance marker (ble) transcribed from the trpC promoter, and transgenes are expressed from the A. nidulans fadA promoter. pMB3082 is identical to pMB2957, with the exception that transgenes are expressed from the A. nidulans xlnA promoter (Perez-Gonzalez et al., Appl. Environ. Microbiol. 62:2179-82 (1996). Destination clones in which regulatory genes were cloned into the appropriate expression plasmid were made by performing LR reactions with the desired entry clone and the appropriate destination vector according to protocols recommended by the manufacturer. Site-directed mutations in GÉ subunits were constructed using the QuikChange Site-Directed Mutagenesis Kit (Stratagene).

Transformation of Aspereillus strains: Cultures of A. terreus (Fungal Genetics Stock Center strain 991) or A. nidulans (TJH3.40-Fungal Genetics Stock Center strain A1041) were grown on petri plates containing potato dextrose agar (PDA) (Becton Dickinson) at 30° C. for 3-6 days, and spores were collected by resuspension in Tween-80 (0.03%). Yeast extract sucrose (YES) medium (2% yeast extract, 6% sucrose) with 0.8M sorbitol was inoculated to a density of 1-5×10⁶ spores per ml and incubated with shaking at 26-30° C. for 12-16 hr (250 rpm). Protoplasts were prepared from germlings, frozen and transformed as described Royer et al., Biotechnology 13:1479-83 (1995). Transformation reactions were mixed with 25 ml of molten (50° C.) regeneration minimal medium (trace elements (1000× stock (all g/L)): ZnSO₄ (14.3), CuSO₄ 5H₂O (2.5), NiCl₂ 6H₂O (0.5), FeSO₄ 7H₂O (13.8), MnSO₄H₂O (8.5), citric acid H₂O (3.0), H₃BO₃ (1.0), Na₂MoO₄ (1.0), CoCl₂ 6H₂O (2.5); salts: KCl (0.52 g/L), MgSO₄ (0.52 g/L), KH₂PO₄ (1.52 g/L); 25 mM sodium nitrate, 0.8M sucrose, and 1% agarose at pH 6.5), mixed well and poured onto plates containing 25 ml of regeneration minimal medium with 2× phleomycin, either 60 μg/ml (pMB2957 derivatives) or 300 μg/ml (pMB1419 and pMB1473 derivatives). Transformation plates were incubated at 26° C. for 5-6 days or until colonies started to appear. Transformants were transferred to 12 well plates containing minimal medium (same as regeneration minimal medium but containing 2% instead of 0.8M sucrose) and 1× phleomycin concentration (30 or 150 μg/ml). Plates were incubated at 26° C. for 5-6 days.

Fermentation for production of lovastatin and analysis of lovastatin production: A. terreus transformants and control strains were grown in 20 ml of modified production medium containing 4% glucose, 0.3% corn steep liquor (Sigma), 0.2% KNO₃, 0.3% KH₂PO₄, 0.05% MgSO₄.7H₂O, 0.05% NaCl, 0.05% polyglycol (Dow Chemical Co.), 0.1% trace elements (see above). The final pH was adjusted to 6.5. For xylose induction studies, a mixture of 3% glucose/1% xylose was used as the carbon source. Spores for shake flask inoculation were removed from plates by dragging the tip of a sterile wooden dowel across the plate surface or by using sterile forceps (if not sporulating). Cultures were grown at 27° C. with agitation (225 rpm) for 5 days.

To measure lovastatin production, fermentation broth (0.5 ml) was placed in 1 mL 96-well plates and centrifuged to remove mycelia before assaying. Supernatants were transferred to a second 1 mL 96-well plate and frozen at −80° C. Lovastatin production was assayed using either an HMG-CoA reductase inhibition assay (Dale et al., Eur. J. Biochem. 233:506-13 (1995)) or directly by HPLC. For HPLC analysis, aqueous broths from A. terreus strains were filtered (0.45 μM) to remove particulate matter then diluted 1:6 or 1:10 with a 30% aqueous solution of acetonitrile, depending on expected metabolite concentration. Diluted broths (3 μl) were injected onto a Waters Xterra M-C18 reverse phase HPLC column (2.1×50 mm) at a flow rate of 0.65 mL/min, with column temperature maintained at 55+/−5° C. Gradient elution (mobile phase A: 0.7% (w/w) aqueous acetic acid, mobile phase B: acetonitrile; elution profile is isocratic 58% B for 2.5 minutes, followed by a gradient of 58-90% B for 30 seconds) afforded well-resolved UV detection of open lovastatin (hydrolyzed lactone; 238 nm, retention time of 2.5 min). An authentic sample of lovastatin (Sigma) was used to generate a standard integration curve, enabling absolute quantification of lovastatin.

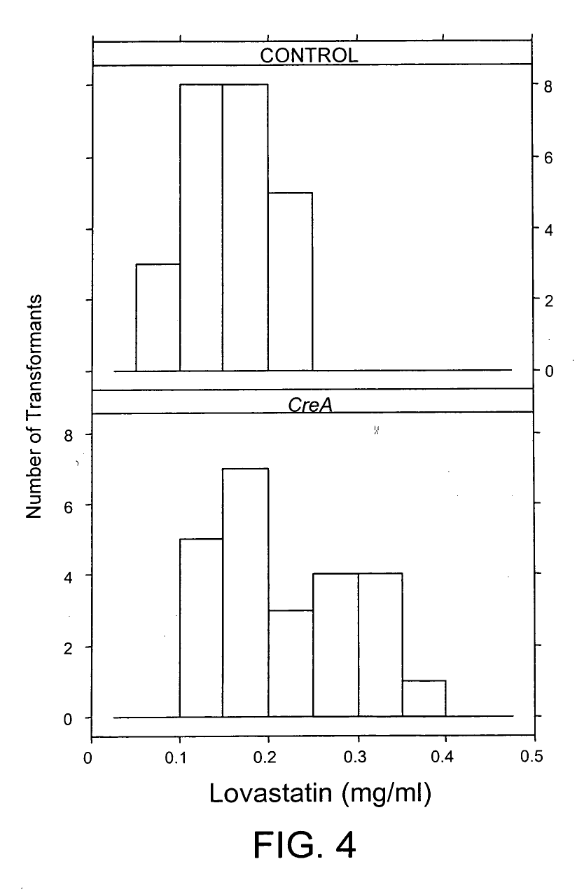

A. nidulans CreA increases lovastatin production by A. terreus: Transformation of A. terreus FGSC 991 with an expression plasmid for A. nidulans CreA, the carbon catabolite repressor protein (Dowzer et al., Curr. Genet. 15:457-59 (1989)), led to increased lovastatin production (FIG. 4). Nine of 24 creA transformants had lovastatin values (greater than 0.25 mg/ml lovastatin) outside the control range and the populations had significantly different means (p<0.01).

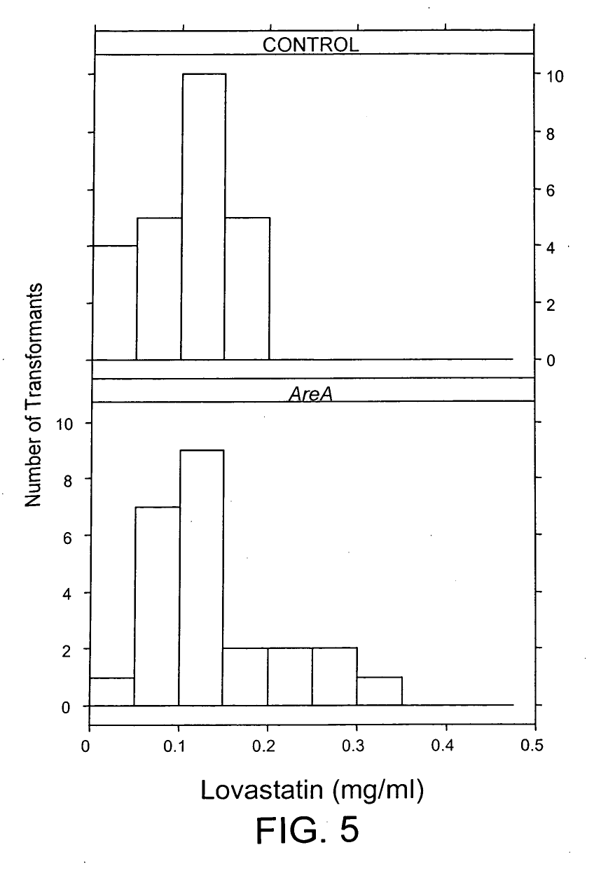

P. chrysogenum areA increases lovastatin production by A. terreus: Transformation of A. terreus FGSC 991 with an expression plasmid for of P. chrysogenum areA, which encodes a regulator of nitrogen metabolism (Haas et al., Curr. Genet. 27:150-58 (1995)), led to transformants with increased levels of lovastatin production, although to a lesser extent than creA transformants (FIG. 5).

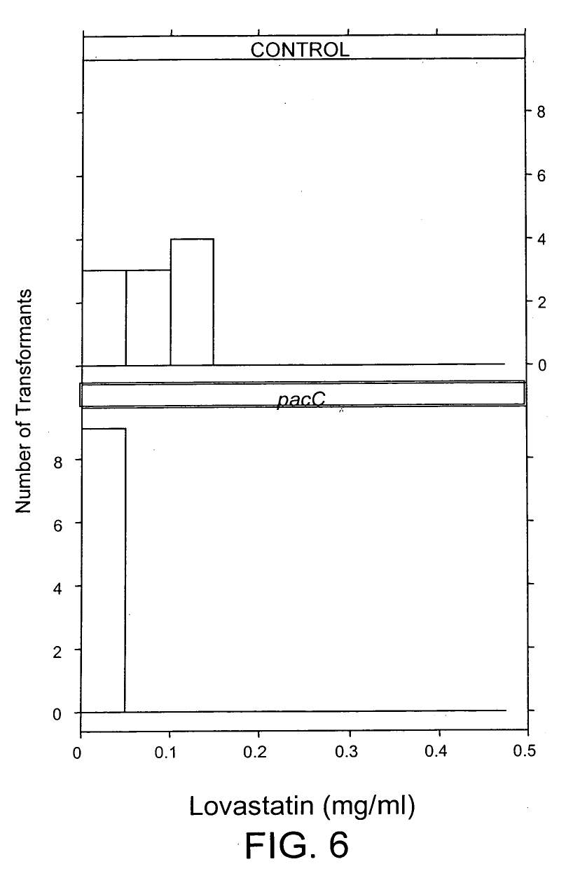

Constitutively active truncation deletion mutants of the A. nidulans pacC decrease lovastatin production by A. terreus: Transformants containing a constitutively active truncation allele of the A. nidulans pacC gene, a regulator of pH-controlled genes (Denison, Fungal Genet. Biol. 29:61-71 (2000); Orejas et al., Genes Dev. 9:1622-32 (1995); Tilbum et al., EMBO J. 14:779-90 (1995)) produced greatly reduced levels of lovastatin (FIG. 6).

The α subunit of a heterotrimeric G proteins modulates lovastatin production in A. terreus: We examined the effect of expression of FadA (A. nidulans) and 7 other fungal Gα proteins, Gpa1 (Ustilago maydis), Gna1 (Neurospora crassa), GanB (A. nidulans), Gna3 (N. crassa), Gpa2 (U. maydis), GanA (A. nidulans), and Gna2 (N. crassa) on lovastatin production. Based on structural conservation, we also generated dominant-activated alleles of each Gα protein by site-directed mutagenesis (Gpa1^{G42R}, Gna1^{G42R}, Gpa1^{Q204L}, FadA^{G42R}, GanB^{G45R}, Gna3^{G44R}, Gpa2^{G45R}, GanA^{G47R}, and Gna2^{G43R}).

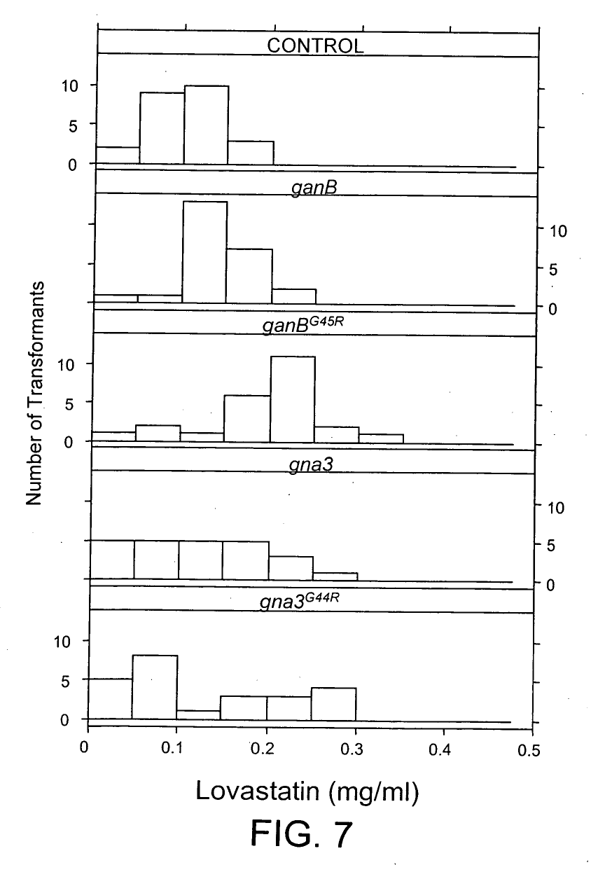

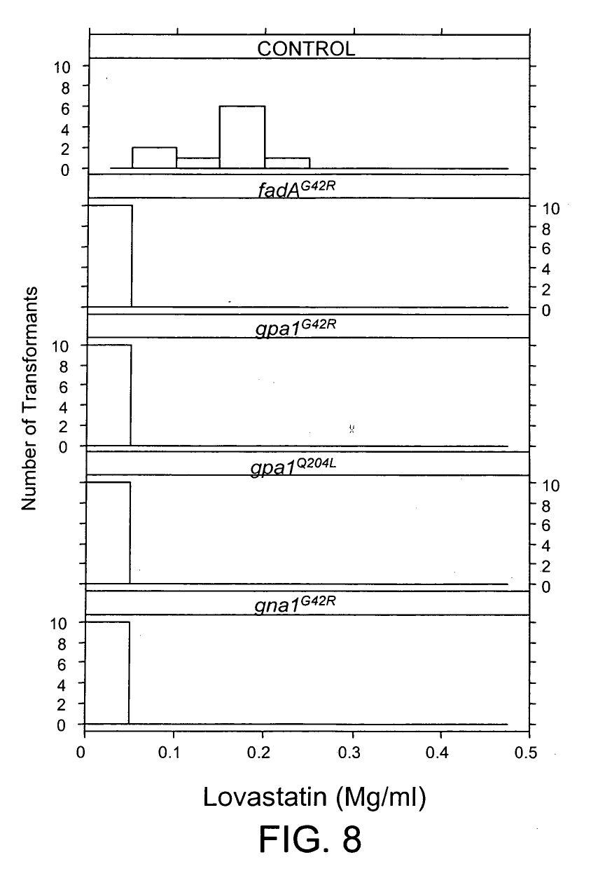

A. terreus FGSC 991 transformed with either wild-type ganB or an activated allele (ganB^{G45R}) resulted in strains that produced significantly more lovastatin than control transformants (p<0.01) (FIG. 7). Transformation with expression plasmids containing wild-type gna3 and an activated point mutation (gna3^{G44R}) led to individual transformants that showed elevated expression of lovastatin (FIG. 7). In contrast, several Gα subunits depressed lovastatin production, but only when activated forms were expressed. Expression of Gα proteins from A. nidulans (FadA^{G42R}), N. crassa (Gna1^{G42R) and} U. maydis (Gpa1^{G42R}, Gpa1^{Q204L}) abolished lovastatin production in 5-day cultures of transformants (FIG. 8). Transformants expressing either wild-type or activated forms of U maydis Gpa2, A. nidulans GanA, and N. crassa Gna2 from the fadA promoter did not display significantly increased or reduced lovastatin titers (data not shown).

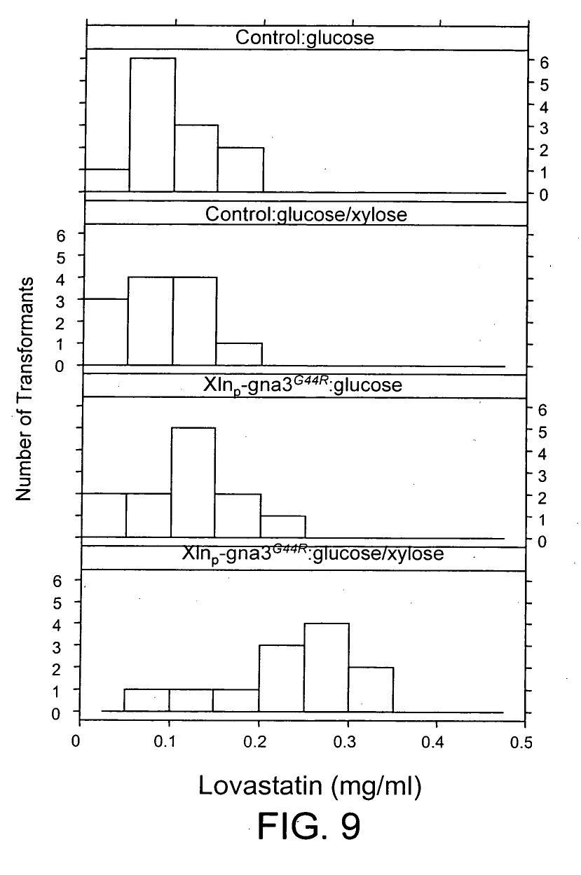

To confirm that the moderate enhancement of lovastatin production in gna3^{G44R} transformants was directly related to the expression of the gene, we expressed Gna3^{G44R} under the control of the xylose-inducible xlnA promoter of A. nidulans Perez-Gonzalez et al., Appl. Environ. Microbiol. 62:2179-82 (1996). Transformants containing xlnAp-gna3^{G44R} showed a clear enhancement in lovastatin production in inducing medium (p<0.01), whereas neither the negative control transformants nor the fadAp-gna3^{G44R} transformants were significantly affected by carbon source (FIG. 9 and data not shown).

### EXAMPLE 9

Identification of Additional Genes that Modulate Lovastatin Production

In order to identify additional genes that could influence secondary metabolism, we established a functional screen for regulators of fungal physiology based on the regulatory region of the S. cerevisiae FLO11 gene. The expression of Flo11, a cell surface flocculin essential for invasive growth and pseudohyphal development (Lo et al., Mol. Biol. Cell. 9:161-71 (1998), is controlled by a large regulatory region that is known to integrate multiple signal transduction pathways that are involved in environmental sensing and response, including pH, carbon, nitrogen and osmolarity (Gagiano et al., J. Bacteriol. 181:6497-6508 (1999); Rupp et al., EMBO J. 18:1257-69 (1999). Thus, genes that modulate expression of Flo11 may modulate expression of genes that influence secondary metabolite production. Several genes were identified using this method. As described in greater detail below and summarized in Table 2, several of these genes increased lovastatin production in A. terreus.

We generated cDNA libraries from RNA prepared from A. nidulans and P. chrysogenum fermentation biomass, and performed screens in S. cerevisiae to isolate clones that induced expression of the FLO11 promoter. In order to quantify the effects of the A. nidulans clones on FLO11 expression, cDNA expression plasmids were co-transformed with a FLO11ₚ-lacZ reporter construct into the yeast strain 10560-14C, and extracts were analyzed for β-galactosidase activity (results shown below in Table 3). Strains that increased FLO11-lacZ activity also showed increased invasiveness on agar plates, suggesting regulation of the chromosomal FLO11 locus (data not shown). The ORFs identified were subsequently referred to as rfe genes (Regulator of Flo 11 {Eleven}). Fourteen plasmids defining seven genes were isolated from the A. nidulans library, and representative clones were fully sequenced. Bioinformatic analysis identified motifs consistent with a regulatory role for RfeA, RfeB and RfeC. Sequences identical or related to rfeD, rfeE, rfeF and rfeG are present in the NCBI database as unannotated genomic clones from a number of fungal species. An identical screen performed using the P. chrysogenum cDNA expression library led to the isolation of 12 clones, three of which (rfeH (PC23), rfeI, rfeJ) contained open reading frames with homology to Zn-finger transcription factors.

TABLE 3

| Gene | # of | | Flo11-LacZ |
|---|---|---|---|
| ID | isolates | PFAM Hit (E-value) | induction |
| RfeA | 2 | PF00069: pkinase, Protein kinase domain | 1.7 |
| | | (9e−40) | |
| RfeB | 4 | PF00046: homeobox, Homeobox domain | 9.6 |
| | | (6e−06) | |
| RfeC | 3 | PF00096: Zinc finger, C2H2 type | 7.0 |
| | | (2.2e−05) | |
| RfeD | 1 | No significant homologies | 6.8 |
| RfeE | 1 | No significant homologies | 7 |
| RfeF | 2 | No significant homologies | 8.4 |
| RfeG | 1 | No significant homologies | 2.8 |
| Table 3: rfe genes isolated from A. nidulans based on the ability to regulate FLO11 expression. cDNA expression plasmids that increased expression from the FLO11ₚ-neo plasmid were isolated from S. cerevisiae and subject to first-pass sequencing. Representatives from each class were fully sequenced and nucleic acid and | | | |
| # deduced protein sequences for rfe genes were compared against protein and nucleic acid databases. PFAM homology and E-value are indicated where appropriate. The effects of rfe genes on FLO11 expression were quantified using a FLO11ₚ-lacZ reporter (see Materials and Methods.) | | | |

The rfe genes from both screens were subsequently introduced into A. terreus to assess their ability to modulate lovastatin production. In addition, to test whether increasing the potency of transcriptional regulators would affect metabolite production, putative transcription factors (RfeB, RfeC, RfeH (PC23), RfeI, and RfeJ) were also expressed as N-terminal fusions to the herpes simplex virus VP16 acidic domain, a region previously shown to increase transcriptional activation potential (Sadowski et al., Nature 335:563-64 (1988); Ma et al., Cell 55:443-46 (1988).

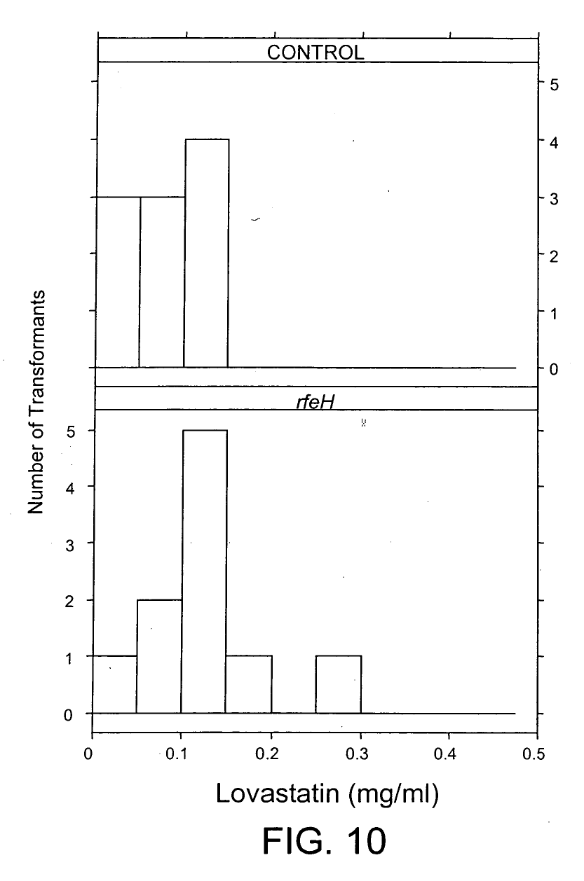

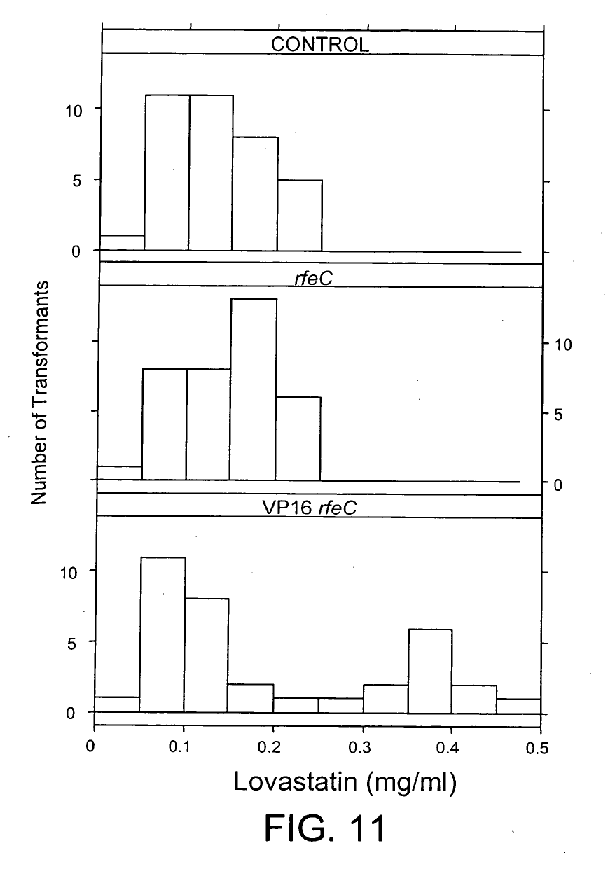

Unmodified RfeH (PC23) enhanced lovastatin production (FIG. 10), while VP16 activated RfeH (PC23) failed to modulate production of the metabolite (data not shown). Conversely, VP16 activated RfeC significantly enhanced production of lovastatin (p<0.01; FIG. 11), while the native gene had no observable effect. Activation-domain fusions to several other proteins either failed to stimulate metabolite production (e.g., RfeB) or eliminated the yield improvements that resulted from expression of the native protein (e.g., CreA, RfeH (PC23); data not shown).

### EXAMPLE 10

Genes that Modulate Lovastatin Production in A. terreus also Modulate Norsolorinic Acid Production in Aspergillus nidulans

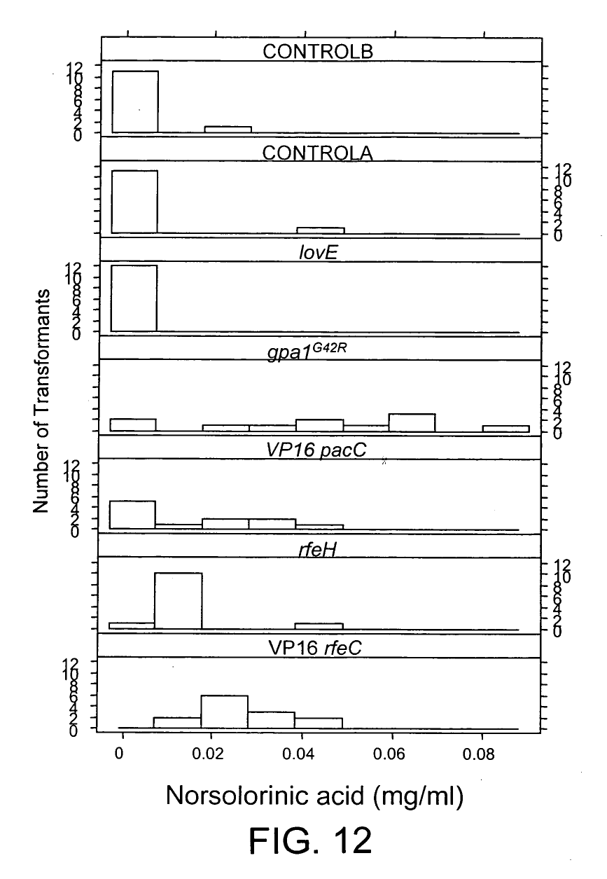

We examined several genes that activate lovastatin production to determine if they modulate production of norsolorinic acid, a colored intermediate in the sterigmatocystin biosynthetic pathway of A. nidulans. As shown in FIG. 12, transformants of ATJH3.40 containing VP16-rfeC, rfeH/Pc23, VP16-truncated pacC, and gpa1^{G42R} produced norsolorinic acid on a minimal medium that failed to induce measurable norsolorinic acid in control transformants. Interestingly, while two of these genes (VP16-rfeC and rfeH/Pc23) had previously been shown to enhance production of lovastatin, a third, gpa1^{G42R} acted to eliminate lovastatin production in A. terreus. The stimulatory effect of VP16-truncated PacC on norsolorinic acid was notable because truncated PacC (but not VP16-truncated PacC) acted to reduce lovastatin production in A. terreus. Transformations with lovE, areA, creA, fadAG^{42R}, ganB and gna3^{G44R} did not significantly alter norsolorinic acid yield.

### EXAMPLE 11

Modulation of (+)-Geodin Production

(+)-Geodin is derived from the octaketide anthraquinone emodin (Fujimoto et al., Chem Ber 108:1224-28 (1975), an intermediate in the biosynthesis of many natural products (Sankawa et al., Tetrahedron Lett 2125-28 (1973); Franck et al., Angew Chem 78:752-753 (1976); and Birch et al., J Chem Soc Perkin Trans 1:898-904 (1976)). We examined (+)-geodin production in A. terreus transformed with Gα proteins that increased (Gna3^{G44R}, GanB, and GanB^{G45R}) or decreased (FadA^{G42R}, Gna1^{G42R} and Gpa1^{Q204L}) lovastatin production, as well as transformants expressing CreA, RfeC and its chimeric VP16-RfeC analog, and LovE. In assessing geodin production transformants were compared to either transformants containing the appropriate null plasmid or to the wild-type A. terreus strain (MF22) (+)-Geodin production was analyzed by HPLC (UV detection) and LC/MS (high resolution electrospray quadropole time of flight mass spectrometry detection). Profiling by LC/MS identified a variety of (+)-geodin related compounds, with (+)-geodin itself being the most abundant secondary metabolite in broths from control strains. The identity of this compound was confirmed by ¹H nuclear magnetic resonance (NMR) spectroscopy. Mean molar concentrations of metabolites were determined by HPLC from a population of reference and engineered strains. Relative concentrations represent ratio of mean concentration in engineered strains relative to mean concentration in the appropriate reference strain. The results of this analysis are summarized below in Table 4.

TABLE 4
Modulation of (+)-geodin production

| | | GEODIN Relative |
|---|---|---|
| Engineered strain | Reference strain | concentration^{a} |
| MF22 + lovE(MF99) | MF22 | .42 |
| MF22 + VP16-rfeC | MF22 | .12 |
| MF22 + Pc23 | MF22 | NA |
| MF22 + creA | MF22 + control vector | 2.66 |
| MF22 + ganB | MF22 + control vector | 1.95 |
| MF22 + gna3G44R | MF22 + control vector | 1.62 |
| MF22 + ganBG45R | MF22 + control vector | 1.43 |
| MF22 + gpa1Q204L | MF22 | <0.02 |
| MF22 + gna1G42R | MF22 + control vector | <0.06 |
| MF22 + fadAG42R | MF22 | <0.02 |

### EXAMPLE 12

Modulation of the Production of Three Metabolites in A. terreus: N-Acety1va1y1-N-2-(1H-indol-3-yl)ethenyl]-NÉmethylphenylalaninamide (a modified tripeptide), Methyl 3,4,5-trimethoxy-2-[[2-[(3-pyridinylcarbonylamino]benzoyl]amino]benzoate (an alkaloid), and osoic acid 3-methyl ether, 1-methyl ester (a Polyketide).

N-Acetylvalyl-N-˜2-(1H-indol-3-yl)ethenyl]-N&-methylphenylalaninamide is an enzymatically modified tripeptide, likely to be non-ribosomal, that functions as an antinephritic agent (Japan. Pat., JP01149766 A2; Nakano, Yoshimasa; Sugawara, Michiharu (Otsuka Pharmaceutical Co., Ltd., Japan. 1989). Methyl 3,4,5-trimethoxy-2-[[2-[(3-pyridinylcarbonylamino]benzoyl]amino]benzoate is an alkaloid derived from anthranillic acid; related compounds have been shown to affect smooth muscle relaxation (Arai, K., et al., Chem. Pharm. Bull. v.29, p. 1005, 1981. Japan. Pat., JP56161362 A2; Kyoto Pharmaceutical Industries, Ltd., Japan. 1981). Osoic acid 3-methyl ether, 1-methyl ester is a Well studied polyketide of A. terreus that has been shown to function as an endothelin binding inhibitor (Ohashi, H., et al., Journal of Antibiotics v.45(10), p. 1684. 1992). We examined the production of these metabolites in A. terreus transformed with Gα proteins that increased (Gna3^{G44R}, GanB, and GanB^{G45R}) or decreased (FadA^{G42R}, Gna1^{G42R} and Gpa1^{Q204L}) lovastatin production, as well as transformants expressing CreA, RfeC and its chimeric VP16-RfeC analog, and LovE. These metabolites were identified in the extracted culture broths of these engineered strains using LCMS (described in example E4), by comparison of chromatographic peaks observed in high-resolution extracted mass chromatograms (Micromass Metabolynx software) with the masses of known A. terreus secondary metabolites (Chapman and Hall Dictionary of Natural Products, version 10:2, February 2002, CRC Publ.). Chromatographic resolution of the compounds was achieved with a 16 minute isocratic elution in 60% acetonitrile (0.1% formic acid): 40% aqueous 0.1% formic acid at a flow rate of 0.2 mL/min over a reverse phase HPLC column (Waters Xterra, 5 μm, 2.1×250 mm) Each metabolite was detected reproducibly, with high mass precision (<10 ppm). In assessing metabolite production, transformants were compared to either transformants containing an analogous control plasmid or to the wild-type A. terreus strain, and no absolute determination of metabolite concentration was attempted with these metabolites. The effects of various genes were found to be highly specific. For example, creA caused an increase in the production of the tripeptide, but had no effect on osoic acid or alkaloid production. In addition, the modulation of lovastatin and geodin described in earlier examples was re-observed using this method of metabolite detection and tracking. See Table 2 for complete results.

### EXAMPLE 13

Overexpression of gpa3 or Lys14 Improves Lovastatin Production in A. terreus

The effect of A. nidulans gpa3 overexpression and S. cerevisiae lys14 overexpression on lovastain production in A. terreus was examined by transforming A. terreus strain MF22 (ATCC 20542) with a plamid bearing gpa3 (MB3250) or a plamid bearing LYS14 (MB1669). The results of this analysis are summarized in Table 2. Both gpa3 and lys14 overexpression increased lovastatin production.

For transformation of A. terreus protoplasts were generated from spores that were germinated in rich media. Spores were allowed to germinate for about 20 hrs or until germ tubes were between 5 and 10 spore lengths. The germlings were centrifuged and washed twice with sterile distilled water and once with 1M magnesium sulfate. Germlings were then resuspended in 1M magnesium sulfate containing approximately 2 mg/ml of Novozyme. Tubes were then incubated at 30° C. shaking at 80 rpm for about 2 hours or until most of the hyphae were digested and protoplasts were abundant. Protoplasts were then filtered through one layer of Miracloth. At least one volume of STC (0.8M Sorbitol, 25 mM Tris-HCl pH 7.5, 25 mM CaCl₂) was added and protoplasts were centrifuged. Protoplasts were washed twice with STC. Protoplasts then were resuspended in 1 ml STC and counted in a hemocytometer. A final concentration of approximately 5×10⁷ protoplasts/ml was frozen in a 9:1:0.1 solution of STC, SPTC (0.8M Sorbitol, 40% PEG 4000, 25 mM Tris-HCl pH 8, 50 mM CaCl₂) and DMSO in a Nalgene Cryo cooler at −80° C. (cools −1° C./min). Next, 1-5 μg of DNA comprising the designated plasmid was placed in a 50 ml Falcon tube. 100 μl of previously frozen protoplasts were added to the DNA, gently mixed, and then incubated on ice for 30 minutes. 15 μl of SPTC was added, followed by mixing (by tapping) and incubation at RT for 15 minutes. 500 μl SPTC was added and mixed well by tapping and rolling, then incubated at RT for 15 minutes. 25 mls of regeneration minimal medium was added, mixed well and poured on plates containing 25 mls of regeneration minimal medium with 2× the concentration of selection drug.

Transformation plates containing phleomycin, a broad-spectrum glycopeptide antibiotic, were incubated at 26° C. for 5-6 days or until colonies started to appear. Regeneration minimal medium contains trace elements, salts, 25 mM sodium nitrate, 0.8M sucrose, and 1% agarose at pH 6.5. Transformants were picked onto new plates with a toothpick (if fungus was sporulating) or with sterile forceps (if fungus did not sporulate). Purification plates contained minimal medium (same as regeneration minimal medium but containing 2% instead of 0.8M sucrose) and 1× drug concentration. Picked transformants were incubated at 26° C. for 5-6 days.

Transformants were grown in production media to assess secondary metabolite production. Briefly, for A. terreus and lovastatin production, spores were used as the inoculum. Spores were obtained from the purification plate by using a wooden inoculation stick. The medium was RPM containing corn steep liquor, sodium nitrate, potassium phosphate, magnesium sulfate, sodium chloride, P2000 (Dow Chemical), trace elements and lactose or glucose as carbon source. The medium was pH 6.5. Flasks were incubated at 26° C. with shaking at 225 rpm. For static 96-well cultures, the same medium was used and the spores were obtained from the purification plate with a wooden toothpick. 96-well plates were incubated, without shaking, at 26° C.

PCR analysis of transformants demonstrates that greater than fifty percent of the transformants contain the transgene. Variability in levels of transgene expression can presumably be influenced by integration site and copy number

Sampling for measurement of lovastatin production was done after 5 days for lovastatin. For shake flask experiments 1-1.5 mls of supernatant was placed into 96-well plates, which were centrifuged and supernatants transferred to new 96-well plates. Samples were frozen at −80° C. for storage and for later assays. Cultures that were grown standing in a 96-well plate were centrifuged and the supernatant was transferred to a new 96 well plate. Samples were frozen at −80° C.

Lovastatin concentration was determined by high pressure liquid chromatography (HPLC). Briefly, 100 μL of broth sample was removed and diluted 1:10 into 70% H₂O-30% acetonitrile (900 μl). This mixture was centrifuged to pellet debris at 13000 rpm for 5 minutes. 900 μl of this diluted broth was transferred to a vial and the sample was analyzed by HPLC. 10 μl were injected into a Waters HPLC system (996 photo-diode array detector, 600 E pump controller and 717 autosampler) equipped with a YMC-Pack ODS column (Aq-302-3, 150×4.6 mm ID, S-3 μM pore size) and eluted with isocratic 40% aqueous acetic acid (0.7%)-60% acetonitrile for 8 minutes. Lovastatin was detected at 238 nm and was shown to have a retention time of 6.5 minutes. Lovastatin in samples was quantified using a calibration curve created from pure lovastatin samples.

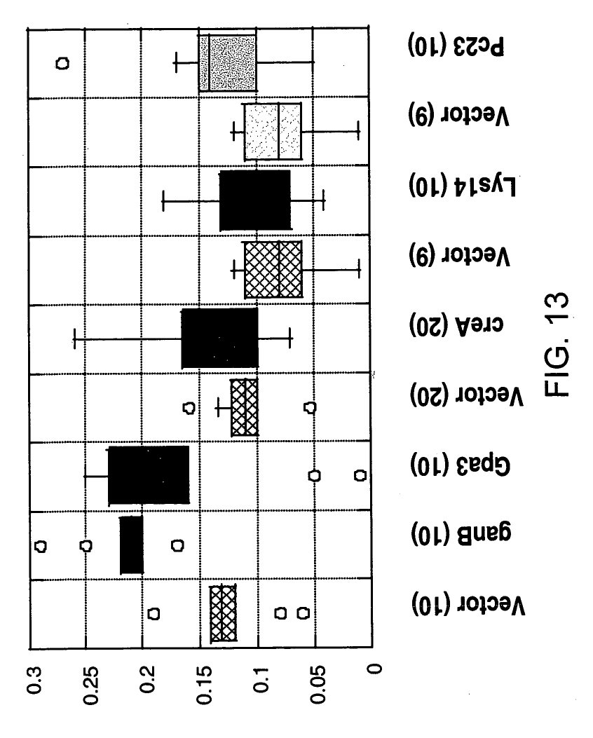

The results of this study are shown in FIG. 13 which is a graphic depiction of lovastatin culture concentration, as measured by HPLC analysis, from broths of A. terreus cultures expressing the regulators. The number of different transformants tested for each plasmid is listed in parentheses next to the label. Results are shown in standard box plot format. The horizontal line in each individual box represents the median. The corresponding vector control is shown in a hatched same colored box.

### EXAMPLE 14

Modulation of Penicillin Production in P. chrysogenum

The effect of overexpression of A. nidulans creA, A. terreus LovE, and A. terreus orf13/lovU, a protein related to lovE on penicillin production by P. chrysogenum was investigated as follows. Plasmids MB1325 (vector control), MB1310 (creA), MB1316 (lovE), and MB1317 (orf13) were transformed into P. chrysogenum strain MF1 (ATCC 9480). The results of this analysis are summarized in Table 2. Overexpression of A. nidulans creA, A. terreus LovE, A. terreus orf13/lovU increased penicillin production in P. chrysogenum

P. chrysogenum was transformed as described immediately above for A. terreus, except transformants were selected on 30 μg/mL phleomycin. Samples for testing penicillin production were obtained by using a plug containing spores and mycelia is used as the culture inoculum. The medium used is the published P2 production medium (Lein (1986), in Overproduction of Microbial Metabolites, Vanek and Hostalek (eds.), Butterworth Heinemann, pp. 105-139) that contains 30% lactose, 5× pharmamedia cotton seed flour, ammonium sulfate, calcium carbonate, potassium phosphate, potassium sulfate, and phenoxyacetic acid, at pH 7. Flasks were incubated at 26° C. with shaking at 225 rpm. Sampling was done after 6 days of growth. 1-1.5 mls of supernatant were placed into 96-well plates. Plates were centrifuged and supernatants transferred to a new 96-well plate for the penicillin assay.

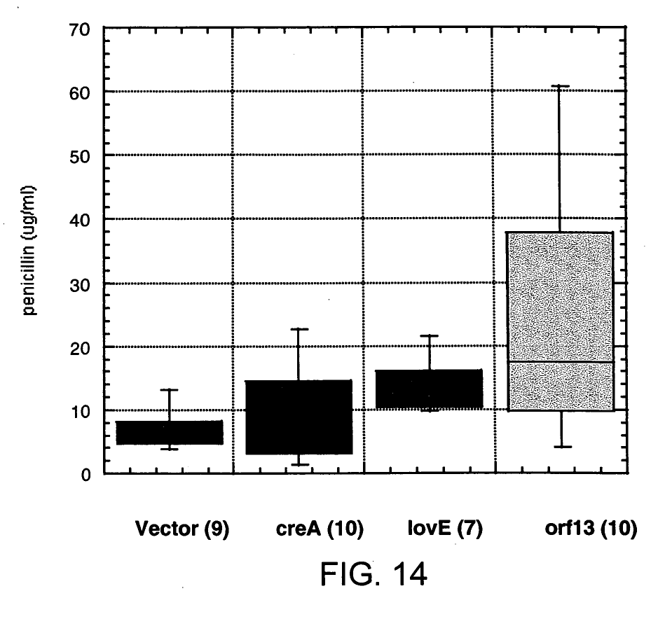

Standard samples for the penicillin assay contained 0, 25, 50, 100, 200, 300, 400, and 500 μg/mL phenoxymethylpenicillin (sodium salt) dissolved in 10 mM potassium phosphate (pH 7.0). Fermentation broth from test samples was clarified by centrifugation for 10 minutes at 4000 g, and 40 μL of clarified fermentation broth and penicillin standard solutions were pipetted into individual wells of a 96-well UV collection plate. Next, 200 μL of imidazole reagent was pipetted into a 96-well filter plate (0.45 micron). The derivatization reaction of penicillin was initiated by vacuum filtration of imidazole reagent into a collection plate containing the aliquoted samples and standards. The collection plate was placed into a 96-well plate reader at 45 degrees while absorbance at 325 nm was monitored over 20 minutes. A Molecular Dynamics (Sunnyvale, Calif.) 96-well UV/is plate reader was used for all spectrophotometric detection. A 1.2 M aqueous imidazole solution containing mercuric chloride at a concentration of 1 mM, pH 6.8 was prepared as follows: 8.25 g of imidazole was dissolved in 60 mL of water, 10 mL of 5 M HCl was added, and then 10 mL of a solution of mercuric chloride (0.27 g dissolved in 100 mL of water) was added. The pH was adjusted to 6.80+/−0.05 with 5 M HCl and the volume was brought to 100 mL with water (see, e.g., Bundgaard and Ilver (1972), Journal of Pharm. Pharmac: 24: 790-794). The results of this study are shown in FIG. 14 a graphic depiction of penicillin culture concentration, as measured by UV/Spec analysis, from broths of P. chrysogenum cultures expressing the regulators. The number of individual transformants tested for each plasmid is listed in parentheses next to the label. Results are shown in standard box plot format. The horizontal line in each individual box represents the median.

The results demonstrate that the three heterologous regulators increase penicillin production in P. chrysogenum. Genes for two of these regulators, lovE and orf13 are present in the biosynthetic cluster for another secondary metabolite, lovastatin.

### EXAMPLE 15

Additional Regulators that Increase Lovastatin Production

The effect of expression of Ustilago maydis ste7, S. cerevisiae vps34, N. crassa nc1, and S. cerevisiae pde2 on lovastatin production in A. terreus. The preparation of expression contructs, transformation, culturing and lovastatin measurement methods were similar to those described above.

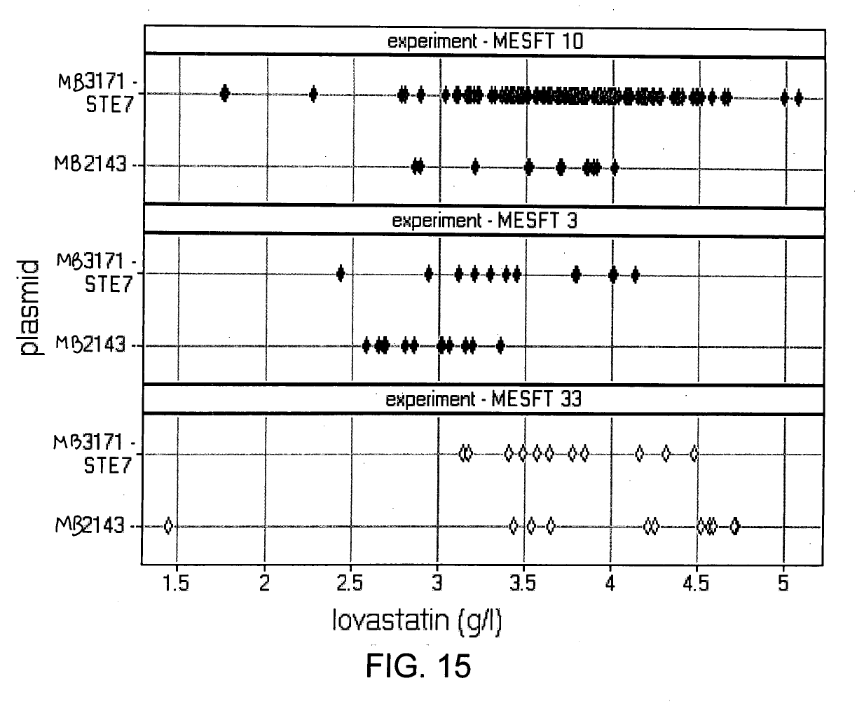

FIG. 15 depicts the results of three different studies (MESFT 10, MESFT 3, and MESFT 33) in which production of lovastatin was measured in numerous different transformants (each represented by a diamond) harboring either a vector expressing STE7 (MB3171-STE7) or a control vector (MB2143). These studies demonstrate that Ustilago maydis ste7 can increase lovastatin production in A. terreus.

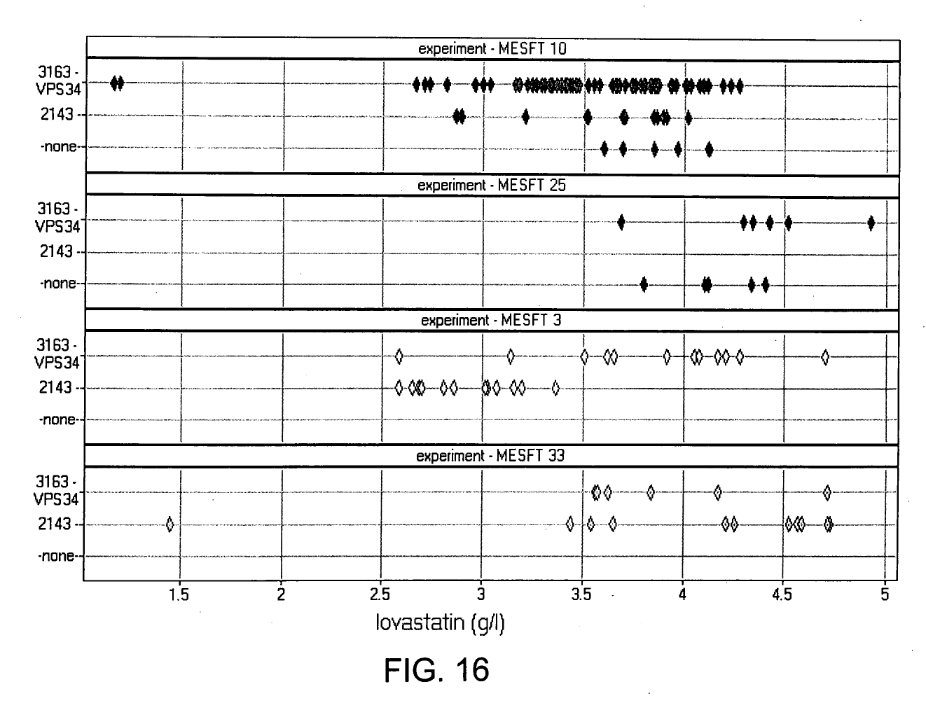

FIG. 16 depicts the results of four different studies (MESFT 10, MESFT 25, MESFT3 and MESFT 33) in which production of lovastatin was measured in numerous different transformants (each represented by a diamond) harboring either a vector expressing VPS34 (MB3163-VPS34) or a control vector (MB2143) or no vector (none). These studies demonstrate that S. cerevisiae vps34 can increase lovastatin production in A. terreus.

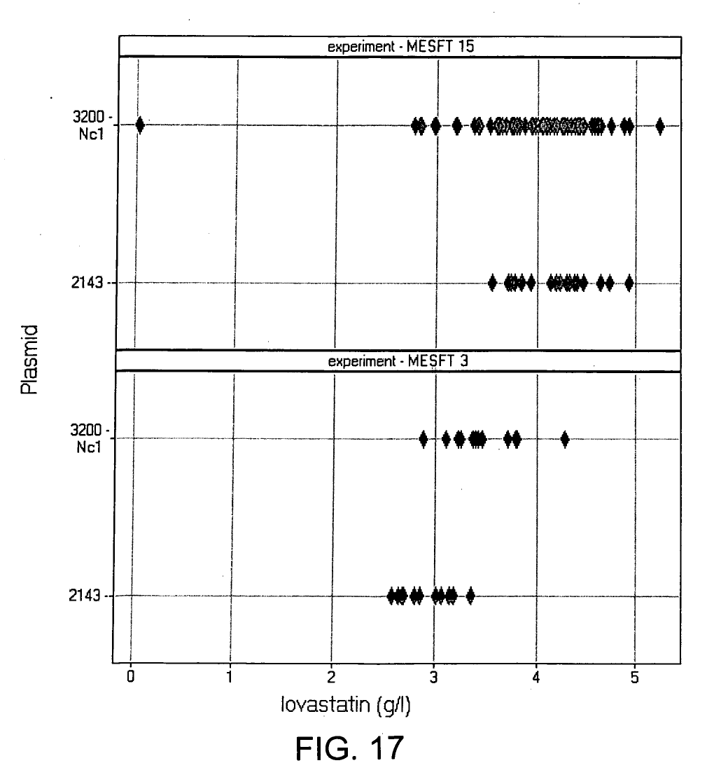

FIG. 17 depicts the results of two different studies (MESFT 15 and MESFT 3) in which production of lovastatin was measured in numerous different transformants (each represented by a diamond) harboring either a vector expressing Nc1 (MB3200-Nc1) or a control vector (MB2143). These studies demonstrate that N. crassa nc1 can increase lovastatin production in A. terreus.

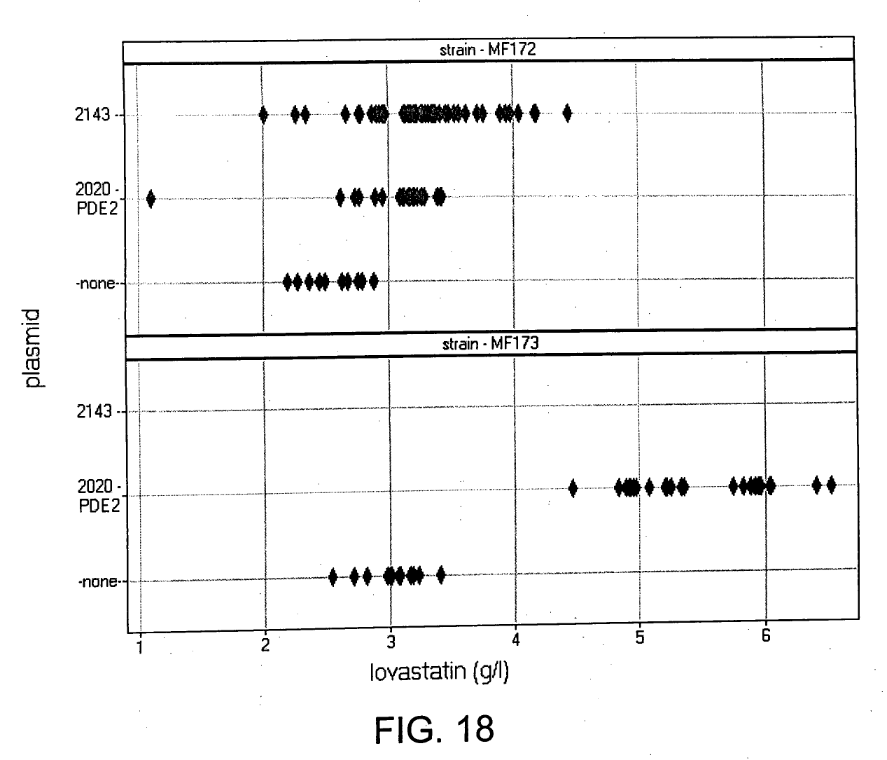

FIG. 18 depicts the results of two studies in two different strains (MF172 and MF 173) in which production of lovastatin was measured in numerous different transformants (each represented by a diamond) harboring either a vector expressing PDE2 (MB2020-PDE2) or a control vector (MB2143) or no vector (none). These studies demonstrate that S. cerevisiae pde2 can increase lovastatin production in A. terreus.

A number of embodiments of the invention have been described. Nevertheless, it will be understood that various modifications may be made without departing from the spirit and scope of the invention. Accordingly, other embodiments are within the scope of the following claims.

What is claimed is:

1. A method for modulating production of a secondary metabolite by a fungus, the method comprising increasing the expression or activity of a protein selected from the group consisting of CreA, AreA, GanB, Gna3, FadA, Gna1, Gpa1, RfeH, RfeC, PacC, Lys14, lovU, Ste7, Pde2, Nc1, Vps34, lovE or fungal homologs thereof, provided however, that when the secondary metabolite is sterigmatocystin, then the protein is not FadA; when the secondary metabolite is penicillin and the fungus is Aspergillus nidulans, then the increase in activity is not through mutations that result in expression of truncated forms of PacC or constitutively active forms of FadA; and that when the secondary metabolite is lovastatin and the fungus is Aspergillus terreus, then the increase in expression is not in lovE.

2. The method according to claim 1, wherein expression of the protein is increased.

3. The method according to claim 1, wherein activity of the protein is increased.

4. The method according to claim 1, wherein the protein is encoded by a gene having a dominant negative mutation.

5. The method according to claim 1, wherein the protein is encoded by a gene having a dominant positive mutation.

6. The method of claim 1 wherein the secondary metabolite is a polyketide.

7. The method of claim 6 wherein the polyketide is a statin.

8. The method of claim 6 wherein the polyketide is geodin.

9. The method of claim 6 wherein the polyketide is lovastatin.

10. The method of claim 6 wherein the polyketide is norsolorinic acid.

11. The method of claim 6 wherein the polyketide is osoic acid.

12. The method of claim 1 wherein production of the secondary metabolite is increased.

13. The method of claim 1 wherein expression of the secondary metabolite is decreased.

14. The method of claim 1, further comprising the step of purifying the secondary metabolite from a culture of the fungus.

15. A method for producing a secondary metabolite comprising:
(a) providing a fungal strain harboring a recombinant nucleic acid molecule encoding a protein selected from the group consisting of: CreA, AreA, GanB, Gna3, FadA, Gna1, Gpa1, RfeH, RfeC, PacC, Lys14, LovU, Ste7, Pde2, Nc1, Vps34;
(b) culturing the fungal strain under conditions for producing the secondary metabolite; and
(c) purifying the secondary metabolite from the culture.

16. A method for producing a fungal strain having altered production of a secondary metabolite, the method comprising transforming a fungal strain with a nucleic acid molecule encoding a protein selected from the group consisting of CreA, AreA, GanB, Gna3, FadA, Gna1, Gpa1, RfeH, RfeC, PacC, Lys14, LovE, LovU, Ste7, Pde2, Nc1, Vps34 and fungal homologs thereof.

17. The method of claim 15 or 16 wherein the fungus is A. terreus.

18. The method of claim 17 wherein the secondary metabolite is a polyketide.

19. A method for improving production of a secondary metabolite by a fungus by increasing the yield of the secondary metabolite in the fungus, the method comprising modulating the expression of a gene involved in regulation of secondary metabolite production in a manner that improves the yield of the secondary metabolite, provided however, that when the secondary metabolite is isopenicillin N, then the modulation is not mediated by transcription factor CPCR1; when the secondary metabolite is sterigmatocystin, then the modulation is not through AflR, FadA, or FluG; when the secondary metabolite is aflatoxin, then the modulation is not through AflR; when the secondary metabolite is penicillin and the fungus is Aspergillus nidulans, then the modulation is not through mutations that result in expression of truncated forms of PacC or constitutively active forms of FadA; and when the gene involved in regulation of secondary metabolite production is from Saccharomyces cerevisiae, then the modulation is not through decreased activity or expression of Hog1, Bem2, Rim15, Sfl1, Ira1, Ssd1, Srb11, Swi4, Tpk3 or though increased activity or expression of Afl1, Dhh1, Inv7, Inv8, Ste21, Pet9, Mep2, Inyl, Inv5, Inv6, Inv9, Inv10, Inv11, Inv12, Inv13, Inv14, Inv15, Cdc25, Mcm1, Mga1, Phd2, Pho23, Ptc1, Rim1, Stp22, Tpk2 or Ypr1.

20. The method according to claim 19, wherein the modulation is overexpression of the gene.

21. The method according to claim 19, wherein the modulation is conditional expression of the gene.

22. The method according to claim 19, wherein the modulation is expression of a dominant mutation of the gene.

23. The method according to claim 22, wherein the dominant mutation is a dominant negative mutation.

24. The method according to claim 22, wherein the dominant mutation is a dominant positive mutation.

25. The method according to claim 22 wherein the dominant mutation is a dominant neomorphic mutation.

26. The method according to claim 19, wherein the modulation is mediated by a peptide modulator of gene expression.

27. The method according to claim 26, wherein the peptide modulator is an activator of gene expression.

28. The method according to claim 26, wherein the peptide modulator is an inhibitor of gene expression.

29. The method according to claim 19, wherein the modulation is mediated by a small molecule modulator of gene expression.

30. A method for improving production of a secondary metabolite by a fungus by increasing productivity of the secondary metabolite in the fungus, the method comprising modulating the expression of a gene involved in regulation of secondary metabolite production in a manner that improves the productivity of the secondary metabolite, provided however, that when the secondary metabolite is isopenicillin N, then the modulation is not mediated by transcription factor CPCR1; when the secondary metabolite is sterigmatocystin, then the modulation is not through AflR, FadA, or FluG; when the secondary metabolite is aflatoxin, then the modulation is not through AflR; when the secondary metabolite is penicillin and the fungus is Aspergillus nidulans, then the modulation is not through mutations that result in expression of truncated forms of PacC or constitutively active forms of FadA; and when the gene involved in regulation of secondary metabolite production is from Saccharomyces cerevisiae, then the modulation is not through decreased activity or expression of Hog1, Bem2, Rim15, Sfl1, Ira1, Ssd1, Srb11, Swi4, Tpk3 or though increased activity or expression of Afl1, Dhh1, Inv7, Inv8, Ste21, Pet9, Mep2, Inyl, Inv5, Inv6, Inv9, Inv10, Inv11, Inv12, Inv13, Inv14, Inv15, Cdc25, Mcm1, Mga1, Phd2, Pho23, Ptc1, Rim1, Stp22, Tpk2 or Ypr1.

31. A method for improving production of a secondary metabolite in a fungus by increasing efflux or excretion of the secondary metabolite, the method comprising modulating the expression of a gene involved in regulation of secondary metabolite production in a manner that increases efflux or excretion the secondary metabolite.

32. A method for improving production of a secondary metabolite in a fungus by decreasing production of side products or competing secondary metabolites, the method comprising modulating the expression of a gene involved in regulation of secondary metabolite production in a manner that decreases production of side products or competing secondary metabolites.

33. A method for improving production of a secondary metabolite in a fungus by altering the characteristics of the fungus in a manner that is beneficial to the production of the secondary metabolite, the method comprising modulating the expression of a gene involved in regulation of secondary metabolite production in a manner that alters the characteristics of the fungus.

34. A method for improving production of a secondary metabolite in a fungus by causing conditional lysis of the fungus, the method comprising modulating the expression of a gene involved in regulation of secondary metabolite production in a manner that causes conditional lysis.

35. A method for improving production of a secondary metabolite in a fungus by increasing the resistance of the fungus to the deleterious effects of exposure to a secondary metabolite, the method comprising modulating the expression of a gene involved in regulation of secondary metabolite production in a manner that increases resistance to the deleterious effects of exposure to a secondary metabolite.
